(19) United States
(12) Patent Application Publication
(10) Pub. No.: US 2022/0236281 A1
(43) Pub. Date: Jul. 28, 2022

(54) METHOD, STRUCTURES AND SYSTEM FOR NUCLEIC ACID SEQUENCE TOPOLOGY ASSEMBLY FOR MULTIPLEXED PROFILING OF PROTEINS

(71) Applicants: NATIONAL UNIVERSITY OF SINGAPORE, Singapore (SG); NATIONAL UNIVERSITY HOSPITAL (SINGAPORE) PTE. LTD., Singapore (SG)

(72) Inventors: Huilin SHAO, Singapore (SG); Noah Riandiputra SUNDAH, Singapore (SG); Ching Wan CHAN, Singapore (SG); Tze Ping LOH, Singapore (SG)

(73) Assignees: NATIONAL UNIVERSITY OF SINGAPORE, Singapore (SG); NATIONAL UNIVERSITY HOSPITAL (SINGAPORE) PTE. LTD., Singapore (SG)

(21) Appl. No.: 17/615,115

(22) PCT Filed: May 19, 2020
(86) PCT No.: PCT/SG2020/050298

§ 371 Date: Nov. 30, 2021

(30) Foreign Application Priority Data
May 30, 2019 (SG) 10201904897U

(51) Int. Cl. G01N 33/68; G01N 33/543; G01N 33/53

(52) U.S. Cl. CPC G01N 33/6842; G01N 33/5308; G01N 33/54346

(57) ABSTRACT

Methods and systems including a set of interacting nucleic acid structures for use in detecting and/or identifying a target comprising: a nucleic acid sequence capable of being conjugated to a moiety directed to the target; and a nucleic acid nanostructure comprising a segment sequence complementary to a portion of the nucleic acid sequence capable of being conjugated to a moiety directed to the target. The moiety directed to the target may be an antibody, and the nucleic acid nanostructure may be a tetrahedron.

### CROSS REFERENCE TO RELATED APPLICATIONS

This application claims the priority to Singapore patent application No. 10201904897U, filed 30 May 2019, the contents of which are incorporated herein by reference.

### FIELD

The invention relates to methods, structures and systems for detecting and/or identifying proteins.

### Background

The following discussion of the background to the invention is intended to facilitate understanding of the present invention. However, it should be appreciated that the discussion is not an acknowledgment or admission that any of the material referred to was published, known or a part of the common general knowledge in any jurisdiction as at the priority date of the application.

Comprehensive analysis of protein expression and distribution holds promises for discovery of biomarker panels, early disease detection, and rational selection of personalized treatment (Borrebaeck, C. A. Nat Rev Cancer 17, 199-204 (2017).). However, unlike genomic analysis, where various analytical platforms (e.g., next generation sequencing and quantitative polymerase chain reaction, qPCR) are well established for massively parallel DNA measurements (Klein, A. M. et al. Cell 161, 1187-1201 (2015)), high-throughput protein profiling remains a challenging task and has limited sensitivity, especially for detecting rare protein targets and scant cells (Kingsmore, S. F. Nat Rev Drug Discov 5, 310-320 (2006)). In contrast to advances in massively parallel nucleic acid sequencing, high-throughput protein analysis remains limited.

DNA barcoding offers an attractive approach to bridge the gap between genomic and proteomic analyses (Nong, R. Y., et al. Expert Rev Proteomics 9, 21-32 (2012)). In this approach, protein-targeting antibodies can be attached with DNA barcodes; this information transfer, from proteomics to genomics, enables signal amplification and detection, all through established DNA analytical platforms. Despite such potential, current DNA barcoding technologies have limitations, especially for high-throughput analysis of subcellular protein distribution. First, to generate compatible DNA barcodes of sufficient sequence length and variability, long linear DNA strands are commonly used for direct antibody barcoding. These complexes have poor intracellular stability and functionality (Boutorine, A. S., et al. Molecules 18, 15357-15397 (2013))16, thereby limiting their utility for protein measurements. The current antibody-DNA approaches are designed to ensure compatibility with direct PCR amplification (Fredriksson, S. et al. Nat Methods 4, 327-329 (2007)). These antibody-DNA complexes have reduced performance. In attempts to preserve antibody function, current assays rely on stringent antibody selection and/or dedicated conjugation processes, such as site-specific antibody modifications (Kazane, S. A. et al. Proc Natl Acad Sci USA 109, 3731-3736 (2012)), ribosome display (Gu, L. et al. Nature 515, 554-557 (2014))19 and advanced nucleic acid chemistries (Agasti, S. S., et al. J Am Chem Soc 134, 18499-18502 (2012)), have been developed. Such approaches are complex, difficult to scale or multiplex, and cannot be easily generalized (Wu, A. M. & Senter, P. D. Nat Biotechnol 23, 1137-1146 (2005)) to existing antibody repertoire. It is not possible to use existing technology with just any off the shelf antibody.

For assay performance, existing antibody-DNA assays can be performed in the solution phase (Fredriksson, S. et al. Nat Biotechnol 20, 473-477 (2002)) or localized by microscopy (Goltsev, Y. et al. Cell 174, 968-981.e15 (2018)). While the solution-phase approaches enable multiplexed protein quantitation, they generally do not provide any information on protein subcellular localization. The microscopy assays can be adapted for subcellular distribution analysis; however, they have a limited throughput and multiplexing capability (e.g., spectral overlaps of fluorochrome labels).

Comprehensive analysis of subcellular protein expression and distribution holds promises for discovery of biomarker panels, early disease detection, and rational selection of personalized treatment (Landegren, U., et al. N Biotechnol 45, 14-18 (2018)). In particular, current proteomic approaches face limitations in performing multiplexed protein analysis at a subcellular resolution (Hughes, A. J. et al. Nat Methods 11, 749-755 (2014)). Conventional assays, such as flow cytometry and cell imaging, rely primarily on optical detection of targeted antibody binding. While these methods can be adapted for measuring subcellular protein expression and/or distribution in whole cells, they are limited by the number of spectrally non-overlapping fluorochrome labels and thus have limited multiplexing capability (Adan, A., et al. Crit Rev Biotechnol 37, 163-176 (2017)). Mass spectrometry can help to circumvent these optical challenges for parallel analysis; however, to map protein distribution within cells, the technology requires extensive sample processing (e.g., isolation of different subcellular compartments before peptide digestion (Foster, L. J. et al. Cell 125, 187-199 (2006)).

DNA stores dense genetic information and has the most predictable and programmable interactions of any natural or synthetic molecule to fold into precise structures. Despite successes in engineering DNA nanostructures of diverse shapes and sizes, their applications in multiplexed protein detection remain limited, and when possible, focused primarily on using the nanomaterials as a structural scaffold/motif for spectroscopy or microscopy measurements (Pei, H., et al. Acc Chem Res 47, 550-559 (2014).

Thus, there exists a need to develop methods and systems which ameliorates at least one of the disadvantages outlined above.

### Summary

An object of the invention is to ameliorate some of the above-mentioned difficulties preferably methods and systems for detecting and/or identifying proteins using any antibody.

One aspect of the invention relates to a method for detecting and/or identifying a target protein in a sample comprising: (a) forming a modified moiety by conjugating a nucleic acid sequence to an moiety directed to the target; (b) forming a nucleic acid nanostructure comprising a segment sequence complementary to a portion of the nucleic acid sequence of the modified moiety; (c) incubating the sample with the modified moiety to form a complex between the modified moiety and the target; (d) removing modified moieties that do not form a complex with the target; (e) allowing the complementary segment sequence of the nanostructure to hybridize to the portion of the nucleic acid of the modified moiety to which it is complementary; (f) forming a nucleic acid barcode comprising the complementary segment sequence of the nanostructure; and (g) detecting the nucleic acid barcode, whereby the detection of the nucleic acid barcode indicates that the target protein is present in the sample.

Another aspect of the invention relates to a set of interacting nucleic acid structures for use in detecting and/or identifying a target comprising: (a) a nucleic acid sequence capable of being conjugated to a moiety directed to the target; and (b) a nucleic acid nanostructure comprising a segment sequence complementary to a portion of the nucleic acid sequence capable of being conjugated to a moiety directed to the target.

Another aspect of the invention relates to a system for detection and/or identification of a target in a sample comprising: (a) a mixing chamber for mixing the sample with a modified moiety comprising a nucleic acid sequence conjugating to a moiety directed to the target; (b) a filter for capturing a complex between the modified moiety and the target; (c) a reservoir for incubating the moiety complex with a nucleic acid nanostructure comprising a segment sequence complementary to a portion of the nucleic acid sequence of the modified moiety and optionally at least two location identifiers; and (d) a detection chamber for detecting a nucleic acid barcode comprising the nucleic acid segment sequence complementary to the portion of the nucleic acid of the modified antibody.

Another aspect of the invention relates to a method of diagnosing a disease comprising: (a) forming a modified antibody by conjugating a nucleic acid sequence to an antibody directed to a target protein associated with the disease; (b) forming a nucleic acid nanostructure comprising a segment sequence complementary to a portion of the nucleic acid sequence of the modified antibody; (c) forming at least two location identifiers, wherein each location identifier comprises a nucleic acid sequence comprising a segment sequence complementary to a second portion of the nucleic acid sequence of the modified antibody and a unique identifier (d) incubating a sample with the modified antibody to form a complex between the modified antibody and the target protein, (e) removing modified antibodies that do not form a complex with the target protein (f) incubating the complex with the nucleic acid nanostructure, and at least one location identifier to form a super complex between the modified antibody, the target protein and the location identifier; (g) ligating the nucleic acid of the super-complex between the complementary segment sequence of the nucleic acid nanostructure and the complementary segment sequence of the location identifier; (h) forming a nucleic acid barcode comprising the ligated sequence complementary to the segment sequence of the nanoparticle and the sequence complementary to the segment sequence of the first, second or third location identifiers; and (i) detecting and analyzing the nucleic acid barcodes to determine the amount and/or subcellular distribution of target proteins, whereby the amount and/or subcellular distribution of target protein indicates the disease.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention include,

FIG. 1. DNA sequence-topology assembly for multiplexed profiling (STAMP) (a) Schematic representation of the STAMP assay and (b) Photograph of the STAMP microfluidic device. Inset shows human breast cancer cells (SKBR3) trapped on the porous membrane and stained with antibodies (anti-HER2,) and nuclear dye (Hoechst 33342). Scale bar in the inset: 50 μm.

FIG. 2. Assembly of the DNA nanostructure probe. (a) Synthesis of the DNA tetrahedral nanostructure. (b) Native polyacrylamide gel electrophoresis (PAGE) analysis of the DNA assembly. (c) Characterization of the DNA tetrahedron probes.

FIG. 3. Design of the microfluidic device. (a) Schematic representation of the microfluidic device. (b) Exploded view of the device.

FIG. 4. Operation schematic of the microfluidic STAMP platform.

FIG. 5. Highly sensitive protein detection with STAMP (a) Antibody-antigen binding kinetics; (b) Comparison of antibody performance on cellular targeting; (c) Comparison of linear and nanostructure DNA probes; (d) Evaluation of STAMP performance; and (e) Single-cell detection sensitivity of STAMP.

FIG. 6. Binding kinetics of antibody-DNA conjugates for (a) HER2 and (b) EGFR. All modified antibodies were conjugated with a comparable number of DNA strands.

FIG. 7. Biophysical characterization of antibody-DNA conjugation. (a) Comparison of kobs values of different antibody-DNA conjugates. Changes in (b) hydrodynamic diameter and (c) zeta potential of the antibody-DNA conjugates, as determined by dynamic light scattering analysis. All data are presented as mean t s.d. (*q<0.05, ***q<0.0005, not significant (n.s.), Kruskal-Wallis test).

FIG. 8. Binding kinetics of antibodies conjugated with varying number of DNA strands. Real-time binding kinetics of anti-HER2 antibody and antibody-DNA conjugates with varying number of (a) short DNA strands and (b) long DNA strands. All measurements were normalized against that of equivalently modified IgG isotype control antibodies. MFI, mean fluorescence intensity.

FIG. 9. Cellular targeting with antibody-DNA conjugates. Antibody-DNA conjugates (anti-HER2) with varying number of FAM-labeled short and long DNA strands were used to target different cell lines of known HER2 expression levels: (a) MDA-MB-231 (low HER2 expression), (b) SKBR3 (medium HER2 expression), and (c) SKOV3 (high HER2 expression).

FIG. 10. Flow cytometry and qPCR optimization of antibody-DNA conjugates. SKOV3 cells were targeted with anti-HER2 antibodies conjugated with varying numbers of short DNA strands per antibody. (a) Fluorescence measurements and (b) STAMP measurements were performed. STAMP analysis was performed via qPCR. All measurements were normalized against that of equivalently modified IgG isotype control antibodies. All measurements were performed in triplicates, and the data are presented as mean t s.d.

FIG. 11. Nanostructure-assisted ligation. DNA probe ligation was performed via (a) enzymatic ligation through T4 DNA ligase, and (b) chemical ligation through copper(I)-catalyzed alkyne-azide cycloaddition (CuAAC, or click chemistry). Note that the negative controls generated no detectable signals, in the absence of complementary target. All measurements were performed in triplicates, and the data are presented as mean±s.d. (*P<0.05, ***P<0.0005, Student's t-test). N.D.=not detected.

FIG. 12. Evaluation of STAMP performance. (a) immunolabeling with direct conjugate (Ab-long DNA), and the STAMP assay in PBS. (b) DNA tetrahedron (nanostructure) and single-stranded linear DNA (linear) were incubated in 70% serum for 1 h. The percentage of intact DNA was measured by qPCR. (c) A known concentration of breast cancer cells (SKBR3) was serially diluted, cell number validated, and STAMP measurements were performed for the cytoplasmic protein marker CK19. Dotted line: limit of detection, defined as 3× s.d. of no-cell control. All measurements were performed in triplicates, and the data are presented as mean t s.d. (***P<0.0005, Student's t-test). a.u., arbitrary unit.

FIG. 13. STAMP measurements of protein expression and subcellular distribution (a) Schematic representation of the STAMP localization assay; (b) Subcellular distribution of the localization labels (L1, L2 and L3); (c) STAMP localization of markers of interest; and (d) STAMP measurements of marker expression and subcellular distribution. All signals were normalized with respective IgG isotype control signals. All measurements were performed in triplicates, and the data are presented as mean in b and as mean±s.d. in c and d.

FIG. 14. STAMP localization assay using mesoporous silica nanoparticles. Transmission electron microscopy (TEM) images of (a) small and (b) large mesoporous silica nanoparticles (MSNs) used for differential subcellular labeling. The small and large particles showed a mean diameter of ˜40 nm and 250 nm, respectively. (c) Subcellular distributions of the localization signals under different cell permeabilization strategies. (d) Subcellular distributions of the localizations signals in SKOV3 cells permeabilized with 0.1% Triton X-100, presented as a bar graph. All fluorescence measurements were normalized against respective marker expressions. All measurements were performed in triplicates, and the data are presented as mean t s.d.

FIG. 15. Immunofluorescence images of the localization signals and DNA nanostructures. MCF7 cells were labeled with antibodies against position markers (sodium-potassium ATPase for plasma membrane, α-tubulin for cytoplasm, and histone H2B for nucleus), and targeted with (a) fluorescent localization signals (attached onto different MSNs) and (b) fluorescent DNA nanostructures. All scale bars: 25 μm.

FIG. 16. Steps in STAMP data processing for subcellular distribution analysis using a matrix.

FIG. 17. Microscopy analysis of target markers with different subcellular localizations. Single-channel and merged microscopy images of SKOV3 cells, immunostained with antibodies against markers of interest: plasma membrane marker (HER2), cytoplasmic marker (CK19), and nuclear protein (histone H3). The cells were also counterstained with nuclear dye Hoechst 33342. (a) Fixed cells were permeabilized with 0.1% Triton X-100 before immunostaining. (b) Live cells were permeabilized in 0.1% saponin for immunostaining. Note for live-cell imaging the nuclear histone H3 could not be targeted with this approach as saponin does not permeabilize the nuclear envelope. The analysis confirmed the subcellular localizations of the markers. All scale bars: 50 μm.

FIG. 18. Amplification efficiencies of STAMP barcodes. qPCR calibration curves of STAMP barcodes with their respective primer sets. The goodness of fit (R²), slope, and PCR efficiency calculated from the slope are presented for each barcode. All sets show an efficiency >87%. All measurements were performed in triplicates, and the data are presented as mean±s.d. (error bars mostly not visible). Sequences of the barcodes (e.g., Tetrahedron1-L1, Tetrahedron1-L2) and their respective primer sets are presented in Table 3.

FIG. 19. Specificity of STAMP primers. Each STAMP barcode was subjected to qPCR analysis with all primer sets to examine the signal specificity. All qPCR signals were globally normalized (i.e., the highest signal obtained was normalized as 100%). No significant crosstalk was observed between the primer sets. Sequences of the barcodes (e.g., Tetrahedron1-L1, Tetrahedron1-L2) and their respective primer sets are presented in Table 3.

FIG. 20. Multiplexed STAMP for high-throughput cellular profiling. All protein measurements were performed by (a) multiplex STAMP, through simultaneous STAMP barcode generation and next generation sequencing analysis, and (b) singleplex flow cytometry, where fluorescent antibody measurements were made one at a time. All measurements were performed in triplicates, and normalized against respective IgG isotype controls. The data are presented as mean values. MFI, mean fluorescence intensity.

FIG. 21. STAMP analysis with next generation sequencing and qPCR. STAMP barcodes were generated from multiplexed cell line profiling and analyzed through either next generation sequencing or qPCR. Both results showed an excellent correlation to each other, and demonstrated good concordance to gold standard measurements, as determined by singleplex flow cytometry using the same antibodies. All signals were normalized against IgG isotype control antibodies. Measurements were performed in triplicates, and the data are presented as mean values. MFI, mean fluorescence intensity.

FIG. 22. Protein typing of rare clinical samples (a) Multiplexed STAMP analysis of protein markers in clinical samples; (b) Receiver operator characteristic (ROC) curves of the STAMP regression model on clinical specimens used for training the model (n=34, AUC=0.9715) and additional validation cohort (n=35, AUC=0.9406); (c) Cancer samples (n=35) were classified into three molecular subtypes (luminal, non-luminal HER2-positive, and triple-negative) based on their clinical pathology classification; (d) Subcellular protein distribution in cancer samples. All measurements were performed in triplicates, and normalized against respective IgG isotype controls. The data are presented as mean values. AUC, area under curve.

FIG. 23. Analyses of clinical samples. (a) Regression coefficients. (b) Receiver operator characteristic (ROC) curve of the STAMP regression model. (c) Numerical values of STAMP clinical measurements, as presented in FIG. 22c. (d) Comparison of clinical ER, PR, and HER2 amplification status with STAMP results. Clinical amplification status was determined from immunohistochemistry of surgical tissues. STAMP measurements were performed on patient-matched FNA samples. AUC, area under curve.

### DETAILED DESCRIPTION

Particular embodiments of the present invention will now be described with reference to the accompanying drawings. The terminology used herein is for the purpose of describing particular embodiments only and is not intended to limit the scope of the present invention. Additionally, unless defined otherwise, all technical and scientific terms used herein have the same meanings as commonly understood by one or ordinary skill in the art to which the present invention belongs. Where possible, the same reference numerals are used throughout the figures for clarity and consistency.

Various embodiments relate to a method for detecting and/or identifying a target in a sample comprising: (a) forming a modified moiety by conjugating a nucleic acid sequence to a moiety directed to the target; (b) forming a nucleic acid nanostructure comprising a segment sequence complementary to a portion of the nucleic acid sequence of the modified moiety; (c) incubating the sample with the modified moiety to form a complex between the modified moiety and the target; (d) removing modified moieties that do not form a complex with the target; (e) allowing the complementary segment sequence of the nanostructure to hybridize to the portion of the nucleic acid of the modified moiety to which it is complementary; (f) forming a nucleic acid barcode comprising the complementary segment sequence of the nanostructure; and (g) detecting the nucleic acid barcode, whereby the detection of the nucleic acid barcode indicates that the target protein is present in the sample.

As used herein the term ‘moiety’ refers to any molecule or compound that interacts with or binds to a biological compound with specificity and sensitivity. In various embodiments the moiety is an interacting moiety or a binding moiety. In various embodiments the moiety or interacting moiety or binding moiety interacts or binds with the target. In various embodiments the target is a biological compound selected from a protein, a lipid, and carbohydrates. In various embodiments the moiety is an antibody and the target is a target protein, wherein the antibody binds to the target protein with specificity and sensitivity. In various embodiments the target protein comprises a glycoprotein. In various embodiments the moiety is an organic moiety such as an organic molecule that interacts with or binds to a biological compound. In various embodiments, the moiety comprises a biologically active moiety. In various embodiments, the moiety comprises a therapeutically active agent that interacts or binds with a biological target.

In various embodiments, the nucleic acid may be deoxyribonucleic acid (DNA), modified DNA, ribonucleic acid (RNA), modified RNA, locked nucleic acid (LNA), peptide nucleic acids (PNA), threose nucleic acid (TNA), hexitol nucleic acid (HNA), bridge nucleic acid, cyclohexenyl nucleic acid, glycerol nucleic acid, morpholino, phosphomorpholino, aptamer and catalytic nucleic acid versions thereof. In various embodiments, the nucleic acid may comprise a nitrogenous base or a modified nitrogenous base such as, 2′-o-methyl DNA, 2′-o-methyl RNA, 2′-fluoroDNA, 2′-fluoro-RNA, 2′-methoxy-purine, 2′-fluoro-pyrimidine, 2′-methoxymethyl-DNA, 2-methoxymethyl-RNA, 2′-acrylamido-DNA, 2′-acrylamido-RNA, 2-ethanol-DNA, 2′-ethanol-RNA, 2′-methanol-DNA, 2′-methanol-RNA, and a combination thereof. In various embodiments, the nucleic acid may comprise a phosphate backbone or a modified phosphate backbone, such as a phosphorothioate backbone, phosphoroborate backbone, methyl phosphonate backbone, phosphoroselenoate backbone, or phosphoroamidate backbone. The advantage of using a nucleic acid nanostructure is that the nucleic acids of the nanostructure can form a portion of the barcode allowing smaller nucleic acid sequence to be used on both the antibody and the nucleic acid nanostructure to permit closer interaction and minimize steric hindrance.

In various embodiments the nucleic acid nanostructure is a DNA structure comprising discrete structures that have portions or segments that pair in non-Watson-Crick base pairing or in non-helical formation. The DNA nanostructures consist of high-density double stranded DNA providing the benefit of a stable condensed structure that is able to move close to the moiety such as an antibody in order to interact with the nucleic acid conjugated to the moiety such as an antibody. This facilitates improved DNA hybridization and/or ligation. In various embodiments the segment sequence complementary to a portion of the nucleic acid sequence of the modified moiety such as an antibody overhangs from the nucleic acid nanostructure.

As used herein the terms ‘conjugation’, ‘conjugating’ or ‘conjugated’ may refer to any chemistry known in the art capable of forming covalent bond or any other bond that securely attaches a nucleic acid sequence to the moiety, such as a nucleic acid sequence covalently bonded to an antibody.

As used herein the term sample refers to a cell sample. In various embodiments the cell sample may be a blood sample a biopsy sample including a fine needle aspiration biopsy (FNA biopsies), urine samples, stool samples, saliva samples, tear samples or any sample containing cells. In various embodiments the sample may be a cancer sample such as a breast cancer sample.

In various embodiments the nucleic acid nanostructure comprises any shape including cube shaped, tetrahedron, octahedron or any polyhedron or any irregular shapes made by methods known in the art provided the nanostructure is compact in order to have a close interaction with the target. In various embodiments the nucleic acid nanostructure is a tetrahedron. In various embodiments the tetrahedron is produced from 4 DNA strands where each edge of the tetrahedron is a 20 base pair DNA double helix and each vertex is a three arm junction, wherein the segment sequence complementary to a portion of the nucleic acid sequence of the modified antibody is part of a nucleic acid sequence that extends from or overhangs each vertex.

In various embodiments the nucleic acid sequence conjugated to the modified moiety is 50 nucleotides or less. In various embodiments the nucleic acid sequence is 50, 40, 37, 36, 35, 30, 29, 28, 27, 26, 25, 24, 23, 22, 21, 20, 19, 18, 17, 16, or 15 nucleotides in length. In various embodiments the nucleic acid sequence conjugated to the modified moiety is between 50 and 12 nucleotides in length, or between 40 and 20 nucleotides in length, or 37 to 27 nucleotides in length. The use of short DNA labels of 50 nucleotides or less conjugated to a moiety, such as an antibody, not only preserves the moiety activity, such as antibody performance, and enables cellular targeting in various subcellular compartments, but it also eases the moiety selection as any off-the-shelf moiety, including an off the shelf antibody can be used. Additionally, any conjugation chemistry, such as standard coupling such as NHSmaleimide, can be applied to any off-the-shelf moiety, such as any off-the-shelf antibody. Activating agents are commonly used in bioconjugate chemistry and are known in the art. In various embodiments, the at least one activating agent may be a carbodiimide (such as N,N′-dicyclohexylcarbodiimide (DCC), N,N′-dicyclopentylcarbodiimide, N,N′-diisopropylcarbodiimide (DIC), I-ethyl-3-(3-dimethylaminopropyl)carbodiimide (EDC)), an anhydride (such as a symmetric, mixed, or cyclic anhydride), an activated ester (such as phenyl activated ester derivatives, p-hydroxamic activated ester, hexafluoroacetone (HFA)), an acylazole (such as acylimidazoles using CDI, acylbenzotriazoles), an acyl azide, an acid halide, a phosphonium salt (such as HOBt, PyBOP, HO At), an aminium/uronium salt (such as tetramethyl aminium salts, bispyrrolidino aminium salts, bispiperidino aminium salts, imidazolium uronium salts, pyrimidinium uronium salts, uronium salts derived from N,N,N′-trimethyl-N′-phenylurea, morpholino-based aminium/uronium coupling reagents, antimoniate uronium salts), an organophosphorus reagent (such as phosphinic and phosphoric acid derivatives), an organosulfur reagent (such as sulfonic acid derivatives), a triazine coupling reagent (such as 2-chloro-4,6-dimethoxy-1,3,5-triazine, 4-(4,6-dimethoxy-1,3,5-triazin-2-yl)-4 methylmorpholinium chloride, 4-(4,6-dimethoxy-1,3,5-triazin-2-yl)-4 methylmorpholinium tetrafluoroborate), a pyridinium coupling reagent (such as pyridinium tetrafluoroborate coupling reagents), a polymer-supported reagent (such as polymer-bound carbodiimide, polymer-bound TBTU, polymer-bound 2,4,6-trichloro-1,3,5-triazine, polymer-bound HOBt, polymer-bound HOSu, polymer-bound IIDQ, polymer-bound EEDQ).

In various embodiments a plurality of modified moieties directed to a plurality of targets and a plurality of nucleic acid nanostructures comprising a segment sequence complementary to a portion of each of the plurality of nucleic acid sequences of the modified moieties are formed. In various embodiments the plurality of modified moieties refers to 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 50 or more, 100 or more. A person skilled in the art would understand that it is possible to use any number of moieties simultaneously with the methods described herein. In various embodiments minor variations in the nucleic acid sequence conjugated to each moiety make it possible to identify the moiety via sequencing of the subsequently generated barcode. This permits detection and identification of multiple targets at one time. In various embodiments the target is a target protein and the target protein comprises any one of sodium-potassium ATPase; alpha-tubulin; Histone H2B; HER2; CK19; Histone H3; CD44; S100P; EpCAM; CA125; CD24; TSPAN8; ER; PR; CD9; VEGFR; EGFR; CD45; CD41 or a combination thereof.

Each moiety directed to a target to which it binds has a specific and unique nucleic acid sequence conjugated thereto. In various embodiments the nucleic acid sequence conjugated to a first moiety differs from a nucleic acid sequence conjugated to a second moiety by only 1 or 2 nucleotides. The greater the differences between the nucleotides the easier it will be to determine the difference between each specific and unique nucleic acid sequence which is associated with each unique moiety directed to each target. This will result in the formation of a plurality of unique barcodes comprising the complementary segment sequence of the nanostructure that is complementary to a portion of the nucleic acid sequence capable of being conjugated to each moiety directed to each target.

In various embodiments the method further comprises forming at least two different location identifiers wherein each location identifier comprises a nucleic acid sequence comprising a segment sequence complementary to a second portion of the nucleic acid sequence of the modified moiety and a unique identifier that can be used to determine the cell or subcellular location of the target when the location identifiers bind to the second portion of the nucleic acid sequence of the modified moiety.

In various embodiments the at least two different location identifiers may comprise 2, 3, 4, 5 or more different location identifiers each being directed to a specific cellular or subcellular location. In various embodiments the location identifiers may be directed to a specific cellular or subcellular location by the size of the unique identifier allowing the location identifier to either pass through a cell membrane or not or pass through a nuclear envelop or not. In various embodiments the location identifiers may be directed to a specific cellular or subcellular location by adjusting the pore size of cell membranes or nuclear envelop in different samples to allow the location identifier to either pass through a cell membrane or not or pass through a nuclear envelop or not. It would be appreciated by a person skilled in the art that the pore size of a cell membrane or the nuclear envelop can be adjusted by differentially permeabilizing the cell membrane or nuclear envelop via means such as electroporation or chemically such as by adjusting polarity.

In various embodiments the unique identifier may be any means to direct the at least two different location identifiers to a cellular location or subcellular location such as a targeting moiety, an antibody or any other known method of directing nucleic acid to a particular cellular location or subcellular location.

In various embodiments the unique identifier comprises a nucleic acid sequence.

In various embodiments the method further comprising permeabilizing a first cell sample with a first permeabilization buffer to allow a first location identifier to enter a first subcellular location of the cell and permeabilizing a second cell sample with a second permeabilization buffer to allow a second location identifier to enter a second subcellular location of the cell wherein the first and second subcellular location are not the same and detection of the first, or second location identifier indicates the amount of the target present in different subcellular locations.

In various embodiments each of the at least two different location identifiers may be directed to a particular cellular or subcellular location. In various embodiments each of the at least two different location identifiers may be directed to any one of a particular organ, a cell membrane, a cytoplasm, a nucleus, a subcellular organelle such as a vacuole, an endoplasmic reticulum, a golgi apparatus, a mitochondria of any other subcellular compartment known to a person skilled in the art.

In various embodiments the at least two different location identifiers may comprise three separate location identifiers, a first location identifier comprising a nucleic acid sequence comprising a segment sequence complementary to a second portion of the nucleic acid sequence of the modified moiety wherein the unique identifier comprises a nucleic acid sequence, a second location identifier comprising a nucleic acid sequence comprising a segment sequence complementary to a second portion of the nucleic acid sequence of the modified moiety conjugated to a nanoparticle having a diameter of 60 nm or less wherein the unique identifier of the second location identifier comprises a nucleic acid sequence in combination with the conjugated to a nanoparticle having a diameter of 60 nm or less; and a third location identifier comprising a nucleic acid sequence comprising a segment sequence complementary to a second portion of the nucleic acid sequence of the modified moiety conjugated to a nanoparticle having a diameter of 100 nm or more wherein the unique identifier of the third location identifier comprises a nucleic acid sequence in combination with the conjugated to a nanoparticle having a diameter of 100 nm or more; wherein detection of the first second or third location identifier indicates where in the cellular environment the target was present.

In various embodiments detection of the third location identifier indicates the target was present on the cell membrane, detection of the second location identifier indicates the target was present outside the nucleus, or detection of the first location identifier indicates the target was present at any location within the cell or cellular environment.

In various embodiments the second location identifier comprises a diameter less than 60 nm. In various embodiments the second location identifier comprises a diameter between 60 nm and 10 nm. In various embodiments the second location identifier comprises a diameter of 20 nm, or 30 nm, or 40 nm, or 50 nm. In various embodiments the nanoparticle of the second location identifier comprises mesoporous silica nanoparticle.

In various embodiments the third location identifier comprises a diameter of 100 nm or more. In various embodiments the second location identifier comprises a diameter between 100 nm and 300 nm. In various embodiments the second location identifier comprises a diameter of 150 nm, or 200 nm, or 250 nm. In various embodiments the nanoparticle of the third location identifier comprises mesoporous silica nanoparticle.

It would be appreciated by a person skilled in the art that the first location identifier would be able to pass through the cell membrane and the nuclear envelope and as such may be present in the nucleus, the cytoplasm or the external cellular environment including on the cell surface. As such it would be able to interact with the modified antibodies anywhere within the cell or the cellular environment. Similarly, it would be appreciated by a person skilled in the art that the second location identifier would be able to pass through the cell membrane but may not be able to pass through the nuclear envelope and as such may be present in the cytoplasm or the external cellular environment including on the cell surface but may not be present the nucleus. As such it would be able to interact with the modified antibodies anywhere within the cytoplasm or the cellular environment. Further, it would be appreciated by a person skilled in the art that the third location identifier would not be able to pass through the cell membrane or the nuclear envelope and as such may be present in the external cellular environment including on the cell surface but may not be present in the cytoplasm or the nucleus. As such it would only be able to interact with the modified antibodies present outside the cell.

In various embodiments the method further comprising ligating the complementary segment sequence of the nanostructure with the complementary segment sequence of the first, second or third location identifiers; forming a nucleic acid barcode comprising the ligated complementary segment sequence of the nanostructure and the complementary segment sequence of the first, second or third location identifiers. In various embodiments the complementary segment sequence of the nanostructure hybridizes to a first portion of the nucleic acid conjugated to the moiety and the complementary segment sequence of the first, second or third location identifiers hybridizes to a second portion of the nucleic acid conjugated to the moiety wherein the first and second portion are in close proximity facilitating ligation between the complementary strands i.e. the complementary segment sequence of the nanostructure and the complementary segment sequence of the first, second or third location identifiers.

In various embodiments the method further comprising: determining the relative location of at least two reference target proteins, each having a different known cellular distribution, with a set of interacting nucleic acid structures comprising i) a modified antibody having a nucleic acid sequence conjugated thereto directed to each reference target protein; ii) a nucleic acid nanostructure comprising a segment sequence complementary to a portion of the nucleic acid sequence conjugated to the modified antibody; and iii) a unique identifier by detecting at least two reference barcodes formed comprising the complementary segment sequence of the nanostructure, the complementary segment sequence of the second portion of the nucleic acid sequence of the modified antibody and a unique identifier; determining the relative amount and/or location of at least one target proteins with a set of interacting nucleic acid structures comprising i) a modified moiety having a nucleic acid sequence conjugated thereto directed to each target protein; ii) a nucleic acid nanostructure comprising a segment sequence complementary to a portion of the nucleic acid sequence conjugated to the modified moiety; and iii) a unique identifier, by detecting at least one barcode comprising the complementary segment sequence of the nanostructure, the complementary segment sequence of the second portion of the nucleic acid sequence of the modified moiety and a unique identifier; analyzing the at least two reference barcodes formed when the reference target protein is present and comparing the relative distribution of the at least two reference barcodes with the at least one barcode formed when the target protein is present and determining the relative cellular location of the target protein.

In various embodiments the method further comprising: determining the amount of at least two reference target proteins, each having a different known cellular distribution with a set of interacting nucleic acid structures comprising a modified antibody having a nucleic acid sequence conjugated thereto directed to each reference target protein, the nucleic acid nanostructure comprising a segment sequence complementary to a portion of the nucleic acid sequence conjugated to each modified antibody directed to each reference target protein; by detecting at least two reference barcodes formed comprising the complementary segment sequence of the nanostructure, the complementary segment sequence of the second portion of the nucleic acid sequence of the modified antibody and a unique identifier; determining the amount of at least two target proteins with a set of interacting nucleic acid structures comprising a modified moiety having a nucleic acid sequence conjugated thereto directed to each target protein; a nucleic acid nanostructure comprising a segment sequence complementary to a portion of the nucleic acid sequence conjugated to the modified moiety by detecting; at least two barcodes comprising the complementary segment sequence of the nanostructure, the complementary segment sequence of the second portion of the nucleic acid sequence of the modified moiety and a unique identifier; analyzing the at least two reference barcodes formed when the reference target protein is present and comparing the relative distribution of the at least two reference barcodes with the at least two barcodes formed when the target protein is present and determining the relative cellular location of the target protein.

In various embodiments the relative cellular location of the target protein is determined via a matrix conversion, however, any 3 dimensional modeling analysis known in the art capable of mapping the cellular or subcellular location of the target protein relative to the distribution of a known or predicted cellular location of a reference target protein would be suitable to determine the cellular or subcellular location of the target protein.

In various embodiments the reference target protein having a known cellular location are position markers or proteins associated primarily or only with a specific location in a cellular environment including a physiological environment. In various embodiments the reference target protein having a known cellular location may be a protein unique to a particular organ or tissue. Generally each tissue has about 1,000 proteins uniquely expressed in that tissue alone and not detected in any other tissue. In various embodiments the tissue specific protein comprises a receptor antigen with an elevated expression in one tissue compared to other tissue types. In various embodiments the tissue specific protein comprises a liver specific protein, a kidney specific protein, a heart specific protein, a lung specific protein, a pancreas specific protein, an intestine specific protein or a thymus specific protein. In various embodiments the tissue specific protein comprises bone specific protein, tendon specific protein, skin specific protein, nerve specific protein, vein specific protein, corneal specific protein. In various embodiments the reference target protein having a known cellular location may be a protein associated primarily or only with a plasma membrane such as sodium-potassium ATPase. In various embodiments the reference target protein having a known cellular location may be a protein associated primarily or only with the cytoplasm such as alpha tubulin. In various embodiments the reference target protein having a known cellular location may be a protein associated primarily or only with the nucleus such as histone H2B. In various embodiments the reference target protein having a known cellular location may be a protein associated primarily or only with any one of a cell membrane, a cytoplasm, a nucleus, a subcellular organelle such as a vacuole, an endoplasmic reticulum, a golgi apparatus, a mitochondria of any other subcellular compartment known to a person skilled in the art.

In various embodiments at least two reference target proteins are used to map the relative distribution of the target proteins based on the analysis of the at least two reference barcodes with the at least two barcodes formed when the target protein is present. In various embodiments a unity matrix is used to determine a conversion function that can be used to determine the relative distribution of the target associated with nucleic acid barcode detected.

In various embodiments the nanoparticle comprises mesoporous silica nanoparticle.

In various embodiment the ligation may be with blunt ends or sticky ends. In various embodiments the ligation is facilitated by enzymatic ligase. In various embodiments a DNA ligase is used such as a T4 DNA ligase.

In various embodiments the nucleic acid sequence nanostructure is further modified chemically at the 5′-end and the nucleic acid segment sequence of each location identifier complementary to a second portion of the nucleic acid sequence of the modified moiety is modified chemically at the 5′-end to facilitate ligation.

In various embodiments the nucleic acid sequence nanostructures further modified chemically at the 5′-end with azide and the nucleic acid segment sequence of each location identifier complementary to a second portion of the nucleic acid sequence of the modified moiety is modified chemically at the 5′-end with alkyne or hexynyl to facilitate ligation. In various embodiments the ligation is facilitated by copper sulfate.

In various embodiments where the moiety comprises an antibody, the antibody is modified with at least two nucleic acid sequence strands per antibody. In various embodiments the antibody is modified with between 2 and 5 nucleic acid strands per antibody. In various embodiments the antibody is modified with an average of 2.5 nucleic acid sequence strands per antibody.

In various embodiments the incubation is facilitated with a permeabilization buffer. This may assist the moiety to enter cells in the sample.

As all reference barcodes and the barcodes of the individual targets all comprise nucleic acid sequences they are all compatible and would be able to be detected with any PCR or next generation sequencing methods known in the art.

Another aspect of the invention relates to a set of interacting nucleic acid structures for use in detecting and/or identifying a target comprising: (a) a nucleic acid sequence capable of being conjugated to a moiety directed to the target; and (b) a nucleic acid nanostructure comprising a segment sequence complementary to a portion of the nucleic acid sequence capable of being conjugated to an moiety directed to the target.

In various embodiments the nucleic acid sequence is capable of being conjugated to a moiety directed to the target by being modified with a sulfhydryl group such as a thiol. Activating agents are commonly used in conjugate chemistry and are known in the art. In various embodiments, the at least one activating agent may be a carbodiimide (such as N,N′-dicyclohexylcarbodiimide (DCC), N,N′-dicyclopentylcarbodiimide, N,N′-diisopropylcarbodiimide (DIC), I-ethyl-3-(3-dimethylaminopropyl)carbodiimide (EDC)), an anhydride (such as a symmetric, mixed, or cyclic anhydride), an activated ester (such as phenyl activated ester derivatives, p-hydroxamic activated ester, hexafluoroacetone (HFA)), an acylazole (such as acylimidazoles using CDI, acylbenzotriazoles), an acyl azide, an acid halide, a phosphonium salt (such as HOBt, PyBOP, HO At), an aminium/uronium salt (such as tetramethyl aminium salts, bispyrrolidino aminium salts, bispiperidino aminium salts, imidazolium uronium salts, pyrimidinium uronium salts, uronium salts derived from N,N,N′-trimethyl-N′-phenylurea, morpholino-based aminium/uronium coupling reagents, antimoniate uronium salts), an organophosphorus reagent (such as phosphinic and phosphoric acid derivatives), an organosulfur reagent (such as sulfonic acid derivatives), a triazine coupling reagent (such as 2-chloro-4,6-dimethoxy-1,3,5-triazine, 4-(4,6-dimethoxy-1,3,5-triazin-2-yl)-4 methylmorpholinium chloride, 4-(4,6-dimethoxy-1,3,5-triazin-2-yl)-4 methylmorpholinium tetrafluoroborate), a pyridinium coupling reagent (such as pyridinium tetrafluoroborate coupling reagents), a polymer-supported reagent (such as polymer-bound carbodiimide, polymer-bound TBTU, polymer-bound 2,4,6-trichloro-1,3,5-triazine, polymer-bound HOBt, polymer-bound HOSu, polymer-bound IIDQ, polymer-bound EEDQ).

In various embodiments the segment sequence complementary to the portion of the nucleic acid sequence comprises at least half the nucleic acid sequence capable of being conjugated to the moiety directed to the target. In various embodiment not all the bases of the segment sequence complementary to a portion of the nucleic acid sequence are complementary, at least half the nucleic acid sequence capable of being conjugated to moiety directed to the target are complementary. In various embodiments at least 60%, 70%, 80%, 90%, 95%, 96%, 97%, 98%, 99% of the bases of the segment sequence complementary to a portion of the nucleic acid sequence are complementary. In various embodiments all the bases of the segment sequence complementary to a portion of the nucleic acid sequence are complementary.

In various embodiments the nucleic acid nanostructure comprises any shape including cube shaped, tetrahedron, octahedron or any polyhedron or any irregular shape made by methods known in the art provided the nanostructure is compact enough to have a close interaction with the target. In various embodiments the nucleic acid nanostructure is a tetrahedron. In various embodiments the nucleic acid nanostructure is formed from SEQ ID NOS. 3 to 7. In various embodiments the nucleic acid nanostructure is formed from SEQ ID NOS. 8 to 9.

In various embodiments the nucleic acid sequence capable of being conjugated to a moiety is 50 nucleotides or less. In various embodiments the nucleic acid sequence capable of being conjugated to a moiety is 50, 40, 37, 36, 35, 30, 29, 28, 27, 26, 25, 24, 23, 22, 21, 20, 19, 18, 17, 16, or 15 nucleotides in length. In various embodiments the nucleic acid sequence capable of being conjugated to a moiety is between 50 and 12 nucleotides in length, or between 40 and 20 nucleotides in length, or 37 to 27 nucleotides in length. In various embodiments the moiety that the nucleic acid sequence is capable of being conjugated to is an antibody, such short nucleic acid sequences will not interfere with the antibody function resulting in minimal loss of specificity and sensitivity as there is minimal interference from the nucleic acid sequence.

In various embodiments the nucleic acid sequence capable of being conjugated to a moiety such as an antibody directed to the target comprises SEQ ID NO. 1 and the segment sequence complementary to a portion of the nucleic acid sequence comprises SEQ ID NO. 90 (SEQ ID NO. 90—AAGTATCATACCCGT) or SEQ ID NO. 7 or SEQ ID NO. 9. In various embodiment not all the bases of the segment sequence complementary to a portion of the nucleic acid sequence are complementary, at least 15 consecutive base pairs of the segment sequence complementary to a portion of the nucleic acid sequence are complementary. This allows the ends to interact and hybridize and facilitate hybridization.

In various embodiments the set further comprises at least two different location identifiers wherein each location identifier comprises a nucleic acid sequence comprising a segment sequence complementary to a second portion of the nucleic acid sequence of the modified moiety and a unique identifier.

In various embodiments the nucleic acid sequence comprising a segment sequence complementary to a second portion of the nucleic acid sequence of the modified moiety comprises SEQ ID NO. 91 (SEQ ID NO. 91—ATCATA) or SEQ ID NO. 92 (SEQ ID NO. 92—AAGTATCATACCCGT).

In various embodiments the unique identifier comprises a nucleic acid sequence. In various embodiments the unique identifier comprises any one of SEQ ID NO. 93 (SEQ ID NO. 93—CTCCACGACTTAGAATC), SEQ ID NO. 94 (SEQ ID NO. 94—AGTTGCTGGACGATTGT), SEQ ID NO. 95 (SEQ ID NO. 95—TCACCGTAGCTCAATGG),

In various embodiments the at least two different location identifiers comprise at least three separate location identifiers, a first location identifier comprising a nucleic acid sequence comprising a segment sequence complementary to a second portion of the nucleic acid sequence of the modified moiety wherein the unique identifier comprises a nucleic acid sequence, a second location identifier comprising a nucleic acid sequence comprising a segment sequence complementary to a second portion of the nucleic acid sequence of the modified moiety conjugated to a nanoparticle having a diameter of 60 nm or less wherein the unique identifier of the second location identifier comprises a nucleic acid sequence in combination with the conjugated to a nanoparticle having a diameter of 60 nm or less; and a third location identifier comprising a nucleic acid sequence comprising a segment sequence complementary to a second portion of the nucleic acid sequence of the modified moiety conjugated to a nanoparticle having a diameter of 100 nm or more wherein the unique identifier of the third location identifier comprises a nucleic acid sequence in combination with the conjugated to a nanoparticle having a diameter of 100 nm or more.

In various embodiments the first location identifier comprises SEQ ID NO. 16 wherein the nucleic acid sequence comprising a segment sequence complementary to a second portion of the nucleic acid sequence of the modified moiety comprises SEQ ID NO. 91 or SEQ ID NO. 92 and the unique identifier comprises SEQ ID NO. 93.

In various embodiments the second location identifier comprises SEQ ID NO. 17 wherein the nucleic acid sequence comprising a segment sequence complementary to a second portion of the nucleic acid sequence of the modified moiety comprises SEQ ID NO. 91 or SEQ ID NO. 92 and the unique identifier comprises SEQ ID NO. 94 in combination with the conjugated to a nanoparticle having a diameter of 60 nm or less.

In various embodiments the second location identifier comprises a diameter less than 60 nm. In various embodiments the second location identifier comprises a diameter between 60 nm and 10 nm. In various embodiments the second location identifier comprises a diameter of 20 nm, or 30 nm, or 40 nm, or 50 nm. In various embodiments the nanoparticle of the second location identifier comprises mesoporous silica nanoparticle.

In various embodiments the third location identifier comprises SEQ ID NO. 18 wherein the nucleic acid sequence comprising a segment sequence complementary to a second portion of the nucleic acid sequence of the modified moiety comprises SEQ ID NO. 91 or SEQ ID NO. 92 and the unique identifier comprises SEQ ID NO. 95 in combination with the conjugated to a nanoparticle having a diameter of 100 nm or more.

In various embodiments the third location identifier comprises a diameter of 100 nm or more. In various embodiments the second location identifier comprises a diameter between 100 nm and 300 nm. In various embodiments the second location identifier comprises a diameter of 150 nm, or 200 nm, or 250 nm. In various embodiments the nanoparticle of the third location identifier comprises mesoporous silica nanoparticle.

In various embodiments the set further comprises at least two modified antibodies directed to at least two reference target protein having a known cellular location.

In various embodiments the nucleic acid sequence capable of being conjugated to a moiety is conjugated to a moiety directed to the target.

In various embodiments the moiety comprises an antibody and the target is a protein target.

Various embodiments relate to a system for detection and/or identification of a target in a sample comprising: (a) a mixing chamber for mixing the sample with a modified moiety comprising a nucleic acid sequence conjugating to a moiety directed to the target; (b) a filter for capturing a complex between the modified moiety and the target; (c) a reservoir for incubating the moiety complex with a nucleic acid nanostructure comprising a segment sequence complementary to a portion of the nucleic acid sequence of the modified moiety and optionally at least two location identifiers; and (d) a detection chamber for detecting a nucleic acid barcode comprising the segment sequence complementary to the portion of the nucleic acid of the modified moiety.

In various embodiments the filter comprises a porous membrane.

In various embodiments the detection chamber comprises a plurality of detection chambers for detection of a plurality of barcodes indicative of a plurality of target proteins.

In various embodiments the system further comprises a heating element. In various embodiments the heat element can be heated to a temperature of 50° C. or above, preferably 65° C. facilitating linearization of the nucleic acid and liberation of barcodes.

In various embodiments the system further comprising a set of interacting nucleic acid structure as described in any of the embodiments herein above.

Various embodiments relate to a method of diagnosing a disease comprising: (a) forming a modified antibody by conjugating a nucleic acid sequence to an antibody directed to a target protein associated with the disease; (b) forming a nucleic acid nanostructure comprising a segment sequence complementary to a portion of the nucleic acid sequence of the modified antibody; (c) forming at least two different location identifiers, wherein each location identifier comprises a nucleic acid sequence comprising a segment sequence complementary to a second portion of the nucleic acid sequence of the modified antibody and a unique identifier; (d) incubating a sample with the modified antibody to form a complex between the modified antibody and the target protein, (e) removing any modified antibody that does not form a complex with the target protein; (f) incubating the complex with the nucleic acid nanostructure, and at least one location identifier to form a super complex between the modified antibody, the target protein and the location identifier; (g) ligating the nucleic acid of the super-complex between the complementary segment sequence of the nucleic acid nanostructure and the complementary segment sequence of the location identifier; (h) forming a nucleic acid barcode comprising the ligated sequence complementary to the portion of the nucleic acid sequence of the modified antibody and the sequence complementary to the second portion of the nucleic acid sequence of the modified antibody; and (i) detecting and analyzing the nucleic acid barcodes to determine the amount and/or subcellular distribution of target proteins, whereby the amount and/or subcellular distribution of target protein indicates the disease.

In various embodiments the method of diagnosing a disease subtype comprises: (a) forming a modified antibody by conjugating a nucleic acid sequence to an antibody directed to a target protein associated with the disease; (b) forming a nucleic acid nanostructure comprising a segment sequence complementary to a portion of the nucleic acid sequence of the modified antibody; (c) forming at least two different location identifiers, wherein each location identifier comprises a nucleic acid sequence comprising a segment sequence complementary to a second portion of the nucleic acid sequence of the modified antibody and a unique identifier; (d) incubating a sample with the modified antibody to form a complex between the modified antibody and the target protein, (e) removing any modified antibody that does not form a complex with the target protein; (f) incubating the complex between the modified antibody and the target protein with the nucleic acid nanostructures and at least one location identifier to form a super complex between the modified antibody, the target protein and the at least one location identifier, (g) ligating the nucleic acid of the super-complex with the segment sequence complementary to the portion of the nucleic acid nanostructure to the complementary segment sequence of the location identifier; (h) forming a nucleic acid barcode comprising the ligated sequence complementary to the portion of the nucleic acid sequence of the modified antibody and the sequence complementary to the second portion of the nucleic acid sequence of the modified antibody; and (i) detecting the nucleic acid barcode to determine the cellular location from the unique identifier, whereby the cellular location of the target protein indicates the disease subtype.

In various embodiments the at least one location identifier comprises at least two different location identifiers wherein each location identifier comprises a nucleic acid sequence comprising a segment sequence complementary to a second portion of the nucleic acid sequence of the modified moiety and a unique identifier that can be used to determine the cell or subcellular location of the target when the location identifiers bind to the second portion of the nucleic acid sequence of the modified moiety.

In various embodiments the at least two different location identifiers may comprise 2, 3, 4, 5 or more different location identifiers each being directed to a specific cellular or subcellular location. In various embodiments the location identifiers may be directed to a specific cellular or subcellular location by the size of the unique identifier allowing the location identifier to either pass through a cell membrane or not or pass through a nuclear envelop or not. In various embodiments the location identifiers may be directed to a specific cellular or subcellular location by adjusting the pore size of cell membranes or nuclear envelop in different samples to allow the location identifier to either pass through a cell membrane or not or pass through a nuclear envelop or not. It would be appreciated by a person skilled in the art that the pore size of a cell membrane or the nuclear envelop can be adjusted by differentially permeabilizing the cell membrane or nuclear envelop via means such as electroporation or chemically such as by adjusting polarity.

In various embodiments the unique identifier comprises a nucleic acid sequence. In various embodiments each of the at least two different location identifiers may be directed to a particular cellular or suboellular location. In various embodiments each of the at least two different location identifiers may be directed to any one of a particular organ, a cell membrane, a cytoplasm, a nucleus, a subcellular organelle such as a vacuole, an endoplasmic reticulum, a Golgi apparatus, a mitochondria of any other subcellular compartment known to a person skilled in the art.

In various embodiments the at least one location identifiers may comprise three separate location identifiers, a first location identifier comprising a first location identifier comprising a nucleic acid sequence comprising a segment sequence complementary to a portion of the nucleic acid sequence of the modified antibody, a second location identifier comprising a nucleic acid sequence comprising a segment sequence complementary to a portion of the nucleic acid sequence of the modified antibody conjugated to a nanoparticle having a diameter of 60 nm or less and a third location identifier comprising a nucleic acid sequence comprising a segment sequence complementary to a portion of the nucleic acid sequence of the modified antibody conjugated to a nanoparticle having a diameter of 100 nm or more.

In various embodiments the method further comprises: measuring at least two reference target proteins each having a known cellular location using a modified antibody directed to each reference target protein; by determining the amount of at least two reference target proteins, each having a different known cellular distribution, with a set of interacting nucleic acid structures comprising a modified antibody having a nucleic acid sequence conjugated thereto directed to each reference target protein; a nucleic acid nanostructure comprising a segment sequence complementary to a portion of the nucleic acid sequence conjugated to the modified antibody by detecting at least two reference barcodes formed comprising the complementary segment sequence of the nanostructure, the complementary segment sequence of the second portion of the nucleic acid sequence of the modified antibody and a unique identifier;

determining the relative amount of at least one target protein with a set of interacting nucleic acid structures comprising a modified moiety having a nucleic acid sequence conjugated thereto directed to each target protein; a nucleic acid nanostructure comprising a segment sequence complementary to a portion of the nucleic acid sequence conjugated to the modified moiety by detecting; at least two barcodes comprising the complementary segment sequence of the nanostructure, the complementary segment sequence of the second portion of the nucleic acid sequence of the modified moiety and a unique identifier; analyzing the at least two reference barcodes formed when the reference target protein is present and comparing the relative distribution of the at least two reference barcodes with the barcode formed when the target protein is present and determining the relative cellular location of the target protein.

In various embodiments the relative cellular location of the target protein is determined via a matrix conversion as described herein. However, any 3 dimensional modeling analysis known in the art capable of mapping the cellular or subcellular location of the target protein relative to the distribution of a known or predicted cellular location of a reference target protein would be suitable to determine the cellular or subcellular location of the target protein.

In various embodiments the disease is a disease subtype.

In various embodiments the disease subtype is a cancer subtype. In various embodiments the disease subtype is an aggressive cancer subtype.

In various embodiments the disease subtype is breast cancer. In various embodiments the cancer subtype is breast cancer. In various embodiments he aggressive cancer subtype is breast cancer. In various embodiments an aggressive cancer subtype refers to a cancer that has metastasized. In various embodiments an aggressive cancer subtype refers to a cancer that has poor long term survival or is likely to recur as may be determined via a Kaplan-Meier estimator.

In various embodiments the disease subtype is a cancer subtype. In various embodiments the cancer subtype comprises an aggressive cancer subtype. In various embodiments the cancer subtype is a cancer associated with tumorigenic proteins that are sequestered to the nucleus when they are phosphorylated. In various embodiments the cancer is breast cancer. In various embodiments tumorigenic proteins that are sequestered to the nucleus when they are phosphorylated comprise any one of estrogen receptor (ER), progesterone receptor (PR), human epidermal growth factor receptor 2 (HER2), and WW domain binding protein 2 (WBP2).

In various embodiments detection of more of the nucleic acid barcode comprising a unique identifier located in the nucleus compared to the nucleic acid barcode comprising a unique identifier located in other subcellular locations indicates the breast cancer is aggressive.

In various embodiments the target protein associated with breast cancer is selected from the group consisting of ER, PR, HER2 and a combination thereof.

In various embodiments the breast cancer is subtyped into luminal, non-luminal or triple negative based the expression of ER, PR and HER2, wherein detection of the nucleic acid barcode comprising the segment sequence complementary to the portion of the nucleic acid sequence of the modified antibody directed to the ER, and/or detection of the nucleic acid barcode comprising the segment sequence complementary to the portion of the nucleic acid sequence of the modified antibody directed to the PR, and/or detection of the nucleic acid barcode comprising the segment sequence complementary to the portion of the nucleic acid sequence of the modified antibody directed to the HER2 indicates the breast cancer is a luminal subtype; or wherein detection of the nucleic acid barcode comprising the segment sequence complementary to the portion of the nucleic acid sequence of the modified antibody directed to HER2 but not the segment sequence complementary to the portion of the nucleic acid sequence of the modified antibody directed to ER or PR indicates the breast cancer is a non-luminal subtype; or wherein the absence of the nucleic acid barcode comprising the segment sequence complementary to the portion of the nucleic acid sequence of the modified antibody directed to the ER, PR and HER2 indicates the breast cancer is a triple negative subtype.

Throughout the specification, unless otherwise indicated to the contrary, the terms “comprising”, “consisting of”, and the like, are to be construed as non-exhaustive, or in other words, as meaning “including, but not limited to”.

Throughout the specification, unless the context requires otherwise, the word “comprise” or variations such as “comprises” or “comprising”, will be understood to imply the inclusion of a stated integer or group of integers but not the exclusion of any other integer or group of integers.

Throughout the specification, unless the context requires otherwise, the word “include” or variations such as “includes” or “including”, will be understood to imply the inclusion of a stated integer or group of integers but not the exclusion of any other integer or group of integers.

As used herein, the term “about” typically means +/−5% of the stated value, more typically +/−4% of the stated value, more typically +/−3% of the stated value, more typically +/−2% of the stated value, even more typically +/−1% of the stated value, and even more typically +/−0.5% of the stated value.

Here, the unique properties of nucleic acid nanostructures are exploited to develop a sensitive 3D barcoding platform for multiplexed profiling of subcellular protein expression and distribution. The technology, termed DNA sequence-topology assembly for multiplexed profiling (DNA STAMP), utilizes the combinatorial sequence content of DNA nanostructures for diverse protein identification and their programmed structural conformation to significantly improve the reaction kinetics of enzymatic and chemical barcoding. Leveraging this sequence-structure synergy, in various embodiments DNA nanostructures are coupled with short localization labels. In various embodiments exogenous sequences which have differential distributions across subcellular compartments, are used to form multiplexed STAMP barcodes directly in whole cells. The generated DNA barcodes show improved sensitivity (>100-fold signal enhancement), and can reflect en masse protein expression and subcellular distribution in a high-throughput analysis.

When implemented on a miniaturized microfluidic device for clinical applications, STAMP enabled multiplexed protein typing and subcellular distribution analysis in scant patient samples. The STAMP-revealed signatures not only accurately classified cancer molecular subtypes, but also provided new measurements of disease aggressiveness.

Motivated by the DNA sequence-structure synergy, the STAMP technology was developed as a 3D barcoding platform. As compared to existing protein detection technologies, the STAMP platform shows distinct advantages, with respect to both assay format and assay performance.

STAMP is a solution-phase technology that enables both subcellular protein quantitation as well as localization analysis. It couples DNA nanostructures with localization labels to form multiplexed barcodes, which reflect both target protein expressions as well as their subcellular distributions. With respect to protein quantitation, STAMP is more sensitive than existing protein assays; it leverages compact DNA nanostructures to enhance all molecular reactions (e.g., antibody targeting, DNA hybridization, and barcode ligation), thereby improving its analytical sensitivity (10-22 mol) as well as capacity for detecting low-abundance proteins. With respect to subcellular localization analysis, STAMP utilizes molecular genetic approaches (e.g., PCR and sequencing) and matrix conversions to analyze the formed barcodes, thereby achieving high-throughput, multiplexed localization analysis.

Drawing on these advantages of 3D barcoding, STAMP is well-suited for sensitive and multiplexed protein typing. STAMP can be easily multiplexed to measure subcellular expression and localization of en masse proteins directly in whole cells, even in scant clinical samples. The STAMP-revealed signatures (i.e., subcellular expression and distribution) not only distinguish cancer molecular subtypes, but also reflect disease aggressiveness.

The scientific applications of the developed technology are potentially broad. With its enhanced barcoding efficiency and capacity, even in complex intracellular environments (e.g., cytoplasm and nucleus), the STAMP platform could be readily expanded to simultaneously detect, beyond proteins, other diverse molecules (e.g., RNAs, lipids and metabolites) of different subcellular localizations. In addition to measuring marker expression and subcellular distribution, the system could be further developed to quantify intracellular interactions and perform computational outputs, through the generation of STAMP barcodes from interacting DNA nanostructures. Technical improvements using different DNA nanostructures which demonstrate lock-and-key functionalities are likely to further enhance the barcoding kinetics to enable measurements of even transient and rare interactions.

Clinically, the STAMP technology could be applied to discover and establish comprehensive diagnostic and prognostic biomarker signatures. With its demonstrated robustness in rare patient specimens, STAMP barcodes could be generated from various clinical samples (e.g., tissue, blood, urine) across a spectrum of diseases (e.g., cancers, infectious diseases, neurodegenerative diseases) to develop composite signatures via high-throughput analyses. This could help to improve patient stratification and rationalize treatment decisions. Further microfluidic integration could accelerate large-scale clinical studies, by enabling diverse sample processing and highly parallel detection. Such use of DNA nanostructures as a universal barcoding material not only confers large capacity for information storage, but also benefits from many clinically available genomic platforms (e.g., PCR, sequencing) to facilitate parallel analysis of diverse targets and expedite large cohort clinical validations.

DNA possesses multidimensional information, beyond linear barcoding, to address current challenges in protein isolation, detection and/or identification. None Watson-Crick base pairing of nucleic acid was exploited to programmably fold nanostructures into 3D topologies. The unique properties of nucleic acid nanostructures was used to develop a highly sensitive 3D barcoding platform for multiplexed profiling of subcellular protein expression and distribution. Termed DNA sequence-topology assembly for multiplexed profiling (DNA STAMP), the technology utilizes the sequence content of DNA nanostructures to identify diverse proteins and their programmed structural conformation to significantly improve reaction kinetics at the molecular nanoscale. Specifically, the technology leverages the different organizational states of a configurable tetrahedral probe to complement every stage of DNA barcoding, thereby enhancing its analytical performance: 1) the compact assembly improves all relevant molecular reactions (i.e., antibody targeting, intracellular access and stability, DNA hybridization and barcode generation, via both enzymatic and chemical ligation approaches), and 2) the dissociated form fully extends to reveal its sequence content to enhance barcode differentiation.

Harnessing this sequence-structure synergy, the DNA nanostructures was coupled with localization labels, which are exogenous sequences having differential concentrations across subcellular compartments, to form multiplexed STAMP barcodes. These DNA barcodes are generated in situ from fixed cells, and reflect both protein identities as well as their subcellular distributions. Using different off-the-shelf antibodies, it was demonstrated that the STAMP technology not only show improved analytical signals to achieve single-cell detection sensitivity, but could also detect protein targets of various subcellular localizations (i.e., membrane, cytoplasmic and nuclear proteins) and map their distribution patterns in cells. All STAMP barcodes could be readily analyzed in a high throughput fashion, using established genomic platforms (e.g., qPCR and next generation sequencing). When implemented on a miniaturized microfluidic platform for clinical applications, STAMP enabled multiplexed protein typing of scant cells in patient breast fine needle aspirates. The STAMP signatures of subcellular protein expression and distribution not only accurately classified cancer molecular subtypes, but also revealed new measurements of disease aggressiveness.

Unless defined otherwise, all technical and scientific terms used herein have the same meaning as is commonly understood by a skilled person to which the subject matter herein belongs. As used herein, the following definitions are supplied in order to facilitate the understanding of the present invention.

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the specification, unless the contest requires otherwise, the word “include” or variations such as “includes” or “including” will be understood to imply the inclusion of a stated integer or group of integers but not the exclusion of any other integer or group of integers.

As used in the specification and the appended claims, the singular form “a”, and “the” include plural references unless the context clearly dictates otherwise.

### EXAMPLES

### Example 1: DNA STAMP Platform

Cell culture. SKBR3 cell line was obtained from American Type Culture Collection (ATCC). SKBR3 cell line was grown in Dulbecco's modified essential medium (DMEM, Cellgro) supplemented with 10% fetal bovine serum (FBS, HyClone) and 1% penicillin-streptomycin (Cellgro). The SKBR3 cell line was tested and free of mycoplasma contamination (MycoAlert Mycoplasma Detection Kit, Lonza, LT07-418).

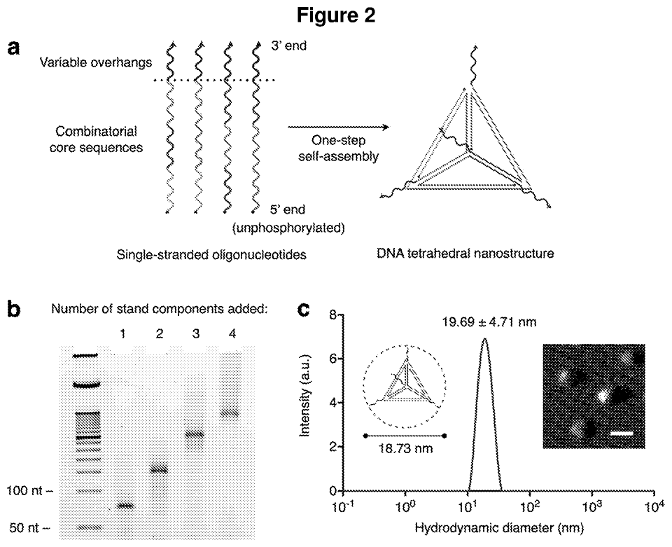

The nanostructure is assembled from four single-stranded DNA via a one-step self-assembly process (FIG. 2a). Each strand consists of a variable overhang (black) and combinatorial core sequences (regions of complementary subsequences, each of 20 bases in length, color-coded). When assembled, the overhangs extend from the four vertexes of the tetrahedron while the combinatorial sequences form the core of the nanostructure. DNA tetrahedron assembly and characterization. The four component DNA strands (Integrated DNA Technologies, IDT; Table 1) were mixed in TEM buffer (10 mM Tris, 1 mM EDTA, 5 mM MgCl2) to a final concentration of 1 μM each, heated to 95° C. for 2 min, then cooled to room temperature over 2 h. Native polyacrylamide gel electrophoresis was conducted to confirm the formation of the DNA tetrahedron. The complete stepwise formation of the DNA tetramer from its four constituent strands was monitored on a 6% PAGE gel (FIG. 2b). 18 μl of FAM-labeled DNA nanostructure (0.1 μM) was mixed with 2 μl of BlueJuice gel loading buffer (Invitrogen). The mixture was loaded to a 6% TBE gel (Invitrogen), run at 200 V for 20 min and imaged using a ChemiDoc Touch imaging system (Bio-Rad). The hydrodynamic diameter of the formed DNA nanostructures was measured using a Zetasizer Nano ZS instrument (Malvern). The nanostructure morphology was confirmed using atomic force microscopy. Briefly, DNA nanostructures (10 μl, diluted to 5 nM in TEM buffer) were dropped onto freshly cleaved mica and allowed to air dry. Once dried, 50 μl TEM buffer was added and the sample was scanned using an SNL-10 probe (Bruker) on a Bioscope Catalyst AFM (Bruker).

Antibody-DNA conjugation. Antibodies anti-HER2 antibody was activated by mixing with sulfo-SMCC (Pierce) at 50-fold molar excess in PBS pH 7.4 with 1 mM EDTA and incubated for 2 h at room temperature. The reaction was buffer exchanged with Zeba micro spin desalting columns (Pierce) to remove excess sulfo-SMCC. DNA strands (short and long) modified with thiol and 6-FAM (200 μM, IDT; Table 1) were activated by incubating with TCEP reducing gel (Pierce) to reduce the disulfide bonds for 1 h at room temperature. The reaction was then filtered and the gel washed several times to recover the activated DNA. The recovered DNA was concentrated using Amicon Ultra centrifugal filters (Millipore). The concentrations of the activated antibody and DNA were determined by absorbance measurements (Nanodrop, Thermo Fisher). The activated antibody was then mixed with excess activated DNA to a final concentration of 0.5 mg/ml and incubated overnight at 4° C. The reaction was filtered using Amicon Ultra centrifugal filters (100 kDa size cut-off, Millipore) and washed three times to remove unreacted DNA. The concentrations of the antibody-DNA conjugates were determined by BCA assay (Pierce) and the average numbers of DNA per antibody were estimated by fluorescence measurements (Spark 10M, Tecan). The hydrodynamic diameter and zeta potential of antibody and antibody-DNA conjugates were measured at 0.1 mg/ml using Zetasizer Nano ZS instrument (Malvern).

Evaluation of STAMP Performance.

To compare the performance of the STAMP assay with direct long DNA conjugates (Ab-long DNA), standard polystyrene beads with known binding capacity were used to ensure uniformity and enable validation through flow cytometry. HER2-modified beads were prepared by incubating streptavidin-coated 3.0 μm polystyrene beads (Spherotech) in 10 μg/ml biotinylated HER2 (Acro Biosystems) in PBS with 0.5% bovine serum albumin (BSA, Sigma) overnight at 4° C. The mixture was then centrifuged, washed, and resuspended in PBS with 0.5% BSA. The modified beads were subjected to the STAMP assay and targeting with the direct long DNA conjugates, before being analyzed with qPCR as previously described. All antibody binding was also cross-validated with flow cytometry. To assess the sensitivity of the STAMP assay for cell measurements, cell suspensions were prepared, counted using a Countess II automated cell counter (Invitrogen), before being serially diluted and subjected to the STAMP assay as previously described.

Flow Cytometry.

Cell suspensions were prepared and labeled with 5 μg/ml primary antibodies for 1 h at 4° C., as previously described. Following centrifugation and washing, cells were labeled with 2 μg/ml FITC-conjugated secondary antibody (Becton Dickinson) for 30 min at 4° C. and washed twice by centrifugation. FITC fluorescence was assessed using a LSRII flow cytometer (Becton Dickinson). Mean fluorescence intensity of all cells, excluding debris, was determined using FlowJo (version 10.4.2), and biomarker expression levels were normalized against isotype control antibodies.

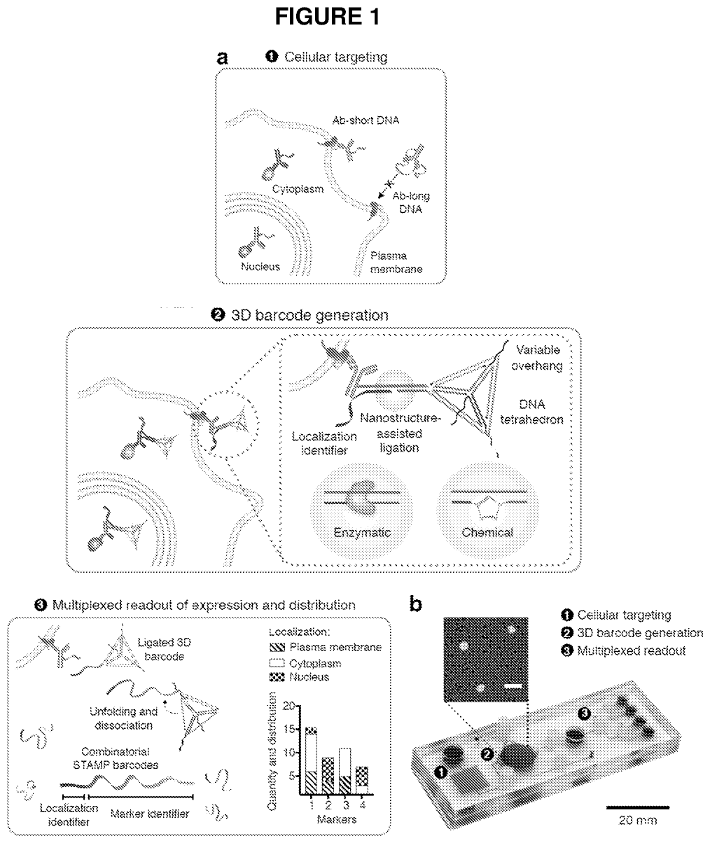

The STAMP platform is a 3D barcoding technology that comprises three functional steps: cellular targeting, 3D barcode generation, and multiplexed readout (FIG. 1a). In the targeting step, cells are incubated with a mixture of modified antibodies, each conjugated with a short DNA sequence (Ab-short DNA). In comparison to antibodies modified with long, PCR-compatible DNA strands (Ab-long DNA), which show disrupted target binding, the short conjugates retain their specificity to label proteins of various subcellular localizations (e.g., membrane, cytoplasm and nucleus). In the next step, 3D DNA barcodes are generated from the bound Ab-short DNA conjugates. We probe the antibodies with compact DNA assemblies (i.e., DNA tetrahedral nanostructures bearing combinatorial core sequences and variable overhangs, FIG. 2) as well as short localization identifiers, which are DNA labels differentially distributed across subcellular compartments. Simultaneous binding and enhanced ligation of both probes—achieved through structure-assisted enzymatic or chemical ligation—generate specific 3D barcodes in situ in whole cells. Upon heat inactivation, these structures further unfold and dissociate to liberate a pool of linear, combinatorial STAMP barcodes; each STAMP barcode reflects both the target marker's identity as well as its subcellular distribution pattern. High-throughput analysis of the STAMP barcodes, through established genomic platforms (e.g., qPCR and next generation sequencing), enables multiplexed profiling of protein expression and distribution in whole cells.

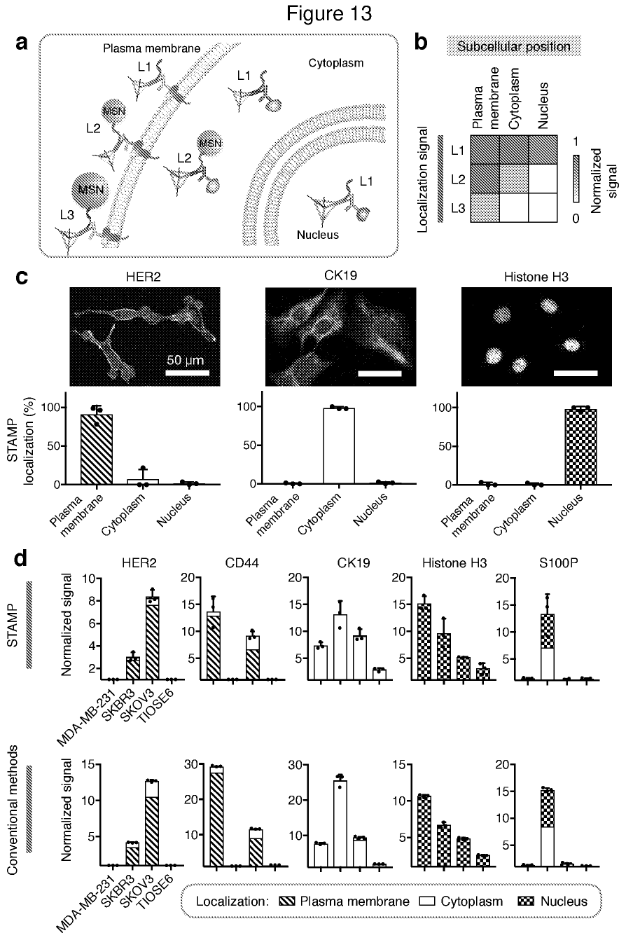

FIG. 1a depicts a schematic representation of the STAMP assay, not drawn to scale. The technology comprises three functional steps. For cellular targeting, antibodies conjugated with unique short DNA strands (Ab-short DNA) are used to label specific cellular proteins. In comparison to long DNA conjugates (Ab-long DNA), the Ab-short DNA conjugates can access and bind specifically to proteins of various subcellular localizations (e.g., membrane, cytoplasm and nucleus). Next, 3D barcodes are generated in situ from the bound antibodies, through nanostructure-assisted ligation of DNA tetrahedron probes with localization identifiers. The identifiers are short DNA labels which are differentially distributed across subcellular compartments and thus carry localization information (see FIG. 13a for details).

The nanostructure is assembled from four single-stranded DNA via a one-step self-assembly process (FIG. 2a). Each strand consists of a variable overhang (black) and combinatorial core sequences (regions of complementary sub-sequences, each of 20 bases in length). When assembled, the overhangs form the four vertexes of the tetrahedron while the combinatorial sequences form the core of the nanostructure. The complete stepwise formation of the DNA tetramer from its four constituent strands was monitored on a 6% PAGE gel (FIG. 2b). Dynamic light scattering measurement showed a monodispersed particle population with a mean diameter of ˜19.69 nm (FIG. 2c), which is in good agreement with the theoretical prediction (18.73 nm). Atomic force microscopy analysis (FIG. 2c inset) further confirmed the nanostructures' pyramidal morphology.

The DNA nanostructures can enhance the barcoding efficiency through both enzymatic as well as click chemical ligation. Once ligated, the 3D barcodes unfold and dissociate to liberate a pool of diverse, linear STAMP barcodes. Each STAMP barcode thus reflects the target marker's identity, quantity as well as its subcellular distribution pattern. High-throughput analysis of STAMP barcodes enables multiplexed measurements of the target markers' subcellular expression and distribution.

DNA tetrahedral nanostructures were prepared for 3D barcoding. The programmed organization (i.e., collective compact assembly and combinatorial subunit arrangement in constituent sequences) not only improved the barcoding kinetics, but also provided additional sequence information to facilitate the barcode differentiation, thereby generating STAMP barcodes of compatible length and variability for downstream genomic analysis. The nanostructures w3ere assembled from four DNA strands, each bearing combinatorial core sequences and a variable overhang, through a single-step annealing (FIG. 2a and Table 1). The complete assembly was monitored through native gel electrophoresis (FIG. 2b). The annealed structures demonstrated a unimodal hydrodynamic diameter ˜19.69 nm (FIG. 2c), which is in good agreement with their theoretical diameter (18.73 nm, of a tetrahedron with four overhangs at the vertexes). Atomic force microscopy (FIG. 2c, inset) further confirmed the pyramidal morphology of the nanostructures.

TABLE 1
Sequences for STAMP characterization
Characterization of antibody binding

| | | |
|---|---|---|
| SEQ ID NO. 1 | Short DNA | ACGGGTATGATACTTCTATGATCGTACGAT |
| | | (5′ Thiol Modifier C6 S-S, 3′ 6-FAM) |
| SEQ ID NO. 2 | Long DNA | ACGAACATTCCTAAGTCTGAAATTTATCACCCGCCAT |
| | | ATAGACGTATCACCAGGCAGTTGAGTTATCGTACGA |
| | | TCATAG |
| | | (5′ Thiol Modifier C6 S-S, 3′ 6-FAM) |

STAMP enzymatic ligation

| | | |
|---|---|---|
| SEQ ID NO. 3 | Tetrahedron 1 | ACGAACATTCCTAAGTCTGAAATTTATCACCCGCCAT |
| | | AGTAGACGTATCACCAGGCAGTTGAGTTATCGTACG |
| | | ATCATAG |
| SEQ ID NO. 4 | Tetrahedron 2 | ATTCAGACTTAGGAATGTTCGACATGCGAGGGTCCA |
| | | ATACCGACGATTACAGCTTGCTACACGITTACAGTC |
| | | GTATTGCA |
| SEQ ID NO. 5 | Tetrahedron 3 | ACTACTATGGCGGGTGATAAAACGTGTAGCAAGCTG |
| | | TAATCGACGGGAAGAGCATGCCCATCCTTATTCTAG |
| | | ACGTTACT |
| SEQ ID NO. 6 | Tetrahedron 4 | ACGGTATTGGACCCTCGCATGACTCAACTGCCTGGT |
| | | GATACGAGGATGGGCATGCTCTTCCCGTTTAACTAT |
| | | AGCTACAA |
| SEQ ID NO. 7 | Overhang | AAGTATCATACCCGTCTCCACGAAAAAA |
| | DNA | (5′ Phosphorylation) |

STAMP click ligation

| | | |
|---|---|---|
| SEQ ID NO. 8 | Tetrahedron 1 | ACGAACATTCCTAAGTCTGAAATTTATCACCCGCCAT |
| | Click | AGTAGACGTATCACCAGGCAGTTGAGTTATCGTACG |
| | | ATCATAG |
| | | (3′ Azide) |
| SEQ ID NO. 4 | Tetrahedron 2 | ATTCAGACTTAGGAATGTTCGACATGCGAGGGTCCA |
| | | ATACCGACGATTACAGCTTGCTACACGTTTACAGTC |
| | | GTATTGCA |
| SEQ ID NO. 5 | Tetrahedron 3 | ACTACTATGGCGGGTGATAAAACGTGTAGCAAGCTG |
| | | TAATCGACGGGAAGAGCATGCCCATCCTTATTCTAG |
| | | ACGTTACT |
| SEQ ID NO. 6 | Tetrahedron 4 | ACGGTATTGGACCCTCGCATGACTCAACTGCCTGGT |
| | | GATACGAGGATGGGCATGCTCTTCCCGTTTAACTAT |
| | | AGCTACAA |
| SEQ ID NO. 9 | Overhang | AAGTATCATACCCGTCTCCACGAAAAAA |
| | DNA Label | (5′ Hexynyl) |
| | Click | |

qPCR analysis primers

| | | |
|---|---|---|
| SEQ ID NO. 10 | Forward | ATCACCCGCCATAGTAGACG |
| | primer | |
| SEQ ID NO. 11 | Reverse | CGTGGAGACGGGTATGATACTT |
| | primer | |

qPCR analysis. For qPCR analysis of STAMP barcodes, 2 μl of sample from the STAMP assay was mixed with primers (300 nM) in PowerUp SYBR Green Master Mix (Applied Biosystems). qPCR analysis was performed on a QuantStudio 5 real-time PCR system (Applied Biosystems) under fast cycling protocol recommended by the manufacturer: 50° C. for 2 min, 95° C. for 2 min, 40 cycles of 95° C. for 1 s and 60° C. for 30 s. The signal (cycle number) obtained from each marker was normalized against that of IgG isotype control. We validated the amplification efficiency of all qPCR primer pairs in amplifying their respective targets (Table 3), through a serial dilution of DNA templates (0.1 μM to 0.1 fM). To confirm the specificity of the primer pairs for their respective targets, 10 μM of each DNA template was subjected to qPCR with all primer sets as described above.

### Example 2: System on a Miniaturized Microfluidic Platform

Microfluidic Device Fabrication.

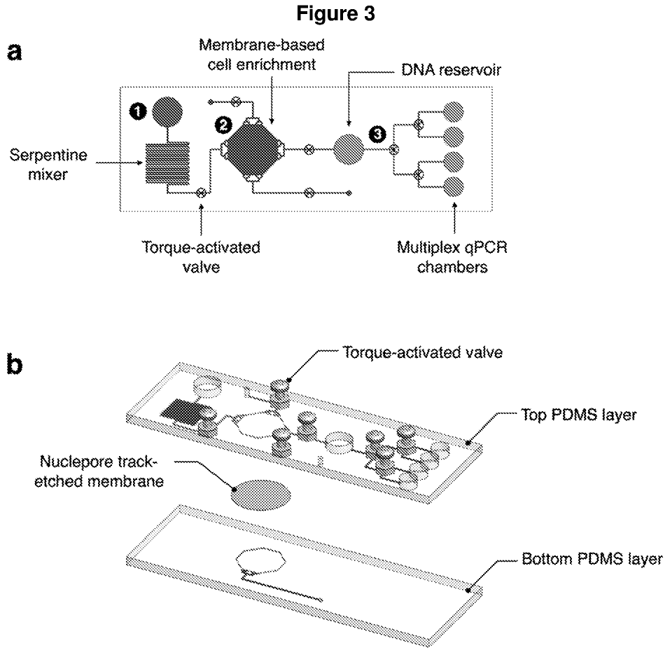

A prototype STAMP microfluidic device comprising 3 regions (FIG. 3) was fabricated from polydimethylsiloxane (PDMS, Dow Corning) and borosilicate glass. The fabrication of the microfluidic device involved plasma bonding a Nuclepore track-etched membrane (5-μm pore size, Whatman) between two layers of PDMS pieces (50 mTorr, 50 W, 1 min). The 50 μm-thick cast molds were prepared via conventional photolithography using SU-8 photoresist and silicon wafers. The PDMS replicas were made by pouring uncured PDMS (10:1 elastomer base to curing agent ratio) onto the cast molds. The polymer was cured at 75° C. for 30 min. The prepared PDMS layers were then assembled with the membrane. To prepare the torque-activated valves, a 100 μm-thick PDMS film was first spin-coated on the cast mold for the top PDMS piece. Subsequently, multiple nylon screws and hex nuts (RS Components) were positioned on the PDMS film over their respective channels and embedded in uncured PDMS, before a final curing step. We further lyophilized the qPCR primers in the multiplex chambers using a Freezone benchtop freeze dry system (Labconco). The device was flushed with ethanol and nuclease-free water before lyophilization.

The device consists of three compartments:
(1) a serpentine mixer for cell and antibody targeting,
(2) an embedded membrane (5-μm pore size) for cell enrichment and in situ STAMP barcode generation, and
(3) DNA reservoir and multiple chambers for amplification and multiplexed analysis of the generated barcodes.

Fluidic flow from one compartment to the next is controlled by torque activated valves (FIG. 3a). The microfluidic device is assembled from two polydimethylsiloxane (PDMS) layers to embed a porous membrane for cell enrichment and STAMP analysis (FIG. 3b).

STAMP barcode generation (microfluidic chip format).

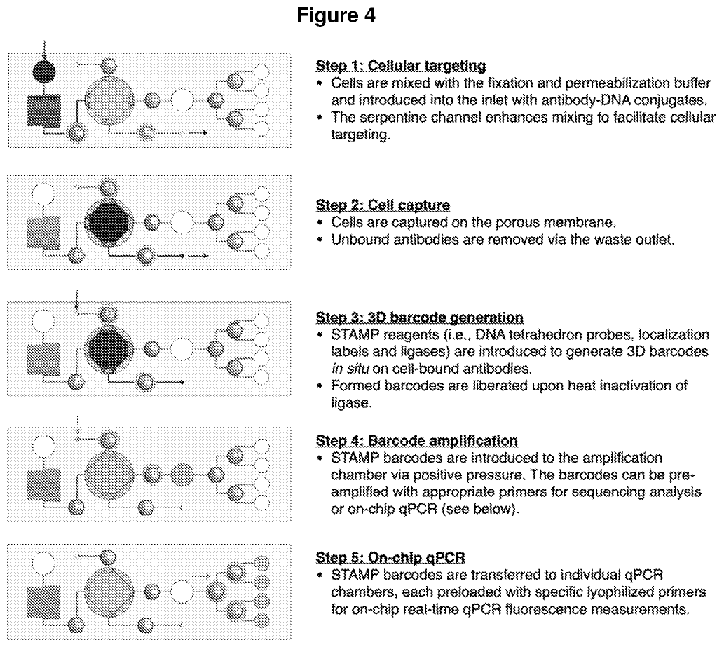

Operation steps of the microfluidic device are illustrated in FIG. 4. Firstly, 50 μl of crude sample, mixed in the fixation and permeabilization buffer, was loaded with the antibody-DNA conjugates into the cellular targeting chamber. Solution flow was actuated through the serpentine mixer at a flow rate of 5 μl/min via negative pressure (Harvard Instruments). Once the mixture entered the cell capture chamber, the targeted cells were trapped by the membrane filter (5 μm pore size, Nuclepore, Whatman), while the unbound antibodies were removed in the filtrate. 20 μl of STAMP assay mix comprising 0.5 μM DNA tetrahedron probes and localization labels, and 20 U/μl T4 DNA ligase in ligase buffer (New England Biolabs) was introduced into the cell capture chamber and incubated with the trapped cells for 10 min at room temperature for 3D barcoding. After barcode generation on the membrane (i.e., in situ hybridization and ligation), the formed barcodes were liberated upon heat inactivation of ligase (65° C., 10 min). We applied positive pressure at this point to transfer the unbound STAMP barcodes to the amplification chamber, while the cells remained bound on the membrane. In the open DNA reservoir, we mixed the barcode solution with PowerUp SYBR Green Master Mix (Applied Biosystems). The torque-activated valves were subsequently opened to allow assay distribution to the 4 multiplex qPCR chambers via capillary action, rehydrating the lyophilized qPCR primers. 2 μl of mineral oil (Sigma) was added to each reaction mixture to minimize evaporation during thermal cycling. qPCR could be performed on a custom-built flat-bed thermal cycler under standard cycling protocol. Real-time PCR fluorescence intensities were measured by a miniaturized fluorescence reader (ESElog, Qiagen). Clinical samples.

To facilitate clinical processing and multiplexed protein typing of rare cells, the STAMP technology was implemented on a miniaturized microfluidic platform (FIG. 1b). The STAMP microfluidic device of FIG. 1b is designed to complement the STAMP assay, particularly for processing of rare cells. It integrates a serpentine mixer 1 for efficient cellular targeting, an embedded, porous membrane 2 for rare cell enrichment and 3D barcode generation 3, and multiple chambers 4 for STAMP barcode amplification and multiplexed analysis.

The device was designed to complement the STAMP workflow (FIG. 3).

Specifically, it integrated three major functional components: i) a serpentine mixer 2 for efficient cellular targeting with antibodies, ii) an embedded, porous membrane 4 for rare cell enrichment and 3D barcoding of targeted cells (FIG. 1b, inset), and iii) multiple chambers 6 for STAMP barcode collection, amplification and analysis. The microfluidic platform could be loaded onto a custom-designed thermal cycling system for amplification of STAMP barcodes. All fluidic flow was controlled through torque-activated valves 8 to streamline the assay operation (FIG. 4).

### Example 3: Optimization of Modified Antibodies

Cell culture. All human cancer cell lines were obtained from American Type Culture Collection (ATCC). MDA-MB-231, SKBR3, SKOV3, cells were grown in Dulbecco's modified essential medium (DMEM, Cellgro) supplemented with 10% fetal bovine serum (FBS, HyClone) and 1% penicillin-streptomycin (Cellgro). All cell lines were tested and free of mycoplasma contamination (MycoAlert Mycoplasma Detection Kit, Lonza, LT07-418).

DNA tetrahedron assembly and characterization. The four component DNA strands (Integrated DNA Technologies, IDT; Table 1) were mixed in TEM buffer (10 mM Tris, 1 mM EDTA, 5 mM MgCl2) to a final concentration of 1 μM each, heated to 95° C. for 2 min, then cooled to room temperature over 2 h. Native polyacrylamide gel electrophoresis was conducted to confirm the formation of the DNA tetrahedron. 18 μl of FAM-labeled DNA nanostructure (0.1 μM) was mixed with 2 μl of BlueJuice gel loading buffer (Invitrogen). The mixture was loaded to a 6% TBE gel (Invitrogen), run at 200 V for 20 min and imaged using a ChemiDoc Touch imaging system (Bio-Rad). The hydrodynamic diameter of the formed DNA nanostructures was measured using a Zetasizer Nano ZS instrument (Malvern). The nanostructure morphology was confirmed using atomic force microscopy. Briefly, DNA nanostructures (10 μl, diluted to 5 nM in TEM buffer) were dropped onto freshly cleaved mica and allowed to air dry. Once dried, 50 μl TEM buffer was added and the sample was scanned using an SNL-10 probe (Bruker) on a Bioscope Catalyst AFM (Bruker).

Antibody-DNA conjugation. Antibodies anti-HER2 and anti-EGFR were activated by mixing with sulfo-SMCC (Pierce) at 50-fold molar excess in PBS pH 7.4 with 1 mM EDTA and incubated for 2 h at room temperature. The reaction was buffer exchanged with Zeba micro spin desalting columns (Pierce) to remove excess sulfo-SMCC. DNA strands (short and long) modified with thiol and 6-FAM (200 μM, IDT; Table 1) were activated by incubating with TCEP reducing gel (Pierce) to reduce the disulfide bonds for 1 h at room temperature. The reaction was then filtered and the gel washed several times to recover the activated DNA. The recovered DNA was concentrated using Amicon Ultra centrifugal filters (Millipore). The concentrations of the activated antibody and DNA were determined by absorbance measurements (Nanodrop, Thermo Fisher). The activated antibody was then mixed with excess activated DNA to a final concentration of 0.5 mg/ml and incubated overnight at 4° C. The reaction was filtered using Amicon Ultra centrifugal filters (100 kDa size cut-off, Millipore) and washed three times to remove unreacted DNA. The concentrations of the antibody-DNA conjugates were determined by BCA assay (Pierce) and the average numbers of DNA per antibody were estimated by fluorescence measurements (Spark 10M, Tecan). The hydrodynamic diameter and zeta potential of antibody and antibody-DNA conjugates were measured at 0.1 mg/ml using Zetasizer Nano ZS instrument (Malvern).

qPCR analysis. For qPCR analysis of STAMP barcodes, 2 μl of sample from the STAMP assay was mixed with primers (300 nM) in PowerUp SYBR Green Master Mix (Applied Biosystems). qPCR analysis was performed on a QuantStudio 5 real-time PCR system (Applied Biosystems) under fast cycling protocol recommended by the manufacturer: 50° C. for 2 min, 95° C. for 2 min, 40 cycles of 95° C. for 1 s and 60° C. for 30 s. The signal (cycle number) obtained from each marker was normalized against that of IgG isotype control. We validated the amplification efficiency of all qPCR primer pairs in amplifying their respective targets (Table 3), through a serial dilution of DNA templates (0.1 μM to 0.1 fM). To confirm the specificity of the primer pairs for their respective targets, 10 μM of each DNA template was subjected to qPCR with all primer sets as described above.

Evaluation of STAMP Performance.

To compare the performance of the STAMP assay with direct long DNA conjugates (Ab-long DNA), standard polystyrene beads with known binding capacity were used to ensure uniformity and enable validation through flow cytometry. HER2-modified beads were prepared by incubating streptavidin-coated 3.0 μm polystyrene beads (Spherotech) in 10 μg/ml biotinylated HER2 (Acro Biosystems) in PBS with 0.5% bovine serum albumin (BSA, Sigma) overnight at 4° C. The mixture was then centrifuged, washed, and resuspended in PBS with 0.5% BSA. The modified beads were subjected to the STAMP assay and targeting with the direct long DNA conjugates, before being analyzed with qPCR as previously described. All antibody binding was also cross-validated with flow cytometry. To assess the sensitivity of the STAMP assay for cell measurements, cell suspensions were prepared, counted using a Countess II automated cell counter (Invitrogen), before being serially diluted and subjected to the STAMP assay as previously described.

Flow Cytometry.

Cell suspensions were prepared and labeled with 5 μg/ml primary antibodies for 1 h at 4° C., as previously described. Following centrifugation and washing, cells were labeled with 2 μg/ml FITC-conjugated secondary antibody (Becton Dickinson) for 30 min at 4° C. and washed twice by centrifugation. FITC fluorescence was assessed using a LSRII flow cytometer (Becton Dickinson). Mean fluorescence intensity of all cells, excluding debris, was determined using FlowJo (version 10.4.2), and biomarker expression levels were normalized against isotype control antibodies.

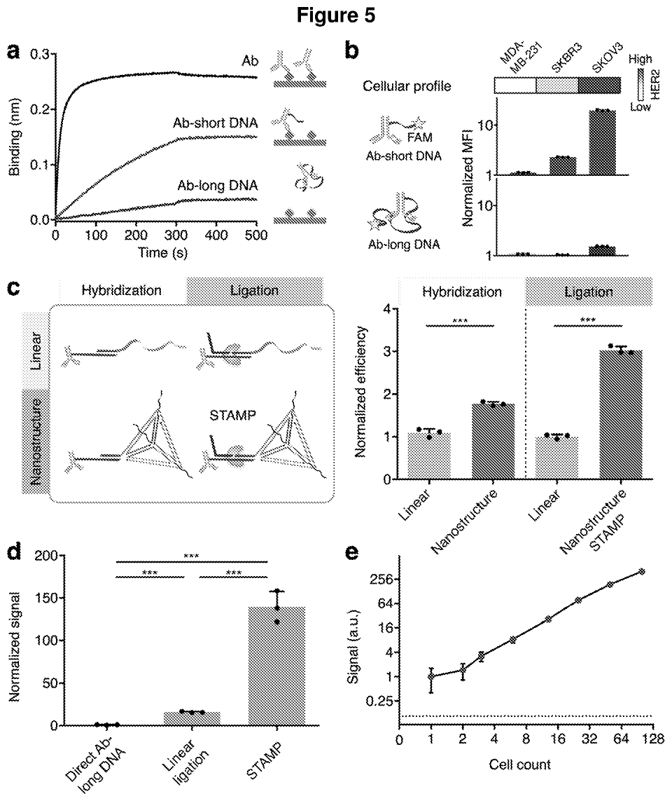

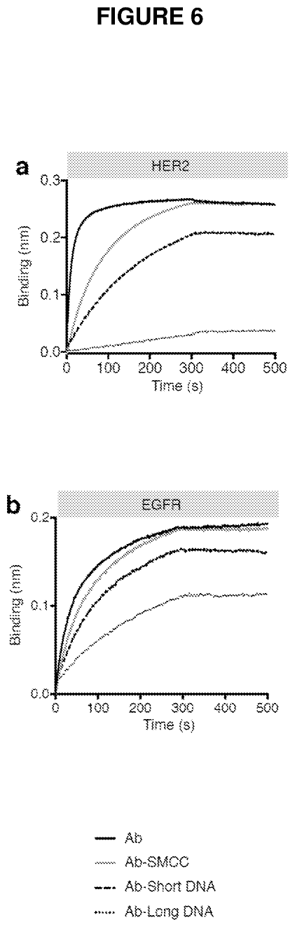

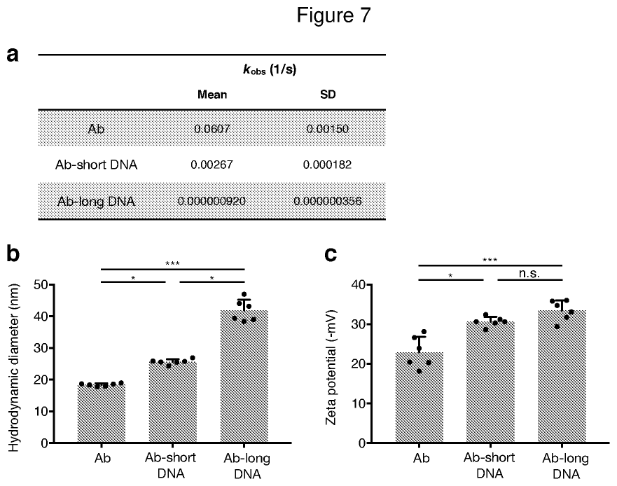

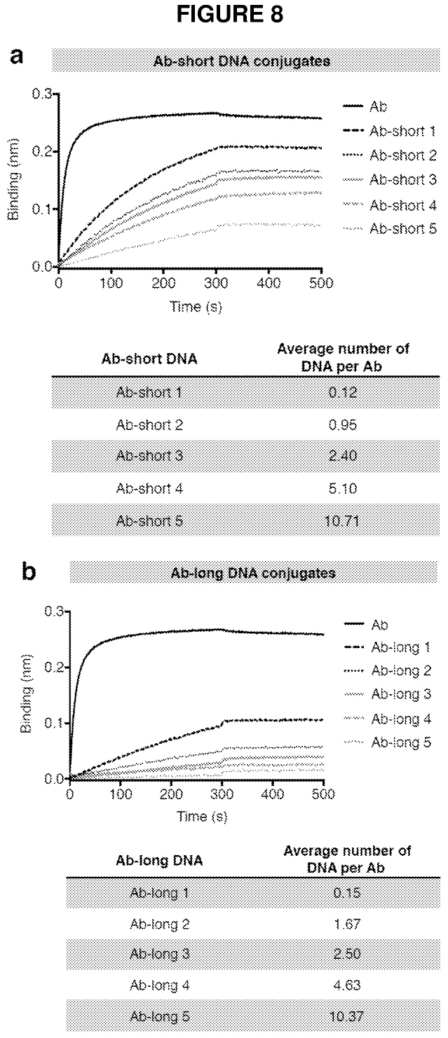

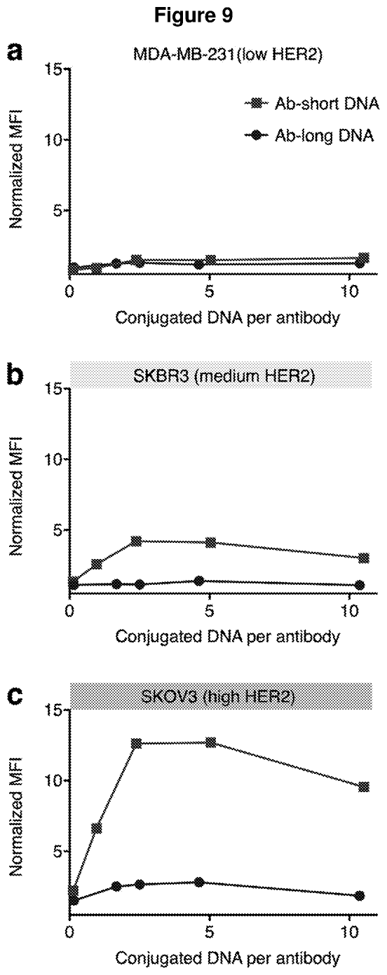

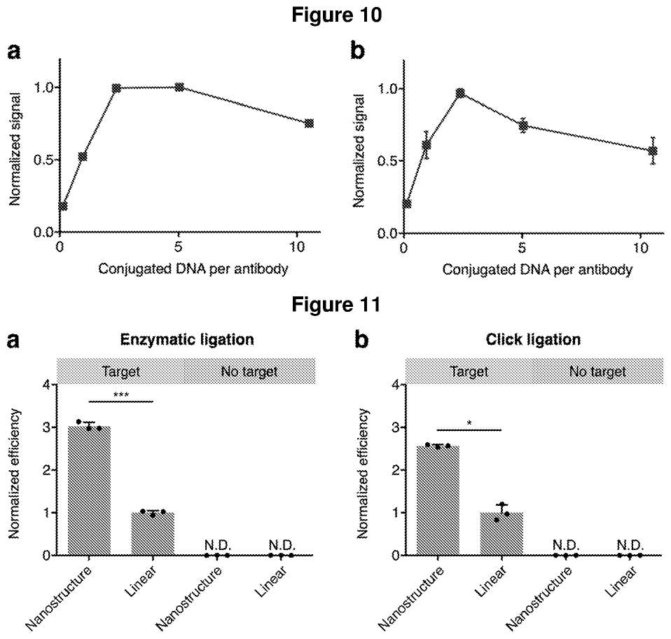

In developing the STAMP platform, the 3D barcoding efficiency was first evaluated. To optimize the effects of DNA modification on antibody targeting, antibodies (anti-HER2, anti-EGFR) were conjugated with DNA strands of different lengths, short (30 bases) and long (80 bases—a typical barcode length compatible with direct PCR analysis) (Table 1). All conjugations were performed through standard NHS-maleimide coupling. Using optical interferometry sensors, we monitored the antibody binding kinetics to target proteins in real time (FIG. 5a and FIG. 6). In these tested antibodies, the long DNA conjugates showed the most significant decrease in antibody performance. While the long DNA conjugate showed little binding (kobs ˜66,000-fold lower than that of the native antibody), the short DNA conjugate could preserve its binding affinity (FIG. 7a). We attribute this performance difference to DNA-induced biophysical changes and steric hindrance on the antibody (FIG. 7b-c); these effects could thus be mitigated by regulating the DNA sequence length and conjugation density (FIG. 8). Using cell line models that express different levels of target proteins, these findings were further validated and optimized the DNA modification length and density (FIGS. 9-10). It was determined that an optimal conjugation ratio of about 2.5 short DNA strands per antibody could not only preserve the antibody affinity but also accurately reflect the cellular expression trend (FIG. 5b).

Using optical biolayer interferometry, the real-time binding of native antibodies (Ab), was monitored. SMCC crosslinker-conjugated antibodies (Ab-SMCC) and DNA-conjugated antibodies (Ab-short DNA, 30-base; Ab-long DNA, 80-base suitable for direct PCR amplification) with immobilized protein antigens for HER2 (FIG. 6a) and EGFR (FIG. 6b).

Anti-HER2 antibodies were modified with short (30-base) and long (80-base) DNA strands, respectively. Both modified antibodies bear on average ˜2.5 DNA strands/antibody. kobs values were determined based on the antibodies' binding kinetics to the target protein (HER2 receptor). The Ab-long DNA conjugate showed a drastic decrease in association kinetics, in comparison to the native antibody and the Ab-short DNA conjugate (FIG. 7a). Changes in hydrodynamic diameter (FIG. 7b) and zeta potential of the antibody-DNA conjugates, as determined by dynamic light scattering analysis (FIG. 7c). The Ab-long DNA conjugate showed the most significant increase in hydrodynamic size and surface negative charge. Antibody-DNA conjugates are more negatively charged as compared to native antibody due to the DNA phosphate groups.

Real time binding kinetics were measures (FIG. 8). The associated tables (FIGS. 8a and 8b below) summarize the average number of DNA strands per antibody, as measured by spectroscopic measurements. For both groups of antibody conjugates, an increase in DNA conjugation density reduced the antibody affinity. When comparing between conjugates of comparable number of DNA strands, the short DNA conjugates (Ab-short DNA) could preserve more binding affinity, while the long DNA conjugates (Ab-long DNA) showed a more significant loss of antibody affinity. As more DNA strands were added to the antibody, the affinity of the Ab-long DNA conjugates reduced more significantly in comparison to that of the Ab-short DNA conjugates.

Targeted cells were analyzed by flow cytometry. The short DNA conjugates (Ab-short DNA) not only generated higher binding signals as compared to the long DNA conjugates (Ab-long DNA), but could also reflect the relative protein expression trend of the chosen cell lines (FIG. 9). With its high signal-to-noise ratio, a conjugation level of ˜2.5 short DNA strands per antibody was determined to be optimal for STAMP assay development.

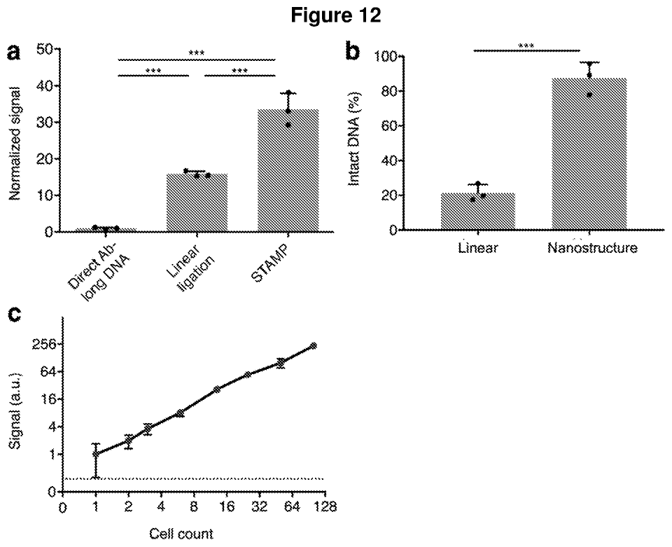

With the optimized Ab-short DNA conjugate, we next evaluated the efficiency of nanostructure assisted reactions in generating STAMP barcodes. For all comparative studies of hybridization and ligation, we used DNA tetrahedral nanostructures (assembled from four 80-base DNA strands, FIG. 2a) and the corresponding linear DNA strands (80-base, as a control); both bear an identical 15-base overhang complementary to the antibody-DNA conjugate (FIG. 5c, left; Table 1). As compared to the linear control probe, the nanostructure improved not only the DNA hybridization but also the ligation efficiencies (FIG. 5c, right). Importantly, this structure-assisted enhancement could be observed across different DNA ligation strategies (i.e., enzymatic ligation and click chemical ligation) (FIG. 11). We attribute this enhancement to improved DNA hybridization and enzyme recruitment to the DNA nanostructures; the presence of DNA nanostructures which consist of high-density, double-stranded DNA in close proximity to the ligation site could facilitate ligase recruitment, thereby improving the enzyme's local concentration to enhance ligation efficiency. All STAMP barcodes generated were amplified and quantified with standard qPCR analysis. In comparison to the directly-conjugated long DNA, STAMP with short DNA showed a significant signal enhancement (FIG. 12a) and demonstrated >100-fold improvement in signal amplification even in the presence of degrading serum nucleases (FIG. 5d). This boost in analytical signal could be attributed to STAMP's optimized antibody-DNA targeting, structure-enhanced reactions, as well as improved stability against DNA degradation (FIG. 12b). Importantly, when applied to cellular profiling (e.g., plasma membrane and intracellular markers), STAMP barcodes could be generated in situ from whole cells to achieve single-cell detection sensitivity (FIG. 5e and FIG. 12c). Using published data on the numbers of protein receptors per cell, we further estimated STAMP's sensitivity to a limit of detection in the range of 10-22 mol protein copies.

In comparison to immunolabeling with direct conjugate (Ab-long DNA), the STAMP assay demonstrated >30-fold signal improvement in PBS (FIG. 12a). Signals were determined by qPCR analysis and normalized against that of equivalently modified IgG isotype control antibodies. DNA tetrahedron (nanostructure) and single-stranded linear DNA (linear) were incubated in 70% serum for 1 h. The percentage of intact DNA was measured by qPCR. The nanostructure showed significantly higher stability than the linear DNA (FIG. 12b). A known concentration of breast cancer cells (SKBR3) was serially diluted, cell number validated, and STAMP measurements were performed for the cytoplasmic protein marker CK19. STAMP analysis showed single-cell level detection sensitivity (FIG. 12c).

In both ligation experiments, the ligation efficiencies of the DNA tetrahedral probe (Nanostructure) were compared against that of a comparable linear DNA probe (Linear), in the presence of a complementary target strand (Target) (FIG. 11). All experiments included appropriate negative controls, where the probes were ligated without the complementary target strand (No target). Ligation efficiency was determined from qPCR analysis of specific ligated products. The nanostructure probe showed enhanced ligation efficiency as compared to the linear probe.

Using optical biolayer interferometry, the realtime binding of native antibodies (anti-HER2) and conjugated antibodies (Ab-short DNA, 30-base; Ab-long DNA, 80-base suitable for direct PCR amplification) was monitored with immobilized protein antigens (HER2 receptor). Both modified antibodies were conjugated with a comparable number of DNA strands. While the Ab-long DNA showed little binding, the Ab-short DNA retained significantly higher binding affinity. All antibodies were labeled with an equivalent amount of fluorescence (FAM) and used to measure HER2 protein expression of various human cell lines (FIG. 5b). As compared to the Ab-long DNA conjugate, the Ab-short DNA conjugate showed better cellular targeting, and accurately reflected the protein expression trend. All signals were normalized against that of equivalently modified IgG isotype control antibodies. Comparison of linear and nanostructure DNA probes (FIG. 5c Left). With the optimized Ab-short DNA conjugate, the hybridization and ligation efficiencies of long linear DNA probe (80-base) and tetrahedral nanostructure probe (composed of four 80-base DNA strands), respectively were compared. All hybridization and enzymatic ligation products were measured through qPCR analysis (FIG. 5c Right). In comparison to the long linear DNA probe, the nanostructure probe not only showed an improvement in hybridization efficiency, but also demonstrated enhanced ligation efficiency for the STAMP assay. The nanostructure-enhanced ligation efficiency could also be observed with click chemical ligation (see FIG. 11 for details). In comparison to direct labeling and detection with the Ab-long DNA conjugate, the STAMP assay demonstrated >100-fold signal improvement (FIG. 5d). All assays were performed in the presence of degrading serum nucleases. Signals were determined by qPCR analysis and normalized against that of equivalently modified IgG isotype control antibodies. Human breast cancer cells (SKBR3) were serially diluted and subjected to STAMP measurements of HER2 (FIG. 5e). Dotted line shows the limit of detection, defined as 3× s.d. of no-cell control. All measurements were performed in triplicates, and the data in FIG. 5b-e are presented as mean±s.d. (***P<0.0005, Student's t-test). MFI, mean fluorescence intensity arbitrary unit (a.u.).

### Example 4: Subcellular Protein Quantification and Distribution

Cell culture. All human cancer cell lines were obtained from American Type Culture Collection (ATCC). MDA-MB-231, SKBR3, SKOV3, CaOV3, OVCAR3, MCF7, and BT474 were grown in Dulbecco's modified essential medium (DMEM, Cellgro) supplemented with 10% fetal bovine serum (FBS, HyClone) and 1% penicillin-streptomycin (Cellgro). OV90, OVCA429, and UCI101 were cultured in RPMI-1640 medium (Cellgro) supplemented with 10% FBS and 1% penicillin streptomycin. HME1 (ATCC) was cultured in mammary epithelial cell growth medium (MEGM) BulletKit (Lonza). Normal ovarian surface epithelium (NOSE) cell lines were derived from ovarian surface epithelium (OSE) brushings and cultured in 1:1 mixture of MCDB 105 medium and Medium 199 (Sigma-Aldrich) with gentamicin (25 μg/ml) and 15% heat-inactivated serum. TIOSE4 and TIOSE6 cell lines were then obtained by transfecting NOSE cells with hTERT, and cultured in RPMI-1640 supplemented with 10% FBS and 1% penicillin-streptomycin. All cell lines were tested and free of mycoplasma contamination (MycoAlert Mycoplasma Detection Kit, Lonza, LT07-418).

DNA tetrahedron assembly and characterization. The four component DNA strands (Integrated DNA Technologies, IDT; Table 1) were mixed in TEM buffer (10 mM Tris, 1 mM EDTA, 5 mM MgCl2) to a final concentration of 1 μM each, heated to 95° C. for 2 min, then cooled to room temperature over 2 h. Native polyacrylamide gel electrophoresis was conducted to confirm the formation of the DNA tetrahedron. 18 μl of FAM-labeled DNA nanostructure (0.1 μM) was mixed with 2 μl of BlueJuice gel loading buffer (Invitrogen). The mixture was loaded to a 6% TBE gel (Invitrogen), run at 200 V for 20 min and imaged using a ChemiDoc Touch imaging system (Bio-Rad). The hydrodynamic diameter of the formed DNA nanostructures was measured using a Zetasizer Nano ZS instrument (Malvern). The nanostructure morphology was confirmed using atomic force microscopy. Briefly, DNA nanostructures (10 μl, diluted to 5 nM in TEM buffer) were dropped onto freshly cleaved mica and allowed to air dry. Once dried, 50 μl TEM buffer was added and the sample was scanned using an SNL-10 probe (Bruker) on a Bioscope Catalyst AFM (Bruker).

Antibody-DNA conjugation. Antibodies (Table 2) were activated by mixing with sulfo-SMCC (Pierce) at 50-fold molar excess in PBS pH 7.4 with 1 mM EDTA and incubated for 2 h at room temperature. The reaction was buffer exchanged with Zeba micro spin desalting columns (Pierce) to remove excess sulfo-SMCC. DNA strands (short and long) modified with thiol and 6-FAM (200 μM, IDT; Supplementary Table 1) were activated by incubating with TCEP reducing gel (Pierce) to reduce the disulfide bonds for 1 h at room temperature. The reaction was then filtered and the gel washed several times to recover the activated DNA. The recovered DNA was concentrated using Amicon Ultra centrifugal filters (Millipore). The concentrations of the activated antibody and DNA were determined by absorbance measurements (Nanodrop, Thermo Fisher). The activated antibody was then mixed with excess activated DNA to a final concentration of 0.5 mg/ml and incubated overnight at 4° C. The reaction was filtered using Amicon Ultra centrifugal filters (100 kDa size cut-off, Millipore) and washed three times to remove unreacted DNA. The concentrations of the antibody-DNA conjugates were determined by BCA assay (Pierce) and the average numbers of DNA per antibody were estimated by fluorescence measurements (Spark 10M, Tecan). The hydrodynamic diameter and zeta potential of antibody and antibody-DNA conjugates were measured at 0.1 mg/ml using Zetasizer Nano ZS instrument (Malvern).

qPCR analysis. For qPCR analysis of STAMP barcodes, 2 μl of sample from the STAMP assay was mixed with primers (300 nM) in PowerUp SYBR Green Master Mix (Applied Biosystems). qPCR analysis was performed on a QuantStudio 5 real-time PCR system (Applied Biosystems) under fast cycling protocol recommended by the manufacturer: 50° C. for 2 min, 95° C. for 2 min, 40 cycles of 95° C. for 1 s and 60° C. for 30 s. The signal (cycle number) obtained from each marker was normalized against that of IgG isotype control. We validated the amplification efficiency of all qPCR primer pairs in amplifying their respective targets (Table 3), through a serial dilution of DNA templates (0.1 μM to 0.1 fM). To confirm the specificity of the primer pairs for their respective targets, 10 μM of each DNA template was subjected to qPCR with all primer sets as described above.

Localization identifiers: Mesoporous silica nanoparticle (MSN) preparation and application.

To prepare the small MSNs, 2 g of cetyltrimethylammonium bromide (CTAB, Sigma-Aldrich) and 40 μl of triethanolamine (TEA, Sigma-Aldrich) were dissolved in 20 mL of water and heated to 90° C. 1.5 ml of tetraethyl orthosilicate (TEOS, Sigma-Aldrich) was then added and allowed to react for 15 min, followed by addition of 300 μl (3-aminopropyl)triethoxysilane (APTES, Sigma-Aldrich). The solution was allowed to react for 1 h. To prepare the large MSNs, 50 mg of CTAB and 14 mg of sodium hydroxide were dissolved in 25 mL of water and heated to 80° C., followed by addition of 250 μl TEOS. After 1 h, 50 μl of TEOS and 50 μl of APTES were added to the mixture and allowed to react for another hour. The formed MSNs were washed with ethanol and water, refluxed in methanol overnight, and finally resuspended in PBS. Transmission electron microscopy (TEM) images of the MSNs were obtained using a JEM-2010F TEM (JEOL).

Immunofluorescence.

To measure subcellular protein localization and distribution, cells were cultured on a 8-well chamber slide (Nunc). Cells were fixed and permeabilized, as previously described, and blocked with 5% BSA for 1 h at room temperature. The prepared cells were labelled with 10 μg/ml of primary antibodies overnight at 4° C., washed twice with PBS, and then incubated with 2 μg/ml secondary antibodies for 1 h at room temperature. All cells were also stained with nuclear dye Hoechst 33342 (Molecular Probes) for 5 min at room temperature, before being mounted (Vector Laboratories). Fluorescence images were acquired using a Leica TCS SP8 confocal microscope (Leica Microsystems) at 20× magnification.

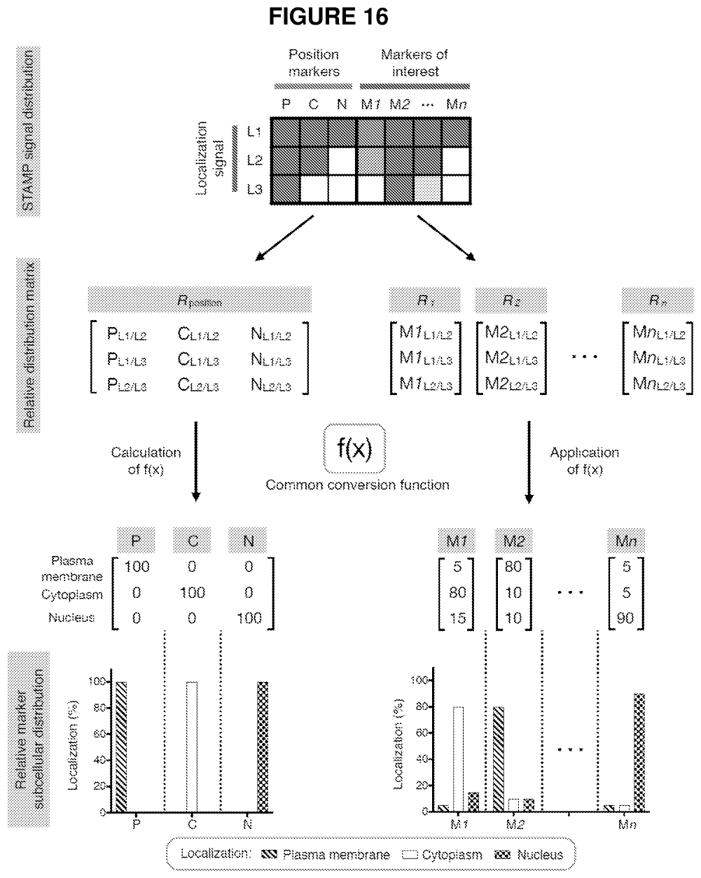

To conjugate the MSNs with DNA localization labels, the particles were activated with 2 mM sulfo-SMCC (Pierce), and washed to remove excess reagents. Localization DNA labels, modified with thiol (200 μM, IDT; Table 3), were activated as previously described, and added to the MSNs. The reaction was incubated overnight at 4° C., and filtered using Amicon Ultra centrifugal filters (Millipore) to remove unbound DNA. To investigate marker subcellular distribution, STAMP was performed as previously described, by incubating targeted cells with a mixture of DNA nanostructures and MSN-conjugated localization labels simultaneously. STAMP barcodes containing localization information were measured and data analysis (see FIG. 16 for details) was performed using the R-package (version 3.5.0).

To determine the subcellular distribution of individual markers of interest (M₁-Mₙ), in each experiment and analysis, we include three position markers as intrinsic spatial references, for plasma membrane (P), cytoplasm (C) and nucleus (N), respectively (see FIG. 16 for details). For all markers, the STAMP assay generates a signal distribution map, which comprises information of both marker abundance as well as subcellular localization. To determine the markers' relative subcellular distribution, regardless of their absolute abundance, we arrange the STAMP distribution map as a relative distribution matrix (R). For the position markers, Rₚₒₛᵢₜᵢₒₙ is a 3×3 matrix, with ratios of the different localization signals as the matrix elements. Specifically, for each position marker, its three ratios of localization signals (i.e., L1/L2, L1/L3 and L2/L3) collectively reflect the marker's subcellular distribution. As the position markers were chosen for their established and predominant localization, we assume that these markers completely reside in one location. We thus use Rₚₒₛᵢₜᵢₒₙ to solve for a conversion function, f(x), to reflect this protein distribution. Applying the generated conversion function, we process the STAMP signals of the markers of interest to determine their relative subcellular distribution.

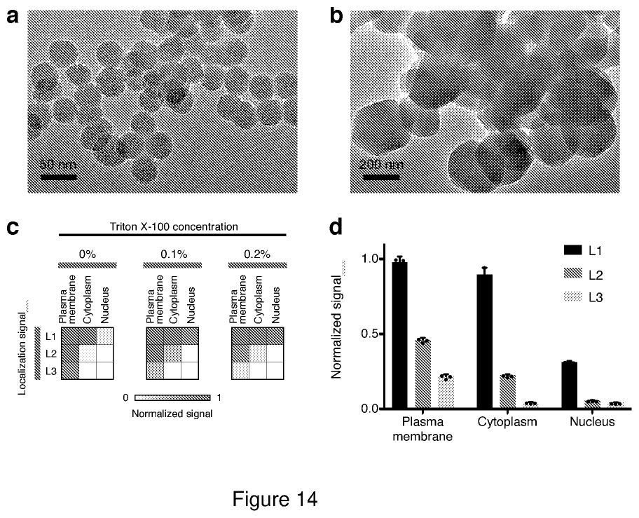

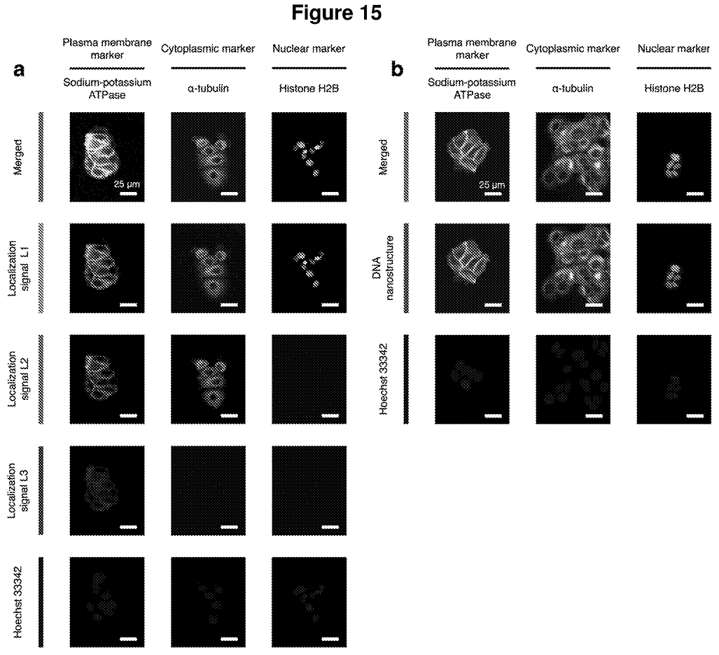

Leveraging the enhanced 3D barcoding the STAMP technology, was further extended to measure proteins of various subcellular localizations and determine their distributions within cells. Specifically, to form STAMP barcodes with localization information, the protein identifying DNA nanostructures were coupled with localization identifiers (i.e., L1, L2 and L3) (FIG. 13a). The short localization sequences were respectively conjugated to no (L1), small (L2) or large (L3) mesoporous silica nanoparticles (MSNs). In SKOV3 cells pre-targeted with antibody-DNA conjugates against known position markers (i.e., plasma membrane/sodium-potassium ATPase, cytoplasm/a-tubulin, and nucleus/histone H2B), the localization labels were incubated and their relative binding to these position markers measured in different subcellular compartments. All fluorescence measurements were normalized against respective marker expressions (column-wise), to account for differences in the expression levels of position markers. The data were globally normalized and presented as a heat map. By controlling the size of the silica nanoparticles (FIG. 14a-b) as well as cellular permeability (FIG. 14c), the localization labels could be differentially distributed across subcellular compartments (i.e., plasma membrane, cytoplasm and nucleus) (FIG. 13b and FIG. 14d). To form multiplexed STAMP barcodes, the DNA nanostructures and the localization labels were added simultaneously to cells for in situ 3D barcoding. For all measurements, three known position markers were included as intrinsic spatial references, namely sodium-potassium ATPase (plasma membrane), a-tubulin (cytoplasm), and histone H2B (nucleus) (FIG. 15 and Table 2). These position markers were chosen for their established and predominant localization to a single subcellular compartment. Using these position markers' relative STAMP distribution patterns, we could solve for a conversion function to transform all STAMP measurements and determine the subcellular distributions of markers of interest (FIG. 16). This data analysis thus takes into account the signal differences from the localization labels, and is designed with intrinsic spatial references and matrix conversions to correct for these signal differences.

TABLE 2
Protein markers and antibodies used.

| | | Source of |
|---|---|---|
| Protein markers | Descriptions | Antibodies used |
| Sodium- | An enzyme found in the plasma membrane of all | Abcam, clone |
| potassium | animal cells, actively pumps sodium out of cells | EP1845Y |
| ATPase | while pumping potassium into cells | |
| α-tubulin | Together with beta-tubulin, polymerized into | Invitrogen, clone |
| | microtubules, a major component of the | 236-10501 |
| | eukaryotic cytoskeleton. | |
| Histone H2B | One of histone proteins that package and order | Invitrogen, clone |
| | the DNA into nucleosomes. Found in all | 18HCLC |
| | eukaryotic cells. | |
| HER2 | Human epidermal growth factor receptor 2, also | Roche, |
| | known as receptor tyrosine kinase erbB-2, | Trastuzumab |
| | whose overexpression plays a major role in the | |
| | development and progression of multiple | |
| | cancers. | |
| CK19 | Cytokeratin 19, the most used marker for the | Invitrogen, clone |
| | detection of tumor cells disseminated in the | RCK108 |
| | lymph nodes, peripheral blood, and bone | |
| | marrow of breast cancer patients. | |
| Histone H3 | One of histone proteins that package and order | Invitrogen, catalog |
| | the DNA into nucleosomes. Found in all | #PA5-11186 |
| | eukaryotic cells. | |
| CD44 | A cell surface glycoprotein involved in cell-cell | BD Biosciences, |
| | interactions, cell adhesion and migration, | clone 515 |
| | reported as markers for some breast and | |
| | prostate cancer stem cells. | |
| S100P | Calcium-binding protein P, localized in the | R&D Systems, |
| | cytoplasm and/or nucleus, involved in the | clone 357517 |
| | regulation of various cellular processes. | |
| EpCAM | Epithelial cell adhesion molecule, | R&D Systems, |
| | transmembrane glycoprotein expressed | clone 158206 |
| | exclusively in epithelial and epithelial | |
| | neoplasms. | |
| CA125 | Cancer antigen 125, also known as mucin 16 | Abcam, clone X75 |
| | (MUC16), the most frequently used biomarker | |
| | for ovarian cancer. | |
| CD24 | A small heavily glycosylated cell adhesion | eBioscience, clone |
| | molecule, expressed in hematological | eBioSN3 |
| | malignancies and solid tumors. | |
| TSPAN8 | Tetraspanin-8, cell surface glycoprotein | BioLegend, clone |
| | expressed in different carcinomas, used to | TAL69 |
| | predict overall survival of breast cancer patients. | |
| ER | Estrogen receptor, intracellular receptors | R&D Systems, |
| | activated by the hormone estrogen, | clone H4624 |
| | overexpressed in around 70% of breast cancer | |
| | cases | |
| PR | Progesterone receptor, intracellular receptors | Invitrogen, clone |
| | activated by the hormone progesterone, | alpha PR-22 |
| | involved in breast cancer development. | |
| CD9 | A transmembrane glycoprotein in the tetraspanin | BD Biosciences, |
| | family, expressed on platelets, pre-B cells, | clone M-L13 |
| | monocytes, endothelial and epithelial cells. | |
| VEGFR | Vascular endothelial growth receptor, a tyrosine | R&D Systems, |
| | kinase receptor, involved in vasculogenesis and | clone 49560 |
| | angiogenesis. | |
| EGFR | Epidermal growth factor receptor, a cell-surface | Merck, Cetuximab |
| | receptor whose overexpression and mutations | |
| | have been associated with many cancers. | |
| CD45 | Encoded by the PTPRC gene, a type I | BioLegend, clone |
| | transmembrane protein expressed on all | HI30 |
| | leukocytes. | |
| CD41 | Also known as integrin alpha chain 2b, a | BioLegend, clone |
| | heterodimeric integral membrane protein | HIP8 |
| | expressed on platelets | |

The short DNA localization labels (i.e., L1, L2 and L3) were attached to their respective carriers of varying sizes (i.e., none, small and large MSNs). Fluorescent DNA sequences were used for subcellular distribution analysis. By permeabilizing SKOV3 cells with different amounts of Triton X-100, the concentrations of these localization signals were measured in different subcellular compartments, through known position markers (plasma membrane/sodium-potassium ATPase, cytoplasm/a-tubulin, and nucleus/histone 1H2B). All fluorescence measurements were normalized against respective marker expressions. 0.1% Triton X-100 was determined to be optimal as it sufficiently permeabilized the plasma and nuclear membrane to cause differential signal distribution (FIG. 14).

MCF7 cells were labeled with antibodies against position markers (sodium-potassium ATPase for plasma membrane, a-tubulin for cytoplasm, and histone H-2B for nucleus), and targeted with fluorescent localization signals (attached onto different MSNs) (FIG. 15a) and fluorescent DNA nanostructures (FIG. 15b). Fluorescence microscopy images of the labeled cells not only confirmed the specific localization of the position markers, but also showed the differential distribution of the localization signals in different subcellular compartments. Specifically, localization signal L1 could probe plasma membrane, cytoplasm and nucleus. 12 could probe plasma membrane and cytoplasm. L3 could only probe plasma membrane. The DNA nanostructure could probe plasma membrane, cytoplasm and nucleus. All cells were counterstained with nuclear dye Hoechst 33342.

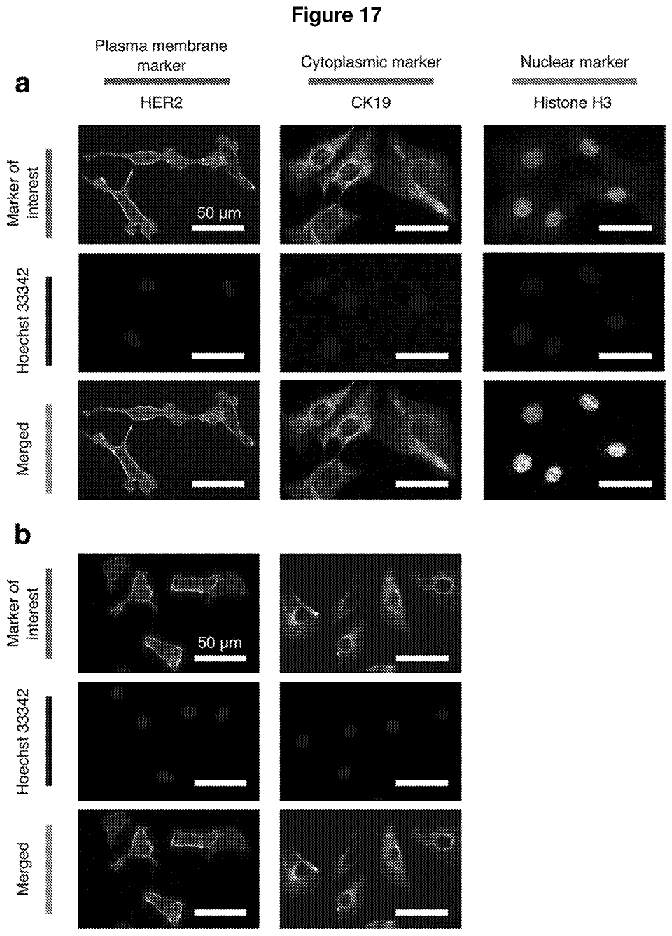

To validate this approach, the STAMP technology was applied to assay different markers of interest. The relative distributions of HER2, CK19 and histone H3, were first probed across subcellular compartments (FIG. 13c). These markers of interest are known to localize predominantly to the plasma membrane, cytoplasm and nucleus, respectively. Using SKOV3 cells, which express all these markers, the STAMP measurements were demonstrated to correlate well with fluorescence microscopy analysis (FIG. 17). Fluorescence microscopy images of target markers (HER2, CK19 and histone H3) in SKOV3 cells (FIG. 13c, Top panel) showed the markers' predominant localization in plasma membrane, cytoplasm and nucleus, respectively. STAMP barcodes were generated in the same cell line, quantified through qPCR and analyzed with matrix conversion (see (FIG. 13c, Bottom panel and FIG. 16 for details). STAMP analysis accurately reflected the markers' relative subcellular distributions. The accuracy of the STAMP technology was further verified to measure both marker expression levels and subcellular distributions (FIG. 13d). Various protein targets (HER2, CD44, CK19, histone H3 and S100P) were included which showed differential expressions across human cell lines. All STAMP signals were measured through standard qPCR analysis and normalized against appropriate IgG isotype controls (see Table 3). STAMP was used to measure various markers (HER2, CD44, CK19, histone H3 and S100P) in human cell lines with differential expression of these markers (i.e., MDAMB-231, SKBR3, SKOV3 and TIOSE6) (FIG. 13d, Top panel). Conventional methods were performed to determine the markers' expression levels (via flow cytometry) and subcellular distribution patterns (via immunofluorescence microscopy) (FIG. 13d, Bottom panel). In comparison to gold standard conventional methods, where flow cytometry was applied to quantify the marker expression levels and immunofluorescence microscopy to determine their subcellular distribution patterns, the STAMP measurements showed a good correlation (R2≥0.8812) across protein markers of different subcellular localizations.

TABLE 3
STAMP barcodes with their respective qPCR primer sets.
Antibody barcodes

| | | |
|---|---|---|
| SEQ ID NO. 12 | Ab- | ACGGGTATGATACTTCTATGATCGTACGAT |
| | Tetrahedron 1 | (5′ Thiol Modifier C6 S-S, 3′ 6-FAM) |
| SEQ ID NO. 13 | Ab- | ACGGGTATGATACTTTGCAATACGACTGTA |
| | Tetrahedron 2 | (5′ Thiol Modifier C6 S-S, 3′ 6-FAM) |
| SEQ ID NO. 14 | Ab- | ACGGGTATGATACTTAGTAACGTCTAGAAT |
| | Tetrahedron 3 | (5′ Thiol Modifier C6 S-S, 3′ 6-FAM) |
| SEQ ID NO. 15 | Ab- | ACGGGTATGATACTTTTGTAGCTATAGTTA |
| | Tetrahedron 4 | (5′ Thiol Modifier C6 S-S, 3′ 6-FAM) |

Localization labels

| | | |
|---|---|---|
| SEQ ID NO. 16 | L1 | AAGTATCATACCCGTCTCCACGACTTAGAATCAAAAAA |
| | | (5′ Phosphorylation) |
| SEQ ID NO. 17 | L2 | AAGTATCATACCCGTAGTTGCTGGACGATTGTAAAAAA |
| | | (5′ Phosphorylation, 3′ Thiol Modifier C3 S-S) |
| SEQ ID NO. 18 | L3 | AAGTATCATACCCGTTCACCGTAGCTCAATGGAAAAAA |
| | | (5′ Phosphorylation, 3′ Thiol Modifier C3 S-S) |

STAMP barcodes and qPCR primer sets

| | | |
|---|---|---|
| SEQ ID NO. 19 | Tetrahedron 1-L1 | ACGAACATTCCTAAGTCTGAAATTTATCACCCGCCATA |
| | | GTAGACGTATCACCAGGCAGTTGAGTTATCGTACGATC |
| | | ATAGAAGTATCATACCCGTCTCCACGACTTAGAATCAA |
| | | AAAA |
| SEQ ID NO. 20 | Forward | ATCACCCGCCATAGTAGACG |
| | primer | |
| SEQ ID NO. 21 | Reverse | GATTCTAAGTCGTGGAGACGG |
| | primer | |
| SEQ ID NO. 22 | Tetrahedron 1-L2 | ACGAACATTCCTAAGTCTGAAATTTATCACCCGCCATA |
| | | GTAGACGTATCACCAGGCAGTTGAGTTATCGTACGATC |
| | | ATAGAAGTATCATACCCGTAGTTGCTGGACGATTGTAA |
| | | AAAA |
| SEQ ID NO. 23 | Forward | ATCACCCGCCATAGTAGACG |
| | primer | |
| SEQ ID NO. 24 | Reverse | ACAATCGTCCAGCAACTACG |
| | primer | |
| SEQ ID NO. 25 | Tetrahedron 1-L3 | ACGAACATTCCTAAGTCTGAAATTTATCACCCGCCATA |
| | | GTAGACGTATCACCAGGCAGTTGAGTTATCGTACGATC |
| | | ATAGAAGTATCATACCCGTTCACCGTAGCTCAATGGAA |
| | | AAAA |
| SEQ ID NO. 26 | Forward | ATCACCCGCCATAGTAGACG |
| | primer | |
| SEQ ID NO. 27 | Reverse | CCATTGAGCTACGGTGAACG |
| | primer | |
| SEQ ID NO. 28 | Tetrahedron 2-L1 | ATTCAGACTTAGGAATGTTCGACATGCGAGGGTCCAAT |
| | | ACCGACGATTACAGCTTGCTACACGTTTACAGTCGTAT |
| | | TGCAAAGTATCATACCCGTCTCCACGACTTAGAATCAA |
| | | AAAA |
| SEQ ID NO. 29 | Forward | GACATGCGAGGGTCCAATAC |
| | primer | |
| SEQ ID NO. 30 | Reverse | GATTCTAAGTCGTGGAGACGG |
| | primer | |
| SEQ ID NO. 31 | Tetrahedron 2-L2 | ATTCAGACTTAGGAATGTTCGACATGCGAGGGTCCAAT |
| | | ACCGACGATTACAGCTTGCTACACGTTTACAGTCGTAT |
| | | TGCAAAGTATCATACCCGTAGTTGCTGGACGATTGTAA |
| | | AAAA |
| SEQ ID NO. 32 | Forward | GACATGCGAGGGTCCAATAC |
| | primer | |
| SEQ ID NO. 33 | Reverse | ACAATCGTCCAGCAACTACG |
| | primer | |
| SEQ ID NO. 34 | Tetrahedron 2-L3 | ATTCAGACTTAGGAATGTTCGACATGCGAGGGTCCAAT |
| | | ACCGACGATTACAGCTTGCTACACGTTTACAGTCGTAT |
| | | TGCAAAGTATCATACCCGTTCACCGTAGCTCAATGGAA |
| | | AAAA |
| SEQ ID NO. 35 | Forward | GACATGCGAGGGTCCAATAC |
| | primer | |
| SEQ ID NO. 36 | Reverse | CCATTGAGCTACGGTGAACG |
| | primer | |
| SEQ ID NO. 37 | Tetrahedron 3-L1 | ACTACTATGGCGGGTGATAAAACGTGTAGCAAGCTGTA |
| | | ATCGACGGGAAGAGCATGCCCATCCTTATTCTAGACGT |
| | | TACTAAGTATCATACCCGTCTCCACGACTTAGAATCAA |
| | | AAAA |
| SEQ ID NO. 38 | Forward | AGCTGTAATCGACGGGAAGA |
| | primer | |
| SEQ ID NO. 39 | Reverse | GATTCTAAGTCGTGGAGACGG |
| | primer | |
| SEQ ID NO. 40 | Tetrahedron 3-L2 | ACTACTATGGCGGGTGATAAAACGTGTAGCAAGCTGTA |
| | | ATCGACGGGAAGAGCATGCCCATCCTTATTCTAGACGT |
| | | TACTAAGTATCATACCCGTAGTTGCTGGACGATTGTAA |
| | | AAAA |
| SEQ ID NO. 41 | Forward | AGCTGTAATCGACGGGAAGA |
| | primer | |
| SEQ ID NO. 42 | Reverse | ACAATCGTCCAGCAACTACG |
| | primer | |
| SEQ ID NO. 43 | Tetrahedron 3-L3 | ACTACTATGGCGGGTGATAAAACGTGTAGCAAGCTGTA |
| | | ATCGACGGGAAGAGCATGCCCATCCTTATTCTAGACGT |
| | | TACTAAGTATCATACCCGTTCACCGTAGCTCAATGGAA |
| | | AAAA |
| SEQ ID NO. 44 | Forward | AGCTGTAATCGACGGGAAGA |
| | primer | |
| SEQ ID NO. 45 | Reverse | CCATTGAGCTACGGTGAACG |
| | primer | |
| SEQ ID NO. 46 | Tetrahedron 4-L1 | ACGGTATTGGACCCTCGCATGACTCAACTGCCTGGTG |
| | | ATACGAGGATGGGCATGCTCTTCCCGTTTAACTATAGC |
| | | TACAAAAGTATCATACCCGTCTCCACGACTTAGAATCA |
| | | AAAAA |
| SEQ ID NO. 47 | Forward | TCAACTGCCTGGTGATACGA |
| | primer | |
| SEQ ID NO. 48 | Reverse | GATTCTAAGTCGTGGAGACGG |
| | primer | |
| SEQ ID NO. 49 | Tetrahedron 4-L2 | ACGGTATTGGACCCTCGCATGACTCAACTGCCTGGTG |
| | | ATACGAGGATGGGCATGCTCTTCCCGTTTAACTATAGC |
| | | TACAAAAGTATCATACCCGTAGTTGCTGGACGATTGTA |
| | | AAAAA |
| SEQ ID NO. 50 | Forward | TCAACTGCCTGGTGATACGA |
| | primer | |
| SEQ ID NO. 51 | Reverse | ACAATCGTCCAGCAACTACG |
| | primer | |
| SEQ ID NO. 52 | Tetrahedron 4-L3 | ACGGTATTGGACCCTCGCATGACTCAACTGCCTGGTG |
| | | ATACGAGGATGGGCATGCTCTTCCCGTTTAACTATAGC |
| | | TACAAAAGTATCATACCCGTTCACCGTAGCTCAATGGA |
| | | AAAAA |
| SEQ ID NO. 53 | Forward | TCAACTGCCTGGTGATACGA |
| | primer | |
| SEQ ID NO. 54 | Reverse | CCATTGAGCTACGGTGAACG |
| | primer | |

### Example 5: Simultaneous Multiplexed Barcoding

Cell culture. All human cancer cell lines were obtained from American Type Culture Collection (ATCC). MDA-MB-231, SKBR3, SKOV3, CaOV3, OVCAR3, MCF7, and BT474 were grown in Dulbecco's modified essential medium (DMEM, Cellgro) supplemented with 10% fetal bovine serum (FBS, HyClone) and 1% penicillin-streptomycin (Cellgro). OV90, OVCA429, and UCI101 were cultured in RPMI-1640 medium (Cellgro) supplemented with 10% FBS and 1% penicillin streptomycin. HME1 (ATCC) was cultured in mammary epithelial cell growth medium (MEGM) BulletKit (Lonza). Normal ovarian surface epithelium (NOSE) cell lines were derived from ovarian surface epithelium (OSE) brushings and cultured in 1:1 mixture of MCDB 105 medium and Medium 199 (Sigma-Aldrich) with gentamicin (25 μg/ml) and 15% heat-inactivated serum. TIOSE4 and TIOSE6 cell lines were then obtained by transfecting NOSE cells with hTERT, and cultured in RPMI-1640 supplemented with 10% FBS and 1% penicillin-streptomycin. All cell lines were tested and free of mycoplasma contamination (MycoAlert Mycoplasma Detection Kit, Lonza, LT07-418).

DNA tetrahedron assembly and characterization. The four component DNA strands (Integrated DNA Technologies, IDT; Supplementary Table 1) were mixed in TEM buffer (10 mM Tris, 1 mM EDTA, 5 mM MgCl2) to a final concentration of 1 μM each, heated to 95° C. for 2 min, then cooled to room temperature over 2 h. Native polyacrylamide gel electrophoresis was conducted to confirm the formation of the DNA tetrahedron. 18 μl of FAM-labeled DNA nanostructure (0.1 μM) was mixed with 2 μl of BlueJuice gel loading buffer (Invitrogen). The mixture was loaded to a 6% TBE gel (Invitrogen), run at 200 V for 20 min and imaged using a ChemiDoc Touch imaging system (Bio-Rad). The hydrodynamic diameter of the formed DNA nanostructures was measured using a Zetasizer Nano ZS instrument (Malvern). The nanostructure morphology was confirmed using atomic force microscopy. Briefly, DNA nanostructures (10 μl, diluted to 5 nM in TEM buffer) were dropped onto freshly cleaved mica and allowed to air dry. Once dried, 50 μl TEM buffer was added and the sample was scanned using an SNL-10 probe (Bruker) on a Bioscope Catalyst AFM (Bruker).

Antibody-DNA conjugation. Antibodies (Table 2) were activated by mixing with sulfo-SMCC (Pierce) at 50-fold molar excess in PBS pH 7.4 with 1 mM EDTA and incubated for 2 h at room temperature. The reaction was buffer exchanged with Zeba micro spin desalting columns (Pierce) to remove excess sulfo-SMCC. DNA strands (short and long) modified with thiol and 6-FAM (200 μM, IDT; Supplementary Table 1) were activated by incubating with TCEP reducing gel (Pierce) to reduce the disulfide bonds for 1 h at room temperature. The reaction was then filtered and the gel washed several times to recover the activated DNA. The recovered DNA was concentrated using Amicon Ultra centrifugal filters (Millipore). The concentrations of the activated antibody and DNA were determined by absorbance measurements (Nanodrop, Thermo Fisher). The activated antibody was then mixed with excess activated DNA to a final concentration of 0.5 mg/ml and incubated overnight at 4° C. The reaction was filtered using Amicon Ultra centrifugal filters (100 kDa size cut-off, Millipore) and washed three times to remove unreacted DNA. The concentrations of the antibody-DNA conjugates were determined by BCA assay (Pierce) and the average numbers of DNA per antibody were estimated by fluorescence measurements (Spark 10M, Tecan). The hydrodynamic diameter and zeta potential of antibody and antibody-DNA conjugates were measured at 0.1 mg/ml using Zetasizer Nano ZS instrument (Malvern).

qPCR analysis. For qPCR analysis of STAMP barcodes, 2 μl of sample from the STAMP assay was mixed with primers (300 nM) in PowerUp SYBR Green Master Mix (Applied Biosystems). qPCR analysis was performed on a QuantStudio 5 real-time PCR system (Applied Biosystems) under fast cycling protocol recommended by the manufacturer: 50° C. for 2 min, 95° C. for 2 min, 40 cycles of 95° C. for 1 s and 60° C. for 30 s. The signal (cycle number) obtained from each marker was normalized against that of IgG isotype control. We validated the amplification efficiency of all qPCR primer pairs in amplifying their respective targets (Table 3), through a serial dilution of DNA templates (0.1 μM to 0.1 fM). To confirm the specificity of the primer pairs for their respective targets, 10 μM of each DNA template was subjected to qPCR with all primer sets as described above.

Next Generation Sequencing Analysis.

For multiplexed STAMP analysis, the sequencing library was prepared by two rounds of PCR amplification (see Table 4 for list of sequences used). In the first round, PCR using offset primers was performed to increase the sample complexity. Specifically, 10 μl of sample from the STAMP assay was mixed with the offset PCR primer mix, dNTPs, and Q5 high-fidelity DNA polymerase (New England Biolabs) to a final concentration of 400 nM, 0.5 mM, and 20 U/μL, respectively, in Q5 reaction buffer to a final volume of 25 μl. Thermal cycling was performed under the following cycling protocol: 5° C. for 3 min, 20 cycles of 95° C., 54° C., and 72° C. for 15 s each, followed by 72° C. for 3 min. The PCR reaction was cleaned up using Monarch PCR & DNA Cleanup Kit (New England Biolabs). The eluted product underwent another PCR amplification as previously described with indexed primers for 6 cycles. The PCR reaction was cleaned up using AMPure XP beads (Beckman Coulter), pooled and run in a lane of NextSeq (Illumina), SE 1×76 bp. Reads were mapped to a custom genome made up of all possible ligated barcode sequences, allowing 0 mismatches. The number of reads mapped to each marker was used as the expression signal and normalized against that of IgG isotype control.

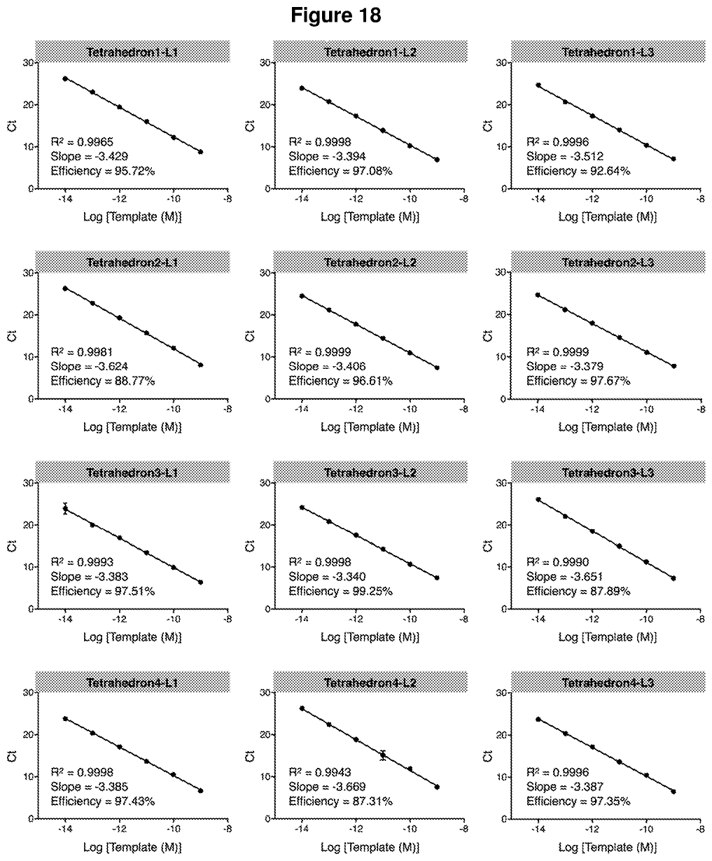

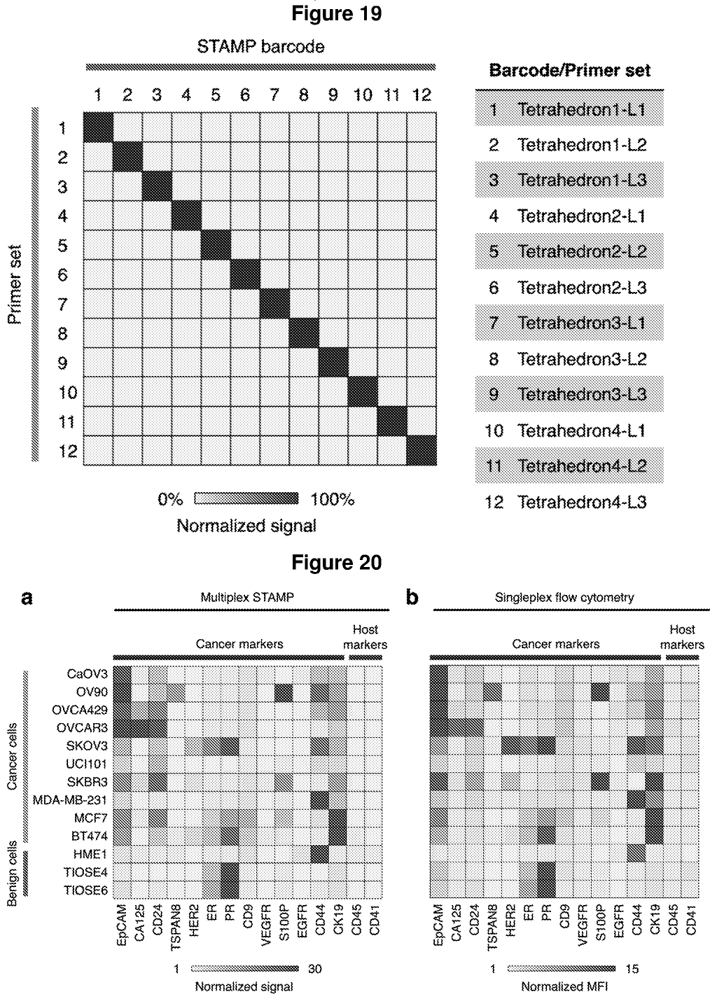

Next, the STAMP technology was developed to perform multiplexed, high-throughput protein measurements in cells. The platform's capacity was improved to distinguish different proteins through two approaches. First, by directly modifying the DNA sequences attached to the antibodies, templated STAMP barcodes were generated through nanostructure-assisted ligation of the variable tetrahedron overhangs. Second, the combinatorial arrangement of the sequence subunits in the tetrahedral core (FIG. 1a) were leveraged to further improve the specificity of barcode differentiation, especially in qPCR analysis (e.g., design of primers). To validate this strategy, STAMP barcodes were designed, generated, and tested with their respective primer pairs (Table 3). All barcodes could be efficiently amplified (FIG. 18) and showed minimal crosstalk for simultaneous multiplexing analysis (FIG. 19).

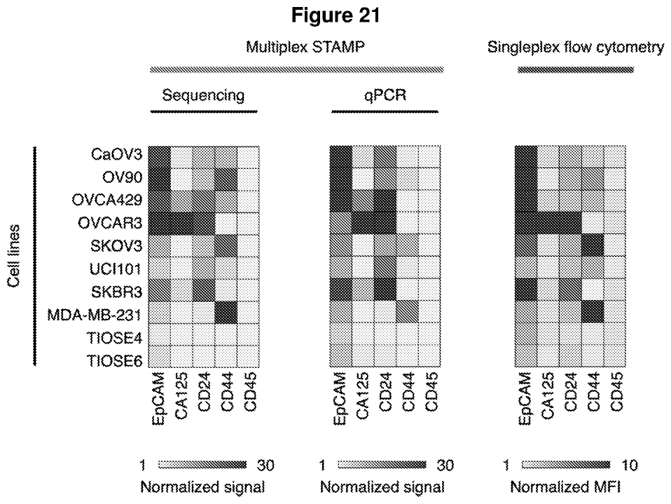

The multiplexed STAMP platform was employed for one-pot protein analysis through next generation sequencing (Table 4). On the basis of prior studies, the following putative cancer markers: EpCAM, CA125, CD24, TSPAN8, HER2, ER, PR, CD9, VEGFR, S100P, EGFR, CD and CK19; were selected and host markers CD45 and CD4 (Table 2). Multiplexed STAMP analysis was performed on various human cancer cell lines as well as benign cell lines (FIG. 20a). Putative cancer markers (EpCAM, CA125, CD24, TSPAN8, HER2, ER, PR, CD9, VEGFR, S100P, EGFR, CD and CK19) and host cell markers (CD45 and CD4) were measured in 10 human cancer cell lines and 3 benign cell lines. The protein markers have various subcellular localizations (e.g., plasma membrane, cytoplasm and nucleus). All protein measurements were performed by multiplex STAMP, through simultaneous STAMP barcode generation and next generation sequencing analysis (FIG. 20a), and singleplex flow cytometry, where fluorescent antibody measurements were made one at a time (FIG. 20b). In comparison to conventional singleplex flow cytometry (FIG. 20b), the STAMP measurements not only showed a good agreement but also demonstrated capacity for massively parallel sequencing analysis (FIG. 21).

TABLE 4
Primers and index sequences for next generation sequencing.
Sequencing library preparation primers

| | | |
|---|---|---|
| SEQ ID NO. 55 | Round 1 Read | ACACTCTTTCCCTACACGACGCTCTTCCGATCTNNN |
| | 1 Tetrahedron | NNNCCCGCCATAGTAGACGTATC |
| | 1 A | (N denotes a random base) |
| SEQ ID NO. 56 | Round 1 Read | ACACTCTTTCCCTACACGACGCTCTTCCGATCTNNN |
| | 1 Tetrahedron | NNNCGCCATAGTAGACGTATCACC |
| | 1 B | |
| SEQ ID NO. 57 | Round 1 Read | ACACTCTTTCCCTACACGACGCTCTTCCGATCTNNN |
| | 1 Tetrahedron | NNNGCCATAGTAGACGTATCACCAG |
| | 1 C | |
| SEQ ID NO. 58 | Round 1 Read | ACACTCTTTCCCTACACGACGCTCTTCCGATCTNNN |
| | 1 Tetrahedron | NNNTAGTAGACGTATCACCAGGCA |
| | 1 D | |
| SEQ ID NO. 59 | Round 1 Read | ACACTCTTTCCCTACACGACGCTCTTCCGATCTNNN |
| | 1 Tetrahedron | NNNAGTAGACGTATCACCAGGCA |
| | 1 E | |
| SEQ ID NO. 60 | Round 1 Read | ACACTCTTTCCCTACACGACGCTCTTCCGATCTNNN |
| | 1 Tetrahedron | NNNGGGTCCAATACCGACGATTA |
| | 2A | |
| SEQ ID NO. 61 | Round 1 Read | ACACTCTTTCCCTACACGACGCTCTTCCGATCTNNN |
| | 1 Tetrahedron | NNNGGTCCAATACCGACGATTACA |
| | 2 B | |
| SEQ ID NO. 62 | Round 1 Read | ACACTCTTTCCCTACACGACGCTCTTCCGATCTNNN |
| | 1 Tetrahedron | NNNTCCAATACCGACGATTACAGC |
| | 2 C | |
| SEQ ID NO. 63 | Round 1 Read | ACACTCTTTCCCTACACGACGCTCTTCCGATCTNNN |
| | 1 Tetrahedron | NNNCAATACCGACGATTACAGCTTG |
| | 2 D | |
| SEQ ID NO. 64 | Round 1 Read | ACACTCTTTCCCTACACGACGCTCTTCCGATCTNNN |
| | 1 Tetrahedron | NNNTACCGACGATTACAGCTTGC |
| | 2 E | |
| SEQ ID NO. 65 | Round 1 Read | ACACTCTTTCCCTACACGACGCTCTTCCGATCTNNN |
| | 1 Tetrahedron | NNNCAAGCTGTAATCGACGGGAA |
| | 3 A | |
| SEQ ID NO. 66 | Round 1 Read | ACACTCTTTCCCTACACGACGCTCTTCCGATCTNNN |
| | 1 Tetrahedron | NNNAGCTGTAATCGACGGGAAG |
| | 3 B | |
| SEQ ID NO. 67 | Round 1 Read | ACACTCTTTCCCTACACGACGCTCTTCCGATCTNNN |
| | 1 Tetrahedron | NNNCTGTAATCGACGGGAAGAGC |
| | 3 C | |
| SEQ ID NO. 68 | Round 1 Read | ACACTCTTTCCCTACACGACGCTCTTCCGATCTNNN |
| | 1 Tetrahedron | NNNGTAATCGACGGGAAGAGCAT |
| | 3 D | |
| SEQ ID NO. 69 | Round 1 Read | ACACTCTTTCCCTACACGACGCTCTTCCGATCTNNN |
| | 1 Tetrahedron | NNNAATCGACGGGAAGAGCATG |
| | 3 E | |
| SEQ ID NO. 70 | Round 1 Read | ACACTCTTTCCCTACACGACGCTCTTCCGATCTNNN |
| | 1 Tetrahedron | NNNCCTGGTGATACGAGGATGG |
| | 4 A | |
| SEQ ID NO. 71 | Round 1 Read | ACACTCTTTCCCTACACGACGCTCTTCCGATCTNNN |
| | 1 Tetrahedron | NNNCTGGTGATACGAGGATGGG |
| | 4 B | |
| SEQ ID NO. 72 | Round 1 Read | ACACTCTTTCCCTACACGACGCTCTTCCGATCTNNN |
| | 1 Tetrahedron | NNNGTGATACGAGGATGGGCAT |
| | 4 C | |
| SEQ ID NO. 73 | Round 1 Read | ACACTCTTTCCCTACACGACGCTCTTCCGATCTNNN |
| | 1 Tetrahedron | NNNTGATACGAGGATGGGCATG |
| | 4 D | |
| SEQ ID NO. 74 | Round 1 Read | ACACTCTTTCCCTACACGACGCTCTTCCGATCTNNN |
| | 1 Tetrahedron | NNNATACGAGGATGGGCATGC |
| | 4 E | |
| SEQ ID NO. 75 | Round 1 Read | GTGACTGGAGTTCAGACGTGTGCTCTTCCGATCTCG |
| | 2 Label | TGGAGACGGGTATGATAC |
| SEQ ID NO. 76 | Round 2 Read | AATGATACGGCGACCACCGAGATCTACACTCTTTCC |
| | 1 | CTACACGAC |
| SEQ ID NO. 77 | Round 2 Read | CAAGCAGAAGACGGCATACGAGATXXXXXXGTGACT |
| | 2 | GGAGTTCAGACGT |
| | | (see below for the 6-base index sequences) |

Index sequences

| | | |
|---|---|---|
| SEQ ID NO. 78 | Index 1 | CGTGAT |
| SEQ ID NO. 79 | Index 2 | ACATCG |
| SEQ ID NO. 80 | Index 3 | GCCTAA |
| SEQ ID NO. 81 | Index 4 | TGGTCA |
| SEQ ID NO. 82 | Index 5 | CACTGT |
| SEQ ID NO. 83 | Index 6 | ATTGGC |
| SEQ ID NO. 84 | Index 7 | GATCTG |
| SEQ ID NO. 85 | Index 8 | TCAAGT |
| SEQ ID NO. 86 | Index 9 | CTGATC |
| SEQ ID NO. 87 | Index 10 | AAGCTA |
| SEQ ID NO. 88 | Index 11 | GTAGCC |
| SEQ ID NO. 89 | Index 12 | TACAAG |

### Example 6: STAMP for Clinical Rare Cell Measurements

DNA tetrahedron assembly and characterization. The four component DNA strands (Integrated DNA Technologies, IDT; Table 1) were mixed in TEM buffer (10 mM Tris, 1 mM EDTA, 5 mM MgCl2) to a final concentration of 1 μM each, heated to 95° C. for 2 min, then cooled to room temperature over 2 h. Native polyacrylamide gel electrophoresis was conducted to confirm the formation of the DNA tetrahedron. 18 μl of FAM-labeled DNA nanostructure (0.1 μM) was mixed with 2 μl of BlueJuice gel loading buffer (Invitrogen). The mixture was loaded to a 6% TBE gel (Invitrogen), run at 200 V for 20 min and imaged using a ChemiDoc Touch imaging system (Bio-Rad). The hydrodynamic diameter of the formed DNA nanostructures was measured using a Zetasizer Nano ZS instrument (Malvern). The nanostructure morphology was confirmed using atomic force microscopy. Briefly, DNA nanostructures (10 μl, diluted to 5 nM in TEM buffer) were dropped onto freshly cleaved mica and allowed to air dry. Once dried, 50 μl TEM buffer was added and the sample was scanned using an SNL-10 probe (Bruker) on a Bioscope Catalyst AFM (Bruker).

Antibody-DNA conjugation. Antibodies (Table 2) were activated by mixing with sulfo-SMCC (Pierce) at 50-fold molar excess in PBS pH 7.4 with 1 mM EDTA and incubated for 2 h at room temperature. The reaction was buffer exchanged with Zeba micro spin desalting columns (Pierce) to remove excess sulfo-SMCC. DNA strands (short and long) modified with thiol and 6-FAM (200 μM, IDT; Supplementary Table 1) were activated by incubating with TCEP reducing gel (Pierce) to reduce the disulfide bonds for 1 h at room temperature. The reaction was then filtered and the gel washed several times to recover the activated DNA. The recovered DNA was concentrated using Amicon Ultra centrifugal filters (Millipore). The concentrations of the activated antibody and DNA were determined by absorbance measurements (Nanodrop, Thermo Fisher). The activated antibody was then mixed with excess activated DNA to a final concentration of 0.5 mg/ml and incubated overnight at 4° C. The reaction was filtered using Amicon Ultra centrifugal filters (100 kDa size cut-off, Millipore) and washed three times to remove unreacted DNA. The concentrations of the antibody-DNA conjugates were determined by BCA assay (Pierce) and the average numbers of DNA per antibody were estimated by fluorescence measurements (Spark 10M, Tecan). The hydrodynamic diameter and zeta potential of antibody and antibody-DNA conjugates were measured at 0.1 mg/ml using Zetasizer Nano ZS instrument (Malvern).

The study was approved by the National University Hospital (NUH) Institutional Review Board (2014/01088). All subjects were recruited according to IRB-approved protocols after obtaining informed consent. A total of 69 samples (breast FNA biopsies) were collected for this study (Table 5). For training the regression model, patient-matched cancer and normal samples were used (n=34). Additional samples (n=35) were used to validate the model. Clinical diagnoses (including molecular subtyping and clinical grading) were established from gold standard pathology reports. All FNA samples were anonymized and STAMP experiments were conducted blinded from the clinical results.

To evaluate the clinical utility of the STAMP platform for rare cell analysis, a feasibility study was conducted using breast cancer as a model. the following questions were addressed:
(1) if STAMP could be directly applied to scarce clinical specimens for multiplexed protein analysis,
(2) how accurate is STAMP in detecting cancer,
(3) if STAMP signatures could distinguish additional cancer characteristics (i.e., molecular subtypes and aggressiveness).

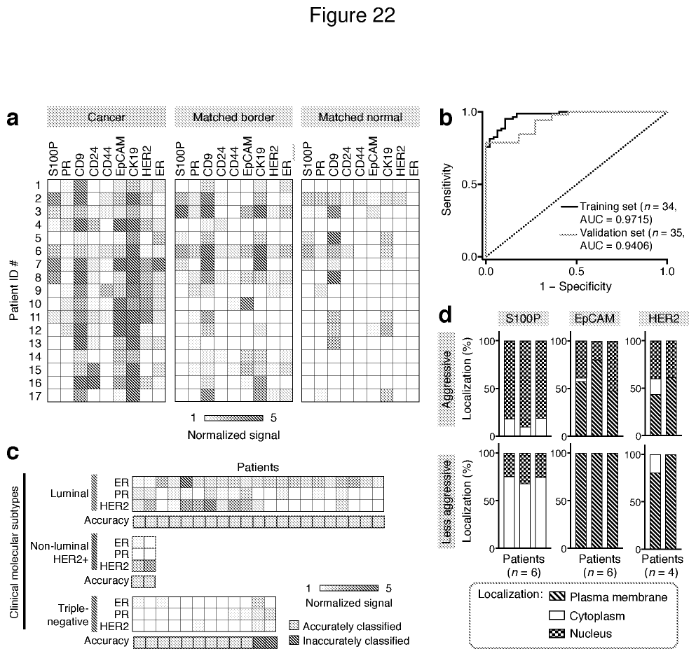

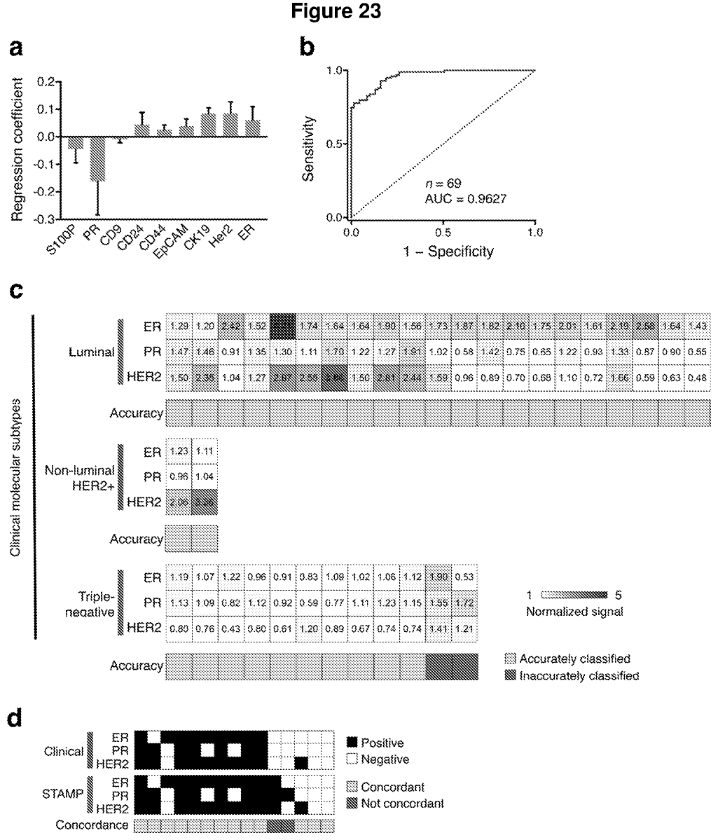

Breast fine needle aspiration (FNA) biopsies (n=69; Table 5) were obtained and employed in the microfluidic platform (FIG. 1b) to perform multiplexed STAMP analysis directly on these clinical specimens. For 17 of the breast cancer patients, FNAs were obtained from the cancer site, as well as patient-matched cancer border and normal tissue (FIG. 22a). Using the STAMP measurements of these patient-matched cancer and normal FNAs, a cross-trained scoring model was developed based on linear regression (FIG. 23a) and validated the model using leave-one-out cross-validation as well as additional FNAs not used in training the model (FIG. 22b and FIG. 23b). In comparison to gold-standard pathology of corresponding surgical tissues, the STAMP analysis demonstrated a high accuracy to diagnose cancer (FIG. 22b, AUC=0.9715 for the training cohort and AUC=0.9406 for the validation set). Furthermore, based on the expression analysis of ER, PR and HER2, the STAMP platform could classify the cancer samples into distinct molecular subtypes (i.e., luminal, non-luminal HER2-positive, and triple negative) according to established criteria, and demonstrated >94% subtyping accuracy as compared to pathology reports (FIG. 22c, FIG. 23c-d). Finally, subcellular protein distribution could be informative about additional clinical features. Specifically, in agreement with published histopathology studies on the protein markers S100P, EpCAM and HER2, the multiplexed STAMP analysis revealed that a higher nuclear localization of these markers could correspond to more aggressive cancer phenotypes (FIG. 22d). In comparison to cancer samples which showed less aggressive clinical features (clinical grade 1 and 2), the more aggressive samples (clinical grade 3) demonstrated a higher nuclear localization of several markers (i.e., S100P, EpCAM and HER2) (FIG. 22d).

In developing the regression model, STAMP measurements from only the cancer and patient-matched normal FNAs were used. The STAMP measurements were used as the predictor variables, and categorical FNA status (cancer vs. normal) as the outcome variable in linear regression (FIG. 23a). To determine the model's performance and avoid overfitting, leave-one-out cross-validation was performed. The averaged regression coefficients from the cross-validation were used to create a linear regression model. Across all clinical specimens tested (n=69, training and validation cohorts), the STAMP analysis showed a high assay accuracy to diagnose cancer (AUC=0.9627) (FIG. 23b).

TABLE 5
Clinical information.

| Characteristic | Number (%) | |
|---|---|---|
| Total breast tissue samples | 69 | |
| Breast cancer | 35 | (50.72%) |
| Matched normal | 17 | (24.64%) |
| Matched border | 17 | (24.64%) |

Age

| | |
|---|---|
| Median | 59.5 |
| Range | 23-89.5 |

BMI

| | |
|---|---|
| Median | 24.5 |
| Range | 19.7-41.6 |

Menopausal status

| | | |
|---|---|---|
| Pre-menopause | 19 | (54.29%) |
| Post-menopause | 16 | (45.71%) |

Cancer types

| | | |
|---|---|---|
| Ductal carcinoma in situ | 22 | (62.86%) |
| Invasive ductal carcinoma | 9 | (25.71%) |
| Invasive lobular carcinoma | 2 | (5.71%) |
| Others | 2 | (5.71%) |

Cancer molecular subtypes

| | | |
|---|---|---|
| Luminal | 21 | (60.00%) |
| Non-luminal HER2+ | 2 | (5.71%) |
| Triple-negative | 12 | (34.29%) |

Cancer grade

| | | |
|---|---|---|
| 1-2 | 13 | (37.14%) |
| 3 | 22 | (62.86%) |

### Example 7: Comparison with Existing Methods and Systems

When implemented on a miniaturized microfluidic device for clinical applications, STAMP enabled multiplexed protein typing and subcellular distribution analysis in scant patient samples. The STAMP-revealed signatures not only accurately classified cancer molecular subtypes, but also provided new measurements of disease aggressiveness.

Motivated by the DNA sequence-structure synergy, the STAMP technology was developed as a 3D barcoding platform. As compared to existing protein detection technologies, the STAMP platform shows distinct advantages, with respect to both assay format and assay performance (Table 6).

TABLE 6
Comparison of protein detection technologies.

| | | | | | | | Multiplex |
|---|---|---|---|---|---|---|---|
| | | | | | | | Immune- |
| | | | | | Western | Fluorescence | histochemistry |
| | STAMP | Immuno-PCR | NanoString | ELISA | blotting | microscopy | (IHC) |
| Assay format | Solution | Solution | Solution | Solution | Solution | Localized microscopy | Localized microscopy |
| Antibody | Conjugated | Conjugated | Conjugated | Antibody | Antibody | Conjugated (Ab-long | Antibody |
| format | antibody (Ab- | antibody (Ab- | antibody (Ab- | | | DNA) or Antibody | |
| | short DNA) | long DNA) | long DNA) | | | | |
| Readout | qPCR or | qPCR or | Fluorescence | Chemi- | Chemi- | Fluorescence imaging | Immunohistochemistry |
| format | sequencing | sequencing | imaging | luminescence | luminescence | | imaging |
| Detection | 10⁻²² mol | 10⁻¹⁹-10⁻²¹ | 10⁻²⁰ mol | 10⁻¹⁶-10⁻¹⁷ | 10⁻¹⁴-10⁻¹⁵ | Ab-long DNA: 10⁻¹⁷- | less sensitive |
| limit | proteins | mol proteins¹ | proteins² | mol proteins⁴ | mol proteins⁵ | 10⁻¹⁹ mol proteins⁶ | than western blotting⁷ |
| | single cell | single cell | single cell³ | | | Ab: less sensitive | semi-quantitative |
| | | | | | | than western blotting⁷ | |
| Multiplexing | High | High | High | Low | Low | Moderate | Moderate |
| capability | | | | | | | |
| Throughput | High | High | High | Moderate | Low | Low | Low |
| Time taken | ~2 hours (on | More than 2 | More than 12 | More than 6 | More than 12 | More than 6 hours | More than 12 |
| | chip) | hours (>2 h) | hours (>12 h - | hours (>6 h) | hours (>12 h - | (>6 h) | hours (>12 h - |
| | | | overnight | | overnight | | repeated striping |
| | | | incubation) | | incubation) | | and labelling) |
| Suitable for | YES | YES | YES | NO | NO | NO | NO |
| rare cell | | | | | | | |
| analysis | | | | | | | |
| Subcellular | YES | NO | NO | NO | NO | YES | YES |
| localization | | | | | | | |
| analysis | | | | | | | |

References:
¹Niemeyer, C. M., et al. Trends Biotechnol 23, 208-216 (2005).
²nCounter Analysis System Product Data Sheet (NanoString Tech). URL: http://www.biosystems.com.ar/archivos/folletos/165/PDS_nCounter_System.pdf
³Ullal, A. V. et al. Sci Transl Med 6, 219ra9 (2014).
⁴Giljohann, D. A. & Mirkin, C. A. Nature 462, 461-464 (2009).
⁵Halenbeck, R. et al. J Biol Chem 265, 21922-21928 (1990).
⁶Klaesson, A. et al. Sci Rep 8, 5400 (2018).
⁷A guide to protein detection (Abcam). URL: https://docs.abcam.com/pdf/proteins/a-guide-to-protein-detection.pdf

Real-time binding kinetics. The binding kinetics of the antibody-DNA conjugates were measured by bio-layer interferometry (Pall Fortebio). Briefly, protein antigens (e.g., HER2, Acro Biosystems) were immobilized onto streptavidin-functionalized interferometry sensors. After a brief washing step, the loaded biosensors were dipped into 5 μg/ml solutions of conjugated antibodies and incubated for 300 s to measure different associated antibody binding. This was followed by another washing step. All binding data (changes in optical thickness of the biolayer) were measured as wavelength shifts, in a continuous manner, to determine binding kinetics.

STAMP barcode generation (tube format). For subcellular marker expression and distribution analysis, cells were fixed and permeabilized in 4% formaldehyde with 0.1% Triton X-100 (Sigma-Aldrich), to facilitate differential distributions of the localization labels across subcellular compartments (see below). Cell suspensions were mixed with the prepared antibody-DNA conjugates (5 μg/ml each), incubated for 1 h at 4° C. and washed through conventional micro-centrifugation (300 g, 5 min). The cells were then re-suspended in the STAMP assay mix containing 0.5 μM DNA tetrahedron probes and localization labels, and reaction was incubated for 1 h at room temperature.

Ligation

For enzymatic ligation, T4 DNA ligase (New England Biolabs) was added in ligase buffer to a final concentration of 20 U/μL, and incubated for 10 min at room temperature. Upon heat inactivation of ligase (65° C., 10 min), STAMP barcodes were liberated for further analysis without additional purification. All experiments included an intrinsic control for data normalization, through simultaneous incubation of IgG isotype control antibody-DNA conjugates.

Click ligation. For DNA click ligation, the tetrahedron variable ends (3′-end) were modified with azide while the short localization labels were modified with alkyne at the 5′-end (IDT; Table 1). The STAMP reaction was incubated in the presence of copper (II) sulfate (Sigma-Aldrich, 100 μM), tris(3-hydroxypropyltriazolylmethyl)amine (THPTA, Lumiprobe, 700 μM) and sodium ascorbate (Sigma-Aldrich, 1 mM) in 0.2 M sodium chloride for 2 h at room temperature. The collected barcodes were buffer exchanged to water with Zeba micro spin desalting columns (Pierce) to remove excess reagents for further analysis.

Localization identifiers: Mesoporous silica nanoparticle (MSN) preparation and application.

To prepare the small MSNs, 2 g of cetyltrimethylammonium bromide (CTAB, Sigma-Aldrich) and 40 μl of triethanolamine (TEA, Sigma-Aldrich) were dissolved in 20 mL of water and heated to 90° C. 1.5 ml of tetraethyl orthosilicate (TEOS, Sigma-Aldrich) was then added and allowed to react for 15 min, followed by addition of 300 μl (3-aminopropyl)triethoxysilane (APTES, Sigma-Aldrich). The solution was allowed to react for 1 h. To prepare the large MSNs, 50 mg of CTAB and 14 mg of sodium hydroxide were dissolved in 25 mL of water and heated to 80° C., followed by addition of 250 μl TEOS. After 1 h, 50 μl of TEOS and 50 μl of APTES were added to the mixture and allowed to react for another hour. The formed MSNs were washed with ethanol and water, refluxed in methanol overnight, and finally resuspended in PBS. Transmission electron microscopy (TEM) images of the MSNs were obtained using a JEM-2010F TEM (JEOL).

To conjugate the MSNs with DNA localization labels, the particles were activated with 2 mM sulfo-SMCC (Pierce), and washed to remove excess reagents. Localization DNA labels, modified with thiol (200 μM, IDT; Table 3), were activated as previously described, and added to the MSNs. The reaction was incubated overnight at 4° C., and filtered using Amicon Ultra centrifugal filters (Millipore) to remove unbound DNA. To investigate marker subcellular distribution, STAMP was performed as previously described, by incubating targeted cells with a mixture of DNA nanostructures and MSN-conjugated localization labels simultaneously. STAMP barcodes containing localization information were measured and data analysis (see FIG. 16 for details) was performed using the R-package (version 3.5.0).

qPCR analysis. For qPCR analysis of STAMP barcodes, 2 μl of sample from the STAMP assay was mixed with primers (300 nM) in PowerUp SYBR Green Master Mix (Applied Biosystems). qPCR analysis was performed on a QuantStudio 5 real-time PCR system (Applied Biosystems) under fast cycling protocol recommended by the manufacturer: 50° C. for 2 min, 95° C. for 2 min, 40 cycles of 95° C. for 1 s and 60° C. for 30 s. The signal (cycle number) obtained from each marker was normalized against that of IgG isotype control. We validated the amplification efficiency of all qPCR primer pairs in amplifying their respective targets (Table 3), through a serial dilution of DNA templates (0.1 μM to 0.1 fM). To confirm the specificity of the primer pairs for their respective targets, 10 μM of each DNA template was subjected to qPCR with all primer sets as described above.

Next Generation Sequencing Analysis.

For multiplexed STAMP analysis, the sequencing library was prepared by two rounds of PCR amplification (see Table 4 for list of sequences used). In the first round, PCR using offset primers was performed to increase the sample complexity. Specifically, 10 μl of sample from the STAMP assay was mixed with the offset PCR primer mix, dNTPs, and Q5 high-fidelity DNA polymerase (New England Biolabs) to a final concentration of 400 nM, 0.5 mM, and 20 U/μL, respectively, in Q5 reaction buffer to a final volume of 25 μl. Thermal cycling was performed under the following cycling protocol: 5° C. for 3 min, 20 cycles of 95° C., 54° C., and 72° C. for 15 s each, followed by 72° C. for 3 min. The PCR reaction was cleaned up using Monarch PCR & DNA Cleanup Kit (New England Biolabs). The eluted product underwent another PCR amplification as previously described with indexed primers for 6 cycles. The PCR reaction was cleaned up using AMPure XP beads (Beckman Coulter), pooled and run in a lane of NextSeq (Illumina), SE 1×76 bp. Reads were mapped to a custom genome made up of all possible ligated barcode sequences, allowing 0 mismatches. The number of reads mapped to each marker was used as the expression signal and normalized against that of IgG isotype control.

Evaluation of STAMP Performance.

To compare the performance of the STAMP assay with direct long DNA conjugates (Ab-long DNA), standard polystyrene beads with known binding capacity were used to ensure uniformity and enable validation through flow cytometry. HER2-modified beads were prepared by incubating streptavidin-coated 3.0 μm polystyrene beads (Spherotech) in 10 μg/ml biotinylated HER2 (Acro Biosystems) in PBS with 0.5% bovine serum albumin (BSA, Sigma) overnight at 4° C. The mixture was then centrifuged, washed, and resuspended in PBS with 0.5% BSA. The modified beads were subjected to the STAMP assay and targeting with the direct long DNA conjugates, before being analyzed with qPCR as previously described. All antibody binding was also cross-validated with flow cytometry. To assess the sensitivity of the STAMP assay for cell measurements, cell suspensions were prepared, counted using a Countess II automated cell counter (Invitrogen), before being serially diluted and subjected to the STAMP assay as previously described.

Flow Cytometry.

Cell suspensions were prepared and labeled with 5 μg/ml primary antibodies for 1 h at 4° C., as previously described. Following centrifugation and washing, cells were labeled with 2 μg/ml FITC-conjugated secondary antibody (Becton Dickinson) for 30 min at 4° C. and washed twice by centrifugation. FITC fluorescence was assessed using a LSRII flow cytometer (Becton Dickinson). Mean fluorescence intensity of all cells, excluding debris, was determined using FlowJo (version 10.4.2), and biomarker expression levels were normalized against isotype control antibodies.

Immunofluorescence.

To measure subcellular protein localization and distribution, cells were cultured on a 8-well chamber slide (Nunc). Cells were fixed and permeabilized, as previously described, and blocked with 5% BSA for 1 h at room temperature. The prepared cells were labelled with 10 μg/ml of primary antibodies overnight at 4° C., washed twice with PBS, and then incubated with 2 μg/ml secondary antibodies for 1 h at room temperature. All cells were also stained with nuclear dye Hoechst 33342 (Molecular Probes) for 5 min at room temperature, before being mounted (Vector Laboratories). Fluorescence images were acquired using a Leica TCS SP8 confocal microscope (Leica Microsystems) at 20× magnification.

Statistical Analysis.

Unless otherwise stated, all measurements were performed in triplicates, and the data are presented as mean±standard deviation. For the size and zeta potential analyses, we performed the non-parametric Kruskal-Wallis tests to detect differences for three or more groups of measurements. This was followed by multiple comparisons corrected by controlling False Discovery Rate (Benjamin, Krieger and Yekutieli method). q<0.05 was determined as significant. For the STAMP data analysis, Shapiro-Wilk tests were performed to evaluate data normality. ANOVA was performed to detect differences for three or more groups of measurements. For inter sample comparisons, multiple pairs of samples were each tested via two-tailed Student's t-test, and the resulting P values were adjusted for multiple hypothesis testing using Bonferroni correction. P<0.05 was determined as significant. For developing the regression model, STAMP measurements from only the patient-matched cancer and normal FNAs were used. The categorical cancer and normal samples were recoded to a binary scale to use it as the outcome variable in linear regression. The STAMP expression values of all markers were used as the predictor variables. To determine performance of the model and avoid overfitting, leave-one-out cross validation was performed on the samples. The averaged regression coefficients from the cross validation were then used to create a linear regression model. This regression model was applied and validated for scoring all FNA samples (training set and validation set). Receiver operating characteristic (ROC) curve was generated from all patient profiling data and constructed by plotting sensitivity versus (1−specificity), and the value of area under the curve (AUC) was computed using the trapezoidal rule. Detection sensitivity, specificity and accuracy were calculated using standard formulas. Statistical analysis was performed using the R-package (version 3.5.0) and GraphPad Prism (version 7.0c).

1. A method for detecting and/or identifying a target in a sample comprising:
forming a modified moiety by conjugating a nucleic acid sequence to a moiety directed to the target;
forming a nucleic acid nanostructure comprising a segment sequence complementary to a portion of the nucleic acid sequence of the modified moiety,
wherein the nucleic acid nanostructure comprises a tetrahedron;
incubating the sample with the modified moiety to form a complex between the modified moiety and the target;
removing modified moieties that do not form a complex with the target;
allowing the complementary segment sequence of the nanostructure to hybridize to the portion of the nucleic acid sequence of the modified moiety to which it is complementary;
forming a nucleic acid barcode comprising the complementary segment sequence of the nanostructure; and
detecting the nucleic acid barcode, whereby the detection of the nucleic acid barcode indicates that the target is present in the sample.

2. The method according to claim 1, wherein the moiety is an antibody and the target is a target protein.

3-4. (canceled)

5. The method according to claim 1, wherein a plurality of modified moieties directed to a plurality of targets and a plurality of nucleic acid nanostructures comprising a segment sequence complementary to a portion of each of the plurality of nucleic acid sequences of the modified moieties are formed.

6. The method according to claim 1, further comprising forming at least two different location identifiers wherein each location identifier comprises a nucleic acid sequence comprising a segment sequence complementary to a second portion of the nucleic acid sequence of the modified moiety and a unique identifier that can be used to determine the cell or subcellular location of the target when the location identifiers bind to the second portion of the nucleic acid sequence of the modified moiety, wherein the unique identifier comprises a nucleic acid sequence.

7. (canceled)

8. The method according to claim 6, wherein further comprising permeabilizing a first cell sample with a first permeabilization buffer to allow a first location identifier to enter a first subcellular location of the cell and permeabilizing a second cell sample with a second permeabilization buffer to allow a second location identifier to enter a second subcellular location of the cell wherein the first and second subcellular location are not the same and detection of the first, or second location identifier indicates the amount of the target present in different cellular locations.

9. The method according to claim 6, wherein the at least two different location identifiers comprises at least three different location identifiers, a first location identifier wherein the unique identifier comprises a nucleic acid sequence, a second location identifier wherein the unique identifier comprises a nucleic acid sequence conjugated to a nanoparticle having a diameter of 60 nm or less and a third location identifier wherein the unique identifier comprises a nucleic acid sequence conjugated to a nanoparticle having a diameter of 100 nm or more wherein detection of the first, second or third location identifier indicates where in the cellular environment the target was present.

10. The method according to claim 6, further comprising ligating the complementary segment sequence of the nanostructure with the complementary segment sequence of the location identifier; forming a nucleic acid barcode comprising the ligated complementary segment sequence of the nanostructure and the complementary segment sequence of the location identifier.

11. The method according to claim 6, further comprising:
determining the relative location of at least two reference target proteins, each having a different known cellular distribution, with a set of interacting nucleic acid structures comprising i) a modified antibody having a nucleic acid sequence conjugated thereto directed to each reference target protein; ii) a nucleic acid nanostructure comprising a segment sequence complementary to a portion of the nucleic acid sequence conjugated to the modified antibody; and iii) a unique identifier by detecting at least two reference barcodes formed comprising the complementary segment sequence of the nanostructure, the complementary segment sequence of the second portion of the nucleic acid sequence of the modified antibody and a unique identifier;
determining the relative amount and/or location of at least one target protein with a set of interacting nucleic acid structures comprising i) a modified moiety having a nucleic acid sequence conjugated thereto directed to each target protein; ii) a nucleic acid nanostructure comprising a segment sequence complementary to a portion of the nucleic acid sequence conjugated to the modified moiety; and iii) a unique identifier, by detecting at least one barcode comprising the complementary segment sequence of the nanostructure, the complementary segment sequence of the second portion of the nucleic acid sequence of the modified moiety and the unique identifier;
analyzing the at least two reference barcodes formed when the reference target proteins are present and comparing the relative distribution of the at least two reference barcodes with the at least one barcode formed when the target protein is present and determining the relative cellular location of the target protein.

12. The method according to claim 11, wherein the relative cellular location of the target protein is determined via matrix conversion.

13. The method according to claim 6, wherein the nanoparticle comprises mesoporous silica nanoparticle.

14. The method according to claim 6, wherein the nucleic acid sequence of nanostructures is further modified chemically at the 5′-end and the nucleic acid segment sequence of the location identifiers complementary to a portion of the nucleic acid sequence of the modified moiety is modified chemically at the 5′-end to facilitate enzymatic or chemical ligation.

15. The method according to claim 1, wherein the moiety is an antibody, wherein the antibody is modified with at least two nucleic acid sequence strands.

16. (canceled)

17. A set of interacting nucleic acid structures for use in detecting and/or identifying a target comprising:
a nucleic acid sequence capable of being conjugated to a moiety directed to the target; and
a nucleic acid nanostructure comprising a segment sequence complementary to a portion of the nucleic acid sequence capable of being conjugated to a moiety directed to the target,
wherein the nucleic acid nanostructure comprises a tetrahedron.

18. (canceled)

19. The set of interacting nucleic acid structures according to claim 17, wherein the nucleic acid sequence is 50 nucleotides or less.

20. The set of interacting nucleic acid structures according to claim 17, further comprising at least two different location identifiers wherein each location identifier comprises a nucleic acid sequence comprising a segment sequence complementary to a second portion of the nucleic acid sequence of the modified moiety and a unique identifier, wherein the unique identifier comprises a nucleic acid sequence.

21. (canceled)

22. The set of interacting nucleic acid structures according to claim 20, wherein the at least two different location identifiers comprises at least three different location identifiers, a first location identifier wherein the unique identifier comprises a nucleic acid sequence a second location identifier wherein the unique identifier comprises a nucleic acid sequence conjugated to a nanoparticle having a diameter of 60 nm or less and a third location identifier wherein the unique identifier comprises a nucleic acid sequence conjugated to a nanoparticle having a diameter of 100 nm or more.

23. The set of interacting nucleic acid structures according to claim 20, further comprising at least two modified antibodies directed to at least two reference target proteins having a known cellular location.

24. The set of interacting nucleic acid structures according to claim 20, wherein the nucleic acid sequence capable of being conjugated to a moiety is conjugated to a moiety directed to the target, wherein the moiety comprises an antibody and the target is a protein target.

25-31. (canceled)

32. A method of diagnosing a disease comprising:
forming a modified antibody by conjugating a nucleic acid sequence to an antibody directed to a target protein associated with the disease;
forming a nucleic acid nanostructure comprising a segment sequence complementary to a portion of the nucleic acid sequence of the modified antibody;
forming at least two location identifiers wherein each location identifier comprises a nucleic acid sequence comprising a segment sequence complementary to a second portion of the nucleic acid sequence of the modified antibody and a unique identifier;
incubating a sample with the modified antibody to form a complex between the modified antibody and the target protein, and removing modified antibodies that do not form a complex with the target protein;
incubating the complex with the nucleic acid nanostructure, and at least one location identifier to form a super complex between the modified antibody, the target protein and the at least one of the location identifier;
ligating the nucleic acid of the super-complex between the segment sequence complementary to the portion of the nucleic acid nanostructure and the complementary segment sequence of the location identifier;
forming a nucleic acid barcode comprising the ligated sequence complementary to the portion of the nucleic acid sequence of the modified antibody and the sequence complementary to the second portion of the nucleic acid sequence of the modified antibody; and
detecting and analyzing the nucleic acid barcode to determine the amount and/or subcellular distribution of target proteins, whereby the amount and/or subcellular distribution of target protein indicates a disease.

33. The method according to claim 32, further comprising:
measuring at least two reference target proteins, each having a known cellular location using a modified antibody directed to each reference target protein by determining the relative location of the at least two reference target proteins, each having a different known cellular distribution, with a set of interacting nucleic acid structures comprising a modified antibody having a nucleic acid sequence conjugated thereto directed to each reference target protein; a nucleic acid nanostructure comprising a segment sequence complementary to a portion of the nucleic acid sequence conjugated to the modified antibody by detecting at least two reference barcodes formed comprising the complementary segment sequence of the nanostructure, the complementary segment sequence of the second portion of the nucleic acid sequence of the modified antibody and a unique identifier;
determining the amount of at least two target proteins with a set of interacting nucleic acid structures comprising a modified moiety having a nucleic acid sequence conjugated thereto directed to each target protein; a nucleic acid nanostructure comprising a segment sequence complementary to a portion of the nucleic acid sequence conjugated to the modified moiety by detecting; at least two barcodes comprising the complementary segment sequence of the nanostructure, the complementary segment sequence of the second portion of the nucleic acid sequence of the modified moiety and a unique identifier;
analyzing the at least two reference barcodes formed when the reference target protein is present and comparing the relative distribution of the at least two reference barcodes with the at least two barcodes formed when the target protein is present and determining the relative cellular location of the target protein.

34. The method according to claim 33, wherein the relative cellular location of the target protein is determined via a matrix conversion.

35. The method according to claim 32, wherein the disease is a disease subtype.

36. The method according to claim 35, wherein the disease subtype is a cancer subtype or an aggressive cancer subtype.

37. The method according to claim 35, wherein the disease subtype, the cancer subtype or the aggressive cancer subtype is breast cancer.

38. The method according to claim 37, wherein detection of more of the nucleic acid barcode comprising a unique identifier located in a nucleus compared to the nucleic acid barcode comprising a unique identifier located in other subcellular locations indicates the breast cancer is aggressive.

39. The method according to claim 35, wherein the target protein associated with cancer is selected from the group consisting of estrogen receptor (ER), progesterone receptor (PR), human epidermal growth factor receptor 2 (HER2) and a combination thereof.

40. The method according to claim 39, wherein the breast cancer is subtyped into luminal, non-luminal or triple negative based the expression of ER, PR and HER2, wherein detection of the nucleic acid barcode comprising the segment sequence complementary to the portion of the nucleic acid sequence of the modified antibody directed to the ER, and/or detection of the nucleic acid barcode comprising the segment sequence complementary to the portion of the nucleic acid sequence of the modified antibody directed to the PR, and/or detection of the nucleic acid barcode comprising the segment sequence complementary to the portion of the nucleic acid sequence of the modified antibody directed to the HER2 indicates the breast cancer is a luminal subtype; or wherein detection of the nucleic acid barcode comprising the segment sequence complementary to the portion of the nucleic acid sequence of the modified antibody directed to HER2 but not the segment sequence complementary to the portion of the nucleic acid sequence of the modified antibody directed to ER or PR indicates the breast cancer is a non-luminal subtype; or wherein the absence of the nucleic acid barcode comprising the segment sequence complementary to the portion of the nucleic acid sequence of the modified antibody directed to the ER, PR and HER2 indicates the breast cancer is a triple negative subtype.


SEQUENCE LISTING (95 sequences)

SEQ ID NO: 1 - DNA, 30 bp, Artificial Sequence
  Short DNA
acgggtatgatacttctatgatcgtacgat

SEQ ID NO: 2 - DNA, 79 bp, Artificial Sequence
  Long DNA
acgaacattcctaagtctgaaatttatcacccgccatatagacgtatcaccaggcagttgagttatcgtacgatcatag

SEQ ID NO: 3 - DNA, 80 bp, Artificial Sequence
  Tetrahedron 1
acgaacattcctaagtctgaaatttatcacccgccatagtagacgtatcaccaggcagttgagttatcgtacgatcatag

SEQ ID NO: 4 - DNA, 80 bp, Artificial Sequence
  Tetrahedron 2
attcagacttaggaatgttcgacatgcgagggtccaataccgacgattacagcttgctacacgtttacagtcgtattgca

SEQ ID NO: 5 - DNA, 80 bp, Artificial Sequence
  Tetrahedron 3
actactatggcgggtgataaaacgtgtagcaagctgtaatcgacgggaagagcatgcccatccttattctagacgttact

SEQ ID NO: 6 - DNA, 80 bp, Artificial Sequence
  Tetrahedron 4
acggtattggaccctcgcatgactcaactgcctggtgatacgaggatgggcatgctcttcccgtttaactatagctacaa

SEQ ID NO: 7 - DNA, 28 bp, Artificial Sequence
  Overhang DNA
aagtatcatacccgtctccacgaaaaaa

SEQ ID NO: 8 - DNA, 80 bp, Artificial Sequence; identical to SEQ ID NO: 3
  Tetrahedron 1 Click

SEQ ID NO: 9 - DNA, 28 bp, Artificial Sequence
  Overhang DNA Label Click
aagtatcatacccgtctccacgaaaaaa

SEQ ID NO: 10 - DNA, 20 bp, Artificial Sequence
  Forward primer
atcacccgccatagtagacg

SEQ ID NO: 11 - DNA, 22 bp, Artificial Sequence
  Reverse primer
cgtggagacgggtatgatactt

SEQ ID NO: 12 - DNA, 30 bp, Artificial Sequence; identical to SEQ ID NO: 1
  Ab-Tetrahedron 1

SEQ ID NO: 13 - DNA, 30 bp, Artificial Sequence
  Ab-Tetrahedron 2
acgggtatgatactttgcaatacgactgta

SEQ ID NO: 14 - DNA, 30 bp, Artificial Sequence
  Ab-Tetrahedron 3
acgggtatgatacttagtaacgtctagaat

SEQ ID NO: 15 - DNA, 30 bp, Artificial Sequence
  Ab-Tetrahedron 4
acgggtatgatacttttgtagctatagtta

SEQ ID NO: 16 - DNA, 38 bp, Artificial Sequence
  L1
aagtatcatacccgtctccacgacttagaatcaaaaaa

SEQ ID NO: 17 - DNA, 38 bp, Artificial Sequence
  L2
aagtatcatacccgtagttgctggacgattgtaaaaaa

SEQ ID NO: 18 - DNA, 38 bp, Artificial Sequence
  L3
aagtatcatacccgttcaccgtagctcaatggaaaaaa

SEQ ID NO: 19 - DNA, 118 bp, Artificial Sequence
  Tetrahedron 1-L1
acgaacattcctaagtctgaaatttatcacccgccatagtagacgtatcaccaggcagttgagttatcgtacgatcatagaagtatcatacccgtctccacgacttagaatcaaaaaa

SEQ ID NO: 20 - DNA, 20 bp, Artificial Sequence
  forward primer
atcacccgccatagtagacg

SEQ ID NO: 21 - DNA, 21 bp, Artificial Sequence
  reverse primer
gattctaagtcgtggagacgg

SEQ ID NO: 22 - DNA, 118 bp, Artificial Sequence
  Tetrahedron 1-L2
acgaacattcctaagtctgaaatttatcacccgccatagtagacgtatcaccaggcagttgagttatcgtacgatcatagaagtatcatacccgtagttgctggacgattgtaaaaaa

SEQ ID NO: 23 - DNA, 20 bp, Artificial Sequence
  forward primer
atcacccgccatagtagacg

SEQ ID NO: 24 - DNA, 20 bp, Artificial Sequence
  reverse primer
acaatcgtccagcaactacg

SEQ ID NO: 25 - DNA, 118 bp, Artificial Sequence
  Tetrahedron 1-L3
acgaacattcctaagtctgaaatttatcacccgccatagtagacgtatcaccaggcagttgagttatcgtacgatcatagaagtatcatacccgttcaccgtagctcaatggaaaaaa

SEQ ID NO: 26 - DNA, 20 bp, Artificial Sequence
  Forward primer
atcacccgccatagtagacg

SEQ ID NO: 27 - DNA, 20 bp, Artificial Sequence
  Reverse primer
ccattgagctacggtgaacg

SEQ ID NO: 28 - DNA, 118 bp, Artificial Sequence
  Tetrahedron 2-L1
attcagacttaggaatgttcgacatgcgagggtccaataccgacgattacagcttgctacacgtttacagtcgtattgcaaagtatcatacccgtctccacgacttagaatcaaaaaa

SEQ ID NO: 29 - DNA, 20 bp, Artificial Sequence
  Forward primer
gacatgcgagggtccaatac

SEQ ID NO: 30 - DNA, 21 bp, Artificial Sequence
  Reverse primer
gattctaagtcgtggagacgg

SEQ ID NO: 31 - DNA, 118 bp, Artificial Sequence
  Tetrahedron 2-L2
attcagacttaggaatgttcgacatgcgagggtccaataccgacgattacagcttgctacacgtttacagtcgtattgcaaagtatcatacccgtagttgctggacgattgtaaaaaa

SEQ ID NO: 32 - DNA, 20 bp, Artificial Sequence
  Forward primer
gacatgcgagggtccaatac

SEQ ID NO: 33 - DNA, 20 bp, Artificial Sequence
  Reverse primer
acaatcgtccagcaactacg

SEQ ID NO: 34 - DNA, 118 bp, Artificial Sequence
  Tetrahedron 2-L3
attcagacttaggaatgttcgacatgcgagggtccaataccgacgattacagcttgctacacgtttacagtcgtattgcaaagtatcatacccgttcaccgtagctcaatggaaaaaa

SEQ ID NO: 35 - DNA, 20 bp, Artificial Sequence
  Forward primer
gacatgcgagggtccaatac

SEQ ID NO: 36 - DNA, 20 bp, Artificial Sequence
  Reverse primer
ccattgagctacggtgaacg

SEQ ID NO: 37 - DNA, 118 bp, Artificial Sequence
  Tetrahedron 3-L1
actactatggcgggtgataaaacgtgtagcaagctgtaatcgacgggaagagcatgcccatccttattctagacgttactaagtatcatacccgtctccacgacttagaatcaaaaaa

SEQ ID NO: 38 - DNA, 20 bp, Artificial Sequence
  Forward primer
agctgtaatcgacgggaaga

SEQ ID NO: 39 - DNA, 21 bp, Artificial Sequence
  Reverse primer
gattctaagtcgtggagacgg

SEQ ID NO: 40 - DNA, 118 bp, Artificial Sequence
  Tetrahedron 3-L2
actactatggcgggtgataaaacgtgtagcaagctgtaatcgacgggaagagcatgcccatccttattctagacgttactaagtatcatacccgtagttgctggacgattgtaaaaaa

SEQ ID NO: 41 - DNA, 20 bp, Artificial Sequence
  Forward primer
agctgtaatcgacgggaaga

SEQ ID NO: 42 - DNA, 20 bp, Artificial Sequence
  Reverse primer
acaatcgtccagcaactacg

SEQ ID NO: 43 - DNA, 118 bp, Artificial Sequence
  Tetrahedron 3-L3
actactatggcgggtgataaaacgtgtagcaagctgtaatcgacgggaagagcatgcccatccttattctagacgttactaagtatcatacccgttcaccgtagctcaatggaaaaaa

SEQ ID NO: 44 - DNA, 20 bp, Artificial Sequence
  Forward primer
agctgtaatcgacgggaaga

SEQ ID NO: 45 - DNA, 20 bp, Artificial Sequence
  Reverse primer
ccattgagctacggtgaacg

SEQ ID NO: 46 - DNA, 118 bp, Artificial Sequence
  Tetrahedron 4-L1
acggtattggaccctcgcatgactcaactgcctggtgatacgaggatgggcatgctcttcccgtttaactatagctacaaaagtatcatacccgtctccacgacttagaatcaaaaaa

SEQ ID NO: 47 - DNA, 20 bp, Artificial Sequence
  Forward primer
tcaactgcctggtgatacga

SEQ ID NO: 48 - DNA, 21 bp, Artificial Sequence
  Reverse primer
gattctaagtcgtggagacgg

SEQ ID NO: 49 - DNA, 118 bp, Artificial Sequence
  Tetrahedron 4-L2
acggtattggaccctcgcatgactcaactgcctggtgatacgaggatgggcatgctcttcccgtttaactatagctacaaaagtatcatacccgtagttgctggacgattgtaaaaaa

SEQ ID NO: 50 - DNA, 20 bp, Artificial Sequence
  Forward primer
tcaactgcctggtgatacga

SEQ ID NO: 51 - DNA, 20 bp, Artificial Sequence
  Reverse primer
acaatcgtccagcaactacg

SEQ ID NO: 52 - DNA, 118 bp, Artificial Sequence
  Tetrahedron 4-L3
acggtattggaccctcgcatgactcaactgcctggtgatacgaggatgggcatgctcttcccgtttaactatagctacaaaagtatcatacccgttcaccgtagctcaatggaaaaaa

SEQ ID NO: 53 - DNA, 20 bp, Artificial Sequence
  Forward primer
tcaactgcctggtgatacga

SEQ ID NO: 54 - DNA, 20 bp, Artificial Sequence
  Reverse primer
ccattgagctacggtgaacg

SEQ ID NO: 55 - DNA, 59 bp, Artificial Sequence
  Tetrahedron 1 A
  misc_feature (34)..(39): n is a, c, g, or t
acactctttccctacacgacgctcttccgatctnnnnnncccgccatagtagacgtatc

SEQ ID NO: 56 - DNA, 60 bp, Artificial Sequence
  Tetrahedron 1 B
  misc_feature (34)..(39): n is a, c, g, or t
acactctttccctacacgacgctcttccgatctnnnnnncgccatagtagacgtatcacc

SEQ ID NO: 57 - DNA, 61 bp, Artificial Sequence
  Tetrahedron 1 C
  misc_feature (34)..(39): n is a, c, g, or t
acactctttccctacacgacgctcttccgatctnnnnnngccatagtagacgtatcaccag

SEQ ID NO: 58 - DNA, 60 bp, Artificial Sequence
  Tetrahedron 1 D
  misc_feature (34)..(39): n is a, c, g, or t
acactctttccctacacgacgctcttccgatctnnnnnntagtagacgtatcaccaggca

SEQ ID NO: 59 - DNA, 59 bp, Artificial Sequence
  Tetrahedron 1 E
  misc_feature (34)..(39): n is a, c, g, or t
acactctttccctacacgacgctcttccgatctnnnnnnagtagacgtatcaccaggca

SEQ ID NO: 60 - DNA, 59 bp, Artificial Sequence
  Tetrahedron 2 A
  misc_feature (34)..(39): n is a, c, g, or t
acactctttccctacacgacgctcttccgatctnnnnnngggtccaataccgacgatta

SEQ ID NO: 61 - DNA, 60 bp, Artificial Sequence
  Tetrahedron 2 B
  misc_feature (34)..(39): n is a, c, g, or t
acactctttccctacacgacgctcttccgatctnnnnnnggtccaataccgacgattaca

SEQ ID NO: 62 - DNA, 60 bp, Artificial Sequence
  Tetrahedron 2 C
  misc_feature (34)..(39): n is a, c, g, or t
acactctttccctacacgacgctcttccgatctnnnnnntccaataccgacgattacagc

SEQ ID NO: 63 - DNA, 61 bp, Artificial Sequence
  Tetrahedron 2 D
  misc_feature (34)..(39): n is a, c, g, or t
acactctttccctacacgacgctcttccgatctnnnnnncaataccgacgattacagcttg

SEQ ID NO: 64 - DNA, 59 bp, Artificial Sequence
  Tetrahedron 2 E
  misc_feature (34)..(39): n is a, c, g, or t
acactctttccctacacgacgctcttccgatctnnnnnntaccgacgattacagcttgc

SEQ ID NO: 65 - DNA, 59 bp, Artificial Sequence
  Tetrahedron 3 A
  misc_feature (34)..(39): n is a, c, g, or t
acactctttccctacacgacgctcttccgatctnnnnnncaagctgtaatcgacgggaa

SEQ ID NO: 66 - DNA, 58 bp, Artificial Sequence
  Tetrahedron 3 B
  misc_feature (34)..(39): n is a, c, g, or t
acactctttccctacacgacgctcttccgatctnnnnnnagctgtaatcgacgggaag

SEQ ID NO: 67 - DNA, 59 bp, Artificial Sequence
  Tetrahedron 3 C
  misc_feature (34)..(39): n is a, c, g, or t
acactctttccctacacgacgctcttccgatctnnnnnnctgtaatcgacgggaagagc

SEQ ID NO: 68 - DNA, 59 bp, Artificial Sequence
  Tetrahedron 3 D
  misc_feature (34)..(39): n is a, c, g, or t
acactctttccctacacgacgctcttccgatctnnnnnngtaatcgacgggaagagcat

SEQ ID NO: 69 - DNA, 58 bp, Artificial Sequence
  Tetrahedron 3 E
  misc_feature (34)..(39): n is a, c, g, or t
acactctttccctacacgacgctcttccgatctnnnnnnaatcgacgggaagagcatg

SEQ ID NO: 70 - DNA, 58 bp, Artificial Sequence
  Tetrahedron 4 A
  misc_feature (34)..(39): n is a, c, g, or t
acactctttccctacacgacgctcttccgatctnnnnnncctggtgatacgaggatgg

SEQ ID NO: 71 - DNA, 58 bp, Artificial Sequence
  Tetrahedron 4 B
  misc_feature (34)..(39): n is a, c, g, or t
acactctttccctacacgacgctcttccgatctnnnnnnctggtgatacgaggatggg

SEQ ID NO: 72 - DNA, 58 bp, Artificial Sequence
  Tetrahedron 4 C
  misc_feature (34)..(39): n is a, c, g, or t
acactctttccctacacgacgctcttccgatctnnnnnngtgatacgaggatgggcat

SEQ ID NO: 73 - DNA, 58 bp, Artificial Sequence
  Tetrahedron 4 D
  misc_feature (34)..(39): n is a, c, g, or t
acactctttccctacacgacgctcttccgatctnnnnnntgatacgaggatgggcatg

SEQ ID NO: 74 - DNA, 57 bp, Artificial Sequence
  Tetrahedron 4 E
  misc_feature (34)..(39): n is a, c, g, or t
acactctttccctacacgacgctcttccgatctnnnnnnatacgaggatgggcatgc

SEQ ID NO: 75 - DNA, 54 bp, Artificial Sequence
  Label
gtgactggagttcagacgtgtgctcttccgatctcgtggagacgggtatgatac

SEQ ID NO: 76 - DNA, 45 bp, Artificial Sequence
  Round 2 Read 1
aatgatacggcgaccaccgagatctacactctttccctacacgac

SEQ ID NO: 77 - DNA, 49 bp, Artificial Sequence
  Round 2 Read 2
  misc_feature (25)..(30): one of the below listed index SEQ
caagcagaagacggcatacgagatnnnnnngtgactggagttcagacgt

SEQ ID NO: 78 - DNA, 6 bp, Artificial Sequence
  Index 1
cgtgat

SEQ ID NO: 79 - DNA, 6 bp, Artificial Sequence
  Index 2
acatcg

SEQ ID NO: 80 - DNA, 6 bp, Artificial Sequence
  Index 3
gcctaa

SEQ ID NO: 81 - DNA, 6 bp, Artificial Sequence
  Index 4
tggtca

SEQ ID NO: 82 - DNA, 6 bp, Artificial Sequence
  Index 5
cactgt

SEQ ID NO: 83 - DNA, 6 bp, Artificial Sequence
  Index 6
attggc

SEQ ID NO: 84 - DNA, 6 bp, Artificial Sequence
  Index 7
gatctg

SEQ ID NO: 85 - DNA, 6 bp, Artificial Sequence
  Index 8
tcaagt

SEQ ID NO: 86 - DNA, 6 bp, Artificial Sequence
  Index 9
ctgatc

SEQ ID NO: 87 - DNA, 6 bp, Artificial Sequence
  Index 10
aagcta

SEQ ID NO: 88 - DNA, 6 bp, Artificial Sequence
  Index 11
gtagcc

SEQ ID NO: 89 - DNA, 6 bp, Artificial Sequence
  Index 12
tacaag

SEQ ID NO: 90 - DNA, 15 bp, Artificial Sequence
  complementary to first portion
aagtatcatacccgt

SEQ ID NO: 91 - DNA, 6 bp, Artificial Sequence
  complementary to second portion
atcata

SEQ ID NO: 92 - DNA, 15 bp, Artificial Sequence
  complementary to second portion
aagtatcatacccgt

SEQ ID NO: 93 - DNA, 17 bp, Artificial Sequence
  unique identifier
ctccacgacttagaatc

SEQ ID NO: 94 - DNA, 17 bp, Artificial Sequence
  unique identifier
agttgctggacgattgt

SEQ ID NO: 95 - DNA, 17 bp, Artificial Sequence
  unique identifier
tcaccgtagctcaatgg